(19) United States
(12) Patent Application Publication
(10) Pub. No.: US 2022/0040305 A1
(43) Pub. Date: Feb. 10, 2022

(54) FORMULATIONS FOR HIGHLY PURIFIED VIRAL PARTICLES

(71) Applicant: JANSSEN BIOTECH, INC., Horsham, PA (US)

(72) Inventors: Brian E. TOMKOWICZ, North Wales, PA (US); Matthew P. ERCOLINO, Stowe, PA (US); Stephen T. SPAGNOL, Conshohocken, PA (US); Sakya Sing MOHAPATRA, Conshohocken, PA (US); Jeffrey SMITH, Perkasie, PA (US)

(73) Assignee: JANSSEN BIOTECH, INC., Horsham, PA (US)

(21) Appl. No.: 17/396,090

(22) Filed: Aug. 6, 2021

Related U.S. Application Data
(60) Provisional application No. 63/063,128, filed on Aug. 7, 2020.
(60) Provisional application No. 63/063,108, filed on Aug. 7, 2020.

(51) Int. Cl. A61K 47/02; C12N 7/00; A61K 47/18; A61K 47/22; A61K 47/26; A61K 47/34

(52) U.S. Cl. CPC A61K 47/02; C12N 7/00; A61K 47/18; A61K 48/00; A61K 47/26; A61K 47/34; C12N 2750/14123; A61K 47/22

(57) ABSTRACT

Formulations for highly purified viral particles (e.g., adeno-associated virus (AAV) particles) are provided herein. The formulations include purified AAV particles that are substantially free of impurities (e.g., product-related impurities and process-related impurities), and one or more of a buffering agent, a cryoprotectant, a non-ionic surfactant, and optionally a pharmaceutically acceptable salt. In certain aspects, the formulation maintains or enhances stability and/or reduces or prevents aggregation of the purified AAV particles.

### CROSS-REFERENCE TO RELATED APPLICATIONS

This application claims the benefit of U.S. Ser. No. 63/063,108 filed Aug. 7, 2020, and U.S. Ser. No. 63/063,128 filed Aug. 7, 2020, the disclosure of each of which is incorporated by reference herein in its entirety.

### 1. FIELD

Provided herein are, among other things, formulations for highly purified viral particles (e.g., adeno-associated virus (AAV) particles). The formulations can include purified AAV particles that are substantially free of impurities (e.g., product-related impurities and process-related impurities), and one or more of a buffering agent, a cryoprotectant, a non-ionic surfactant, and optionally a pharmaceutically acceptable salt. In certain aspects, the formulations provided herein maintain or enhance stability and/or reduce or prevent aggregation of the purified AAV particles.

### 2. BACKGROUND

Adeno-associated virus (AAV) is a non-enveloped virus that can be engineered to deliver nucleic acids to target cells, and has emerged as a useful vehicle in gene therapy and gene delivery applications. Recombinant AAV (rAAV), which lacks viral DNA, is essentially a protein-based nanoparticle engineered to traverse the cell membrane, where it can ultimately traffic and deliver its nucleic acid cargo into the nucleus of a cell. The properties conferred by this virus: sustained gene expression, naturally occurring in the human population with wide tissue tropism, non-integrating, non-pathogenic, low immunogenicity, infectivity of post-mitotic cells and relative ease of production, when compared to other viral systems, have ushered in the rapid expansion for human use. Gene delivery vectors based on AAV, including rAAV, have emerged as safe and effective for numerous clinical gene therapy applications.

Production and purification of AAV particles remains a major challenge. Further, once purified, the development of a formulation capable of maintaining viral infectivity and physical stability becomes paramount to ensure high-quality products with consistent bioperformance are released to the patients. One particular concern for viral vectors is their propensity to aggregate (see Wright et al., Molecular Therapy, 2005). Thus, provided herein is a novel AAV formulation that addresses the unmet need of a formulation containing highly pure AAV particles with improved stability and minimal aggregation.

### 3. SUMMARY

In one aspect, provided herein is a pharmaceutical composition comprising a purified AAV particle, a buffering agent, a cryoprotectant, and a non-ionic surfactant, wherein (a) the purified AAV particle is substantially free of an impurity, (b) the buffering agent concentration is about 0 mM to about 50 mM, (c) the cryoprotectant is about 1% to about 10% (w/v), and (d) the non-ionic surfactant is about 0.0001% (w/v) to about 0.1% (w/v). In one embodiment, the pharmaceutical composition includes a pharmaceutically acceptable salt, wherein the pharmaceutically acceptable salt concentration is about 1 mM to about 200 mM. In one embodiment, the pharmaceutically acceptable salt is about 10 mM to about 150 mM. In one embodiment, the pharmaceutically acceptable salt concentration is about 10 mM. In one embodiment, the pharmaceutically acceptable salt concentration is about 100 mM. In one embodiment, the pharmaceutically acceptable salt concentration is about 150 mM.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 1 mM to about 49 mM, (c) the buffering agent concentration is about 0 mM to about 50 mM, (d) the cryoprotectant is about 1% to about 10% (w/v), and (e) the non-ionic surfactant is about 0.0001% (w/v) to about 0.1% (w/v). In one embodiment, the pharmaceutically acceptable salt concentration is about 5 mM to about 45 mM. In one embodiment, the pharmaceutically acceptable salt concentration is about 7.5 mM to about 40 mM. In one embodiment, the pharmaceutically acceptable salt concentration is about 10 mM to about 30 mM. In one embodiment, the pharmaceutically acceptable salt concentration is about 10 mM.

In one embodiment, pharmaceutically acceptable salt is selected from the group consisting of a sodium salt, a magnesium salt, a calcium salt, a potassium salt, a phosphate salt, and a sulfate salt. In one embodiment, the sodium salt comprises sodium chloride.

In one embodiment, the buffering agent comprises Tris hydrochloride (HCl). In one embodiment, the buffering agent comprises L-Histidine HCl. In one embodiment, the buffering agent concentration comprises about 20 mM.

In one embodiment, the cryoprotectant is about 3% (w/v) to about 8% (w/v). In one embodiment, the cryoprotectant is about 4% (w/v) to about 6% (w/v). In one embodiment, the cryoprotectant is about 5% (w/v). In one embodiment, the cryoprotectant comprises a sugar. In one embodiment, the sugar comprises sucrose, trehalose, or a combination thereof. In one embodiment, the sugar comprises trehalose.

In one embodiment, the non-ionic surfactant is about 0.0005% (w/v) to about 0.005% (w/v). In one embodiment, the non-ionic surfactant is about 0.00075% (w/v) to about 0.0025% (w/v). In one embodiment, the non-ionic surfactant is about 0.001% (w/v). In one embodiment, the non-ionic surfactant is selected from the group consisting of a copolymer, a polyoxyethylene sorbitan ester, a phospholipid, a Brij surfactant, and a sorbitan ester, or a combination thereof. In one embodiment, the polyoxyethylene sorbitan ester is selected from the group consisting of (PS-20), and polysorbate 80 (PS-80), or a combination thereof. In one embodiment, the copolymer comprises a poloxamer. In one embodiment, the poloxamer is selected from the group consisting of poloxamer 188 (P188), poloxamer 237 (P237), poloxamer 338 (P338), and poloxamer 407 (P407), or a combination thereof. In one embodiment, the poloxamer comprises poloxamer 188 (P188).

In one aspect, provided herein is a pharmaceutical composition comprising a purified AAV particle, a buffering agent, a cryoprotectant, and a non-ionic surfactant, wherein (a) the purified AAV particle is substantially free of an impurity, (b) the buffering agent concentration is about 20 mM, (c) the cryoprotectant is about 5% (w/v) trehalose, and (d) the non-ionic surfactant is about 0.001% (w/v) poloxamer 188.

In one aspect, provided herein is pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 10 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 25 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 50 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 100 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 125 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 150 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 200 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one embodiment, the buffering agent comprises Tris HCl. In one embodiment, the buffering agent comprises L-Histidine HCl.

In one embodiment, the pharmaceutical composition pH is about 4.0 to about 9.0. In one embodiment, the pharmaceutical composition pH is about 7.0 to about 8.0. In one embodiment, the pharmaceutical composition pH is about 7.3 to about 7.7. In one embodiment, the pharmaceutical composition pH is about 7.5.

In one embodiment, the AAV is selected from the group consisting of AAV1, AAV2, AAV3, AAV4, AAV5, AAV6, AAV7, AAV8, AAV9, and AAV10. In one embodiment, the AAV comprises a rAAV.

In one embodiment, the purified AAV particle titer is about 1×10¹⁰ viral genomes per milliliter (vg/mL) or greater. In one embodiment, the purified AAV particle titer is about 1×10¹¹ viral genomes per milliliter (vg/mL) or greater. In one embodiment, the purified AAV particle titer is about 1×10¹² viral genomes per milliliter (vg/mL) or greater. In one embodiment, the purified AAV particle titer is about 1×10¹³ viral genomes per milliliter (vg/mL) or greater. In one embodiment, the purified AAV particle titer is about 1×10¹⁴ viral genomes per milliliter (vg/mL) or greater. In one embodiment, the purified AAV particle titer is about 1×10¹⁵ viral genomes per milliliter (vg/mL) or greater.

In one embodiment, the impurity comprises a process-related impurity, a product-related impurity, or a combination thereof.

In one embodiment, the process-related impurity is selected from the group consisting of a residual host-cell component, a residual viral production component, a residual cell culture component, a residual purification component, or a combination thereof. In one embodiment, the residual host-cell component comprises a host-cell protein, a host-cell DNA, a host-cell RNA, or a combination thereof. In one embodiment, the host-cell DNA comprises an extra-viral, chromatin-associated DNA. In one embodiment, the residual viral production component comprises a plasmid DNA, a helper virus, or a combination thereof. In one embodiment, the residual cell culture component comprises an antibiotic, a supplement, an inducer, a growth factor, or a combination thereof. In one embodiment, the residual purification component comprises a buffer, an inorganic salt, an enzyme, a detergent, a medium, or a combination thereof.

In one embodiment, the product-related impurity comprises an empty capsid, an aggregated AAV particle, a degraded AAV particle, or a combination thereof.

In one embodiment, the purified AAV particle comprises a full or a partially-full capsid, and the product-related impurity comprises an empty capsid. In one embodiment, the purified AAV particle comprises a full capsid, and the product-related impurity comprises an empty capsid. In one embodiment, the purified AAV particle consists essentially of a full capsid, and the product-related impurity comprises an empty capsid.

In one embodiment, the product-related impurity comprises an aggregated AAV particle, a degraded AAV particle, or a combination thereof. In one embodiment, the purified AAV particle comprises an empty capsid. In one embodiment, the purified AAV particle consists essentially of an empty capsid. In one embodiment, the product-related impurity comprises an aggregated AAV particle or a combination thereof.

In one embodiment, the pharmaceutical composition is in a liquid state. In one embodiment, the pharmaceutical composition is in a solid or a semi-solid state.

In one embodiment, the pharmaceutical composition maintains or enhances the stability of the purified AAV particle.

In one embodiment, the pharmaceutical composition reduces or prevents aggregation of the purified AAV particle.

In one embodiment, the pharmaceutical composition (a) maintains or enhances the stability of the purified AAV particle; and (b) reduces or prevents aggregation of the purified AAV particle.

In one embodiment, the stability of the purified AAV particle is maintained or enhanced after one or more freeze/thaw cycles. In one embodiment, the stability of the purified AAV particle is maintained or enhanced after three or more freeze/thaw cycles.

In one embodiment, the aggregation of the AAV particle is less than 5% after one or more freeze/thaw cycles. In one embodiment, the aggregation of the AAV particle is less than 2% after one or more freeze/thaw cycles. In one embodiment, the aggregation of the AAV particle is less than 1% after one or more freeze/thaw cycles. In one embodiment, the aggregation of the AAV particle is less than 5% after three or more freeze/thaw cycles. In one embodiment, the aggregation of the AAV particle is less than 2% after three or more freeze/thaw cycles. In one embodiment, the aggregation of the AAV particle is less than 1% after three or more freeze/thaw cycles.

In one embodiment, the stability and/or aggregation of the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

In one embodiment, the purified AAV particle is obtained by a method comprising: (a) contacting a supernatant comprising AAV particles with a composition comprising a nuclease; and (b) purifying the particles. In one embodiment, the nuclease comprises Benzonase, or Benzonase® and a chromatin-DNA nuclease. In one embodiment, the chromatin-DNA nuclease comprises a MNase.

In one aspect, provided herein is a method for making a pharmaceutical composition comprising a purified AAV particle, the method comprising: (a) contacting a supernatant comprising an AAV particle with a composition comprising a nuclease; (b) purifying the AAV particle, such that the AAV particle is substantially free of an impurity; (c) combining the purified AAV particle with a buffering agent, a cryoprotectant, and a non-ionic surfactant, wherein (i) the buffering agent concentration is about 0 mM to about 50 mM, (ii) the cryoprotectant is about 1% to about 10% (w/v), and (iii) the non-ionic surfactant is about 0.0001% (w/v) to about 0.1% (w/v). In one embodiment, step (c) further comprises a pharmaceutically acceptable salt, wherein the pharmaceutically acceptable salt concentration is about 1 mM to about 200 mM.

In one embodiment, the pharmaceutically acceptable salt is about 10 mM to about 150 mM. In one embodiment, the pharmaceutically acceptable salt is about 1 mM to about 49 mM. In one embodiment, the pharmaceutically acceptable salt is about 5 mM to about 45 mM. In one embodiment, the pharmaceutically acceptable salt is about 7.5 mM to about 40 mM. In one embodiment, the pharmaceutically acceptable salt is about 10 mM to about 30 mM. In one embodiment, the pharmaceutically acceptable salt concentration is about 10 mM. In one embodiment, the pharmaceutically acceptable salt concentration is about 100 mM. In one embodiment, the pharmaceutically acceptable salt concentration is about 150 mM.

In one embodiment, the pharmaceutically acceptable salt is selected from the group consisting of a sodium salt, a magnesium salt, a calcium salt, a potassium salt, a phosphate salt, and a sulfate salt. In one embodiment, the sodium salt comprises sodium chloride.

In one embodiment, the buffering agent comprises Tris HCl. In one embodiment, the buffering agent comprises L-Histidine HCl. In one embodiment, the buffering agent concentration comprises about 20 mM.

In one embodiment, the cryoprotectant is about 3% (w/v) to about 8% (w/v). In one embodiment, the cryoprotectant is about 4% (w/v) to about 6% (w/v). In one embodiment, the cryoprotectant is about 5% (w/v). In one embodiment, the cryoprotectant comprises a sugar. In one embodiment, the sugar comprises sucrose, trehalose, or a combination thereof. In one embodiment, the sugar comprises trehalose.

In one embodiment, the non-ionic surfactant is about 0.0005% (w/v) to about 0.005% (w/v). In one embodiment, the non-ionic surfactant is about 0.00075% (w/v) to about 0.0025% (w/v). In one embodiment, the non-ionic surfactant is about 0.001% (w/v). In one embodiment, the non-ionic surfactant is selected from the group consisting of a copolymer, a polyoxyethylene sorbitan ester, a phospholipid, a Brij surfactant, and a sorbitan ester, or a combination thereof. In one embodiment, the polyoxyethylene sorbitan ester is selected from the group consisting of (PS-20), and polysorbate 80 (PS-80), or a combination thereof. In one embodiment, the copolymer comprises a poloxamer. In one embodiment, the poloxamer is selected from the group consisting of poloxamer 188 (P188), poloxamer 237 (P237), poloxamer 338 (P338), and poloxamer 407 (P407), or a combination thereof. In one embodiment, the poloxamer comprises poloxamer 188 (P188).

In one embodiment, the pharmaceutical composition pH is about 4.0 to about 9.0. In one embodiment, the pharmaceutical composition pH is about 7.0 to about 8.0. In one embodiment, the pharmaceutical composition pH is about 7.3 to about 7.7. In one embodiment, the pharmaceutical composition pH is about 7.5.

In one embodiment, the AAV is selected from the group consisting of AAV1, AAV2, AAV3, AAV4, AAV5, AAV6, AAV7, AAV8, AAV9, and AAV10. In one embodiment, the AAV comprises a rAAV.

In one embodiment, the nuclease comprises Benzonase, or Benzonase® and a chromatin-DNA nuclease. In one embodiment, the chromatin-DNA nuclease comprises a MNase.

In one embodiment, the purified AAV particle titer is about 1×10¹⁰ viral genomes per milliliter (vg/mL) or greater. In one embodiment, the purified AAV particle titer is about 1×10¹¹ viral genomes per milliliter (vg/mL) or greater. In one embodiment, the purified AAV particle titer is about 1×10¹² viral genomes per milliliter (vg/mL) or greater. In one embodiment, the purified AAV particle titer is about 1×10¹³ viral genomes per milliliter (vg/mL) or greater. In one embodiment, the purified AAV particle titer is about 1×10¹⁴ viral genomes per milliliter (vg/mL) or greater. In one embodiment, the purified AAV particle titer is about 1×10¹⁵ viral genomes per milliliter (vg/mL) or greater.

In one embodiment, the impurity comprises a process-related impurity, a product-related impurity, or a combination thereof.

In one embodiment, the process-related impurity is selected from the group consisting of a residual host-cell component, a residual viral production component, a residual cell culture component, a residual purification component, or a combination thereof. In one embodiment, the residual host-cell component comprises a host-cell protein, a host-cell DNA, a host-cell RNA, or a combination thereof. In one embodiment, the host-cell DNA comprises an extra-viral, chromatin-associated DNA. In one embodiment, the residual viral production component comprises a plasmid DNA, a helper virus, or a combination thereof. In one embodiment, the residual cell culture component comprises an antibiotic, a supplement, an inducer, a growth factor, or a combination thereof. In one embodiment, the residual purification component comprises a buffer, an inorganic salt, an enzyme, a detergent, a medium, or a combination thereof.

In one embodiment, the product-related impurity comprises an empty capsid, an aggregated AAV particle, a degraded AAV particle, or a combination thereof. In one embodiment, the purified AAV particle comprises a full or a partially-full capsid, and the product-related impurity comprises an empty capsid. In one embodiment, the purified AAV particle comprises a full capsid, and the product-related impurity comprises an empty capsid. In one embodiment, the purified AAV particle consists essentially of a full capsid, and the product-related impurity comprises an empty capsid.

In one embodiment, the product-related impurity comprises an aggregated AAV particle, a degraded AAV particle, or a combination thereof. In one embodiment, the purified AAV particle comprises an empty capsid. In one embodiment, the purified AAV particle consists essentially of an empty capsid. In one embodiment, the product-related impurity comprises an aggregated AAV particle.

In one aspect, provided herein is a pharmaceutical composition comprising a means for maintaining or enhancing the stability of a purified AAV particle. In one embodiment, the stability of the purified AAV particle is maintained or enhanced after one or more freeze/thaw cycles. In one embodiment, the stability of the purified AAV particle is maintained or enhanced after three or more freeze/thaw cycles. In one embodiment, the stability of the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

In one aspect, provided herein is a pharmaceutical composition comprising a means for decreasing or preventing aggregation of a purified AAV particle. In one embodiment, the aggregation of the AAV particle is less than 5% after one or more freeze/thaw cycles. In one embodiment, the aggregation of the AAV particle is less than 2% after one or more freeze/thaw cycles. In one embodiment, the aggregation of the AAV particle is less than 1% after one or more freeze/thaw cycles. In one embodiment, the aggregation of the AAV particle is less than 5% after three or more freeze/thaw cycles. In one embodiment, the aggregation of the AAV particle is less than 2% after three or more freeze/thaw cycles. In one embodiment, the aggregation of the AAV particle is less than 1% after three or more freeze/thaw cycles. In one embodiment, the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

In one aspect, provided herein is a pharmaceutical composition comprising a means for (a) for maintaining or enhancing the stability of a purified AAV particle, and (b) decreasing or preventing aggregation of a purified AAV particle. In one embodiment, the stability of the purified AAV particle is maintained or enhanced after one or more freeze/thaw cycles. In one embodiment, the stability of the purified AAV particle is maintained or enhanced after three or more freeze/thaw cycles. In one embodiment, the aggregation of the AAV particle is less than 5% after one or more freeze/thaw cycles. In one embodiment, the aggregation of the AAV particle is less than 2% after one or more freeze/thaw cycles. In one embodiment, the aggregation of the AAV particle is less than 1% after one or more freeze/thaw cycles. In one embodiment, the aggregation of the AAV particle is less than 5% after three or more freeze/thaw cycles. In one embodiment, the aggregation of the AAV particle is less than 2% after three or more freeze/thaw cycles. In one embodiment, the aggregation of the AAV particle is less than 1% after three or more freeze/thaw cycles. In one embodiment, the stability and aggregation of the AAV particle are measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

In one aspect, provided herein is a system comprising a means for making and obtaining a pharmaceutical composition comprising a purified AAV particle, a buffering agent, a cryoprotectant, and a non-ionic surfactant, wherein (a) the purified AAV particle is substantially free of an impurity, (b) the buffering agent concentration is about 0 mM to about 50 mM, (c) the cryoprotectant is about 1% to about 10% (w/v), and (d) the non-ionic surfactant is about 0.0001% (w/v) to about 0.1% (w/v). In one embodiment, the pharmaceutical composition further comprises a pharmaceutically acceptable salt, wherein the pharmaceutically acceptable salt concentration is about 1 mM to about 200 mM. In one embodiment, the pharmaceutically acceptable salt is about 10 mM to about 150 mM. In one embodiment, the pharmaceutically acceptable salt concentration is about 10 mM. In one embodiment, the pharmaceutically acceptable salt concentration is about 100 mM. In one embodiment, the pharmaceutically acceptable salt concentration is about 150 mM.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the buffering agent concentration is about 0 mM to about 50 mM, (c) the cryoprotectant is about 1% to about 10% (w/v), and (d) the non-ionic surfactant is about 0.0001% (w/v) to about 0.1% (w/v). In one embodiment, the pharmaceutical composition further includes a pharmaceutically acceptable salt, wherein the pharmaceutically acceptable salt concentration is about 1 mM to about 200 mM. In one embodiment, the pharmaceutically acceptable salt is about 10 mM to about 150 mM. In one embodiment, the pharmaceutically acceptable salt concentration is about 10 mM. In one embodiment, the pharmaceutically acceptable salt concentration is about 100 mM. In one embodiment, the pharmaceutically acceptable salt concentration is about 150 mM.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 1 mM to about 49 mM, (c) the buffering agent concentration is about 0 mM to about 50 mM, (d) the cryoprotectant is about 1% to about 10% (w/v), and (e) the non-ionic surfactant is about 0.0001% (w/v) to about 0.1% (w/v). In one embodiment, the pharmaceutically acceptable salt concentration is about 5 mM to about 45 mM. In one embodiment, the pharmaceutically acceptable salt concentration is about 7.5 mM to about 40 mM. In one embodiment, the pharmaceutically acceptable salt concentration is about 10 mM to about 30 mM. In one embodiment, the pharmaceutically acceptable salt concentration is about 10 mM.

In one embodiment, pharmaceutically acceptable salt is selected from the group consisting of a sodium salt, a magnesium salt, a calcium salt, a potassium salt, a phosphate salt, and a sulfate salt. In one embodiment, the sodium salt comprises sodium chloride.

In one embodiment, the buffering agent comprises Tris HCl. In one embodiment, the buffering agent comprises L-Histidine HCl. In one embodiment, the buffering agent concentration comprises about 20 mM.

In one embodiment, the cryoprotectant is about 3% (w/v) to about 8% (w/v). In one embodiment, the cryoprotectant is about 4% (w/v) to about 6% (w/v). In one embodiment, the cryoprotectant is about 5% (w/v). In one embodiment, the cryoprotectant comprises a sugar. In one embodiment, the sugar comprises sucrose, trehalose, or a combination thereof. In one embodiment, the sugar comprises trehalose.

In one embodiment, the non-ionic surfactant is about 0.0005% (w/v) to about 0.005% (w/v). In one embodiment, the non-ionic surfactant is about 0.00075% (w/v) to about 0.0025% (w/v). In one embodiment, the non-ionic surfactant is about 0.001% (w/v). In one embodiment, the non-ionic surfactant is selected from the group consisting of a copolymer, a polyoxyethylene sorbitan ester, a phospholipid, a Brij surfactant, and a sorbitan ester, or a combination thereof. In one embodiment, the polyoxyethylene sorbitan ester is selected from the group consisting of (PS-20), and polysorbate 80 (PS-80), or a combination thereof. In one embodiment, the copolymer comprises a poloxamer. In one embodiment, the poloxamer is selected from the group consisting of poloxamer 188 (P188), poloxamer 237 (P237), poloxamer 338 (P338), and poloxamer 407 (P407), or a combination thereof. In one embodiment, the poloxamer comprises poloxamer 188 (P188).

In one aspect, provided herein is a system comprising a means for making and obtaining a pharmaceutical composition comprising a purified AAV particle, a buffering agent, a cryoprotectant, and a non-ionic surfactant, wherein (a) the purified AAV particle is substantially free of an impurity, (b) the buffering agent concentration is about 20 mM, (c) the cryoprotectant is about 5% (w/v) trehalose, and (d) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 10 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 25 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 50 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 100 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 125 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 150 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one aspect, provided herein is a system comprising a means for making and obtaining a pharmaceutical composition comprising a purified AAV particle, a pharmaceutically acceptable salt, a buffering agent, a cryoprotectant, and a non-ionic surfactant, wherein (a) the purified AAV particle is substantially free of an impurity, (b) the pharmaceutically acceptable salt concentration is about 200 mM sodium chloride, (c) the buffering agent concentration is about 20 mM, (d) the cryoprotectant is about 5% (w/v) trehalose, and (e) the non-ionic surfactant is about 0.001% (w/v) poloxamer 188.

In one embodiment, the buffering agent comprises Tris HCl. In one embodiment, the buffering agent comprises L-Histidine HCl.

In one embodiment, the pharmaceutical composition pH is about 4.0 to about 9.0. In one embodiment, the pharmaceutical composition pH is about 7.0 to about 8.0. In one embodiment, the pharmaceutical composition pH is about 7.3 to about 7.7. In one embodiment, the pharmaceutical composition pH is about 7.5.

In one embodiment, the AAV is selected from the group consisting of AAV1, AAV2, AAV3, AAV4, AAV5, AAV6, AAV7, AAV8, AAV9, and AAV10. In one embodiment, the AAV comprises a rAAV.

In one embodiment, the purified AAV particle titer is about 1×10¹⁰ viral genomes per milliliter (vg/mL) or greater. In one embodiment, the purified AAV particle titer is about 1×10¹¹ viral genomes per milliliter (vg/mL) or greater. In one embodiment, the purified AAV particle titer is about 1×10¹² viral genomes per milliliter (vg/mL) or greater. In one embodiment, the purified AAV particle titer is about 1×10¹³ viral genomes per milliliter (vg/mL) or greater. In one embodiment, the purified AAV particle titer is about 1×10¹⁴ viral genomes per milliliter (vg/mL) or greater. In one embodiment, the purified AAV particle titer is about 1×10¹⁵ viral genomes per milliliter (vg/mL) or greater.

In one embodiment, the impurity comprises a process-related impurity, a product-related impurity, or a combination thereof.

In one embodiment, the process-related impurity is selected from the group consisting of a residual host-cell component, a residual viral production component, a residual cell culture component, a residual purification component, or a combination thereof. In one embodiment, the residual host-cell component comprises a host-cell protein, a host-cell DNA, a host-cell RNA, or a combination thereof. In one embodiment, the host-cell DNA comprises an extra-viral, chromatin-associated DNA. In one embodiment, the residual viral production component comprises a plasmid DNA, a helper virus, or a combination thereof. In one embodiment, the residual cell culture component comprises an antibiotic, a supplement, an inducer, a growth factor, or a combination thereof. In one embodiment, the residual purification component comprises a buffer, an inorganic salt, an enzyme, a detergent, a medium, or a combination thereof.

In one embodiment, the product-related impurity comprises an empty capsid, an aggregated AAV particle, a degraded AAV particle, or a combination thereof.

In one embodiment, the purified AAV particle comprises a full or a partially-full capsid, and the product-related impurity comprises an empty capsid. In one embodiment, the purified AAV particle comprises a full capsid, and the product-related impurity comprises an empty capsid. In one embodiment, the purified AAV particle consists essentially of a full capsid, and the product-related impurity comprises an empty capsid.

In one embodiment, the product-related impurity comprises an aggregated AAV particle, a degraded AAV particle, or a combination thereof. In one embodiment, the purified AAV particle comprises an empty capsid. In one embodiment, the purified AAV particle consists essentially of an empty capsid. In one embodiment, the product-related impurity comprises an aggregated AAV particle or a combination thereof.

In one embodiment, the pharmaceutical composition is in a liquid state. In one embodiment, the pharmaceutical composition is in a solid or a semi-solid state.

In one embodiment, the pharmaceutical composition maintains or enhances the stability of the purified AAV particle.

In one embodiment, the pharmaceutical composition reduces or prevents aggregation of the purified AAV particle.

In one embodiment, the pharmaceutical composition (a) maintains or enhances the stability of the purified AAV particle; and (b) reduces or prevents aggregation of the purified AAV particle.

In one embodiment, the stability of the purified AAV particle is maintained or enhanced after one or more freeze/thaw cycles. In one embodiment, the stability of the purified AAV particle is maintained or enhanced after three or more freeze/thaw cycles.

In one embodiment, the aggregation of the AAV particle is less than 5% after one or more freeze/thaw cycles. In one embodiment, the aggregation of the AAV particle is less than 2% after one or more freeze/thaw cycles. In one embodiment, the aggregation of the AAV particle is less than 1% after one or more freeze/thaw cycles. In one embodiment, the aggregation of the AAV particle is less than 5% after three or more freeze/thaw cycles. In one embodiment, the aggregation of the AAV particle is less than 2% after three or more freeze/thaw cycles. In one embodiment, the aggregation of the AAV particle is less than 1% after three or more freeze/thaw cycles.

In one embodiment, the stability and/or aggregation of the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

In one embodiment, the purified AAV particle is obtained by a method comprising: (a) contacting a supernatant comprising AAV particles with a composition comprising a nuclease; and (b) purifying the particles. In one embodiment, the nuclease comprises Benzonase, or Benzonase® and a chromatin-DNA nuclease. In one embodiment, the chromatin-DNA nuclease comprises a MNase.

### 4. BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1A-FIG. 1D illustrate AAV particle size data (pre-freeze) measured by dynamic light scattering (DLS) for four representative formulations. FIG. 1A depicts purified AAV particles in exemplary Formulation #1 (20 mM Tris-HCl pH 7.5, 10 mM NaCl, 5% Trehalose, and 0.001% Poloxamer 188). FIG. 1B depicts purified AAV particles in exemplary Formulation #2 (20 mM Tris-HCl pH 7.5, 100 mM NaCl, 5% Trehalose, and 0.001% Poloxamer 188). FIG. 1C depicts purified AAV particles in exemplary Formulation #3 (20 mM Tris-HCl pH 7.5, 150 mM NaCl, 5% Trehalose, and 0.001% Poloxamer 188). FIG. 1D depicts purified AAV particles in exemplary Formulation #4 (20 mM Tris-HCl pH 7.5, 200 mM NaCl, 5% Trehalose, and 0.001% Poloxamer 188).

FIG. 2A-FIG. 2D illustrate AAV particle size data after a single freeze/thaw cycle, measured by dynamic light scattering (DLS) for four representative formulations. FIG. 2A depicts purified AAV particles in exemplary Formulation #1 (20 mM Tris-HCl pH 7.5, 10 mM NaCl, 5% Trehalose, and 0.001% Poloxamer 188). FIG. 2B depicts purified AAV particles in exemplary Formulation #2 (20 mM Tris-HCl pH 7.5, 100 mM NaCl, 5% Trehalose, and 0.001% Poloxamer 188). FIG. 2C depicts purified AAV particles in exemplary Formulation #3 (20 mM Tris-HCl pH 7.5, 150 mM NaCl, 5% Trehalose, and 0.001% Poloxamer 188). FIG. 2D depicts purified AAV particles in exemplary Formulation #4 (20 mM Tris-HCl pH 7.5, 200 mM NaCl, 5% Trehalose, and 0.001% Poloxamer 188).

FIG. 3A-FIG. 3D illustrate AAV particle size data after three freeze/thaw cycles, measured by dynamic light scattering (DLS) for four representative formulations. FIG. 3A depicts purified AAV particles in exemplary Formulation #1 (20 mM Tris-HCl pH 7.5, 10 mM NaCl, 5% Trehalose, and 0.001% Poloxamer 188). FIG. 3B depicts purified AAV particles in exemplary Formulation #2 (20 mM Tris-HCl pH 7.5, 100 mM NaCl, 5% Trehalose, and 0.001% Poloxamer 188). FIG. 3C depicts purified AAV particles in exemplary Formulation #3 (20 mM Tris-HCl pH 7.5, 150 mM NaCl, 5% Trehalose, and 0.001% Poloxamer 188). FIG. 3D depicts purified AAV particles in exemplary Formulation #4 (20 mM Tris-HCl pH 7.5, 200 mM NaCl, 5% Trehalose, and 0.001% Poloxamer 188).

FIG. 4A and FIG. 4B illustrates an exemplary AAV purification method involving AAV particle purification with 60 U/mL of MNase treatment (FIG. 4B) or without AAV particle purification (FIG. 4A). The large 260 nm (RNA/DNA) absorbance contribution to the post-product peak is greatly reduced, as is the 280 nm (protein) absorbance peak, after MNase treatment.

FIG. 5A and FIG. 5B illustrate overlays of the chromatogram from rAAV8 particles containing samples treated with or without MNase. Addition of MNase caused a significant reduction in post-product peak heights for DNA/RNA (260 nm) (FIG. 5A) and protein (280 nm) (FIG. 5B), which indicated that post-product peaks are AAV particles containing extra-virally associated chromatin and that MNase treatment enhanced AAV particle purification.

FIG. 6 illustrates that examination of the post-product peaks demonstrated visible precipitate in the non-MNase treated samples produced either using the ExpiFectamine™ 293 Transfection Kit Enhancer or without the enhancer. However, MNase digestion prevented aggregation and precipitation of viral particles.

FIG. 7A-FIG. 7C illustrate that MNase treatment increased viral titers (FIG. 7A), genome copies/cell (FIG. 7B), and total genome copies (FIG. 7C), and reduced the amount of post-product produced, as compared to non-MNase treated samples and samples generated using the ExpiFectamine™ 293 Transfection Kit Enhancer.

FIG. 8A and FIG. 8B illustrate that an increase in genome copies per cell (GC/cell) (FIG. 8A), and total genome copies (FIG. 8B) was consistently observed in MNase treated samples (circle), relative to no MNase treated samples (square), for each of the three different elution buffers: citrate, low pH, and low/high pH.

FIG. 9A and FIG. 9B illustrate a summary of viral purification using the different purification conditions described in Table 3. FIG. 9A depicts a silver stain of the purified AAV particles. The three intense bands correspond with VP1, VP2, and VP3. FIG. 9B depicts a DNA agarose gel electrophoresis and the presence of the ITR-transgene contained within the AAV. C=citrate, pH 2.5; P=phosphoric acid, pH 1.5; “+¹”=pH 10.3; “+²”=pH 9.5; “+³”=pH 10.2; “+⁴”=pH 10.4; *=(C₂H₅)NCL anion exchange elution.

FIG. 10A-FIG. 10E illustrate results from static light scatter (SLS) and protein aggregation (Tagg) and melting (Tm) curves following no enzyme treatment (FIG. 10A); benzonase only (FIG. 10B); benzonase and MNase (FIG. 10C); benzonase, no MNase, high pH wash, and phosphoric acid elution (FIG. 10D); and benzonase, MNase, high pH wash, and phosphoric acid elution (FIG. 10E).

FIG. 11A and FIG. 11B illustrate the raw counts (FIG. 11A) and concentration results (FIG. 11B) using AlphaLISA to detect host-cell DNA following purification without benzonase or MNase (“no enzyme”); (2) purification with benzonase and citrate elution (“B, citrate (affinity)”); (3) purification with benzonase and citrate elution (“B, citrate”); (4) purification with benzonase, MNase, and phosphoric acid elution (“B, M, Phos”); (5) purification with benzonase, high pH (pH 10.3) wash, and phosphoric acid elution (“B, pH 10.3, Phos”); and (6) purification with benzonase, MNase, and phosphoric acid elution (“B, M, pH 10.3, Phos”).

FIG. 12 illustrates gel electrophoresis and silver-staining after collecting the AAV particle sample fraction corresponding with the product peak following Protocol #1 (lane 1), the AAV particle sample fraction corresponding with the product peak following Protocol #2 (lane 2), and the AAV particle sample fraction corresponding with the post-product peak following Protocol #2 (lane 3).

FIG. 13A-FIG. 13B illustrate that AAV particles are stable in the exemplary Formulation #2 (20 mM Tris, 100 mM NaCl, 5% Trehalose, 0.001% Poloxamer-188, pH 7.5) following 1 or more freeze/thaw cycles, as measured by DLS. FIG. 13A depicts minimal variation in the Z-average size and FIG. 13B depicts minimal variation in the polydispersity index (PDI).

FIG. 14A-FIG. 14B illustrate that AAV particles are stable in the exemplary Formulation #2 (20 mM Tris, 100 mM NaCl, 5% Trehalose, 0.001% Poloxamer-188, pH 7.5) following 1 to 3 months at −80° C., as measured by DLS. FIG. 14A depicts minimal variation in the Z-average size and FIG. 14B depicts minimal variation in the polydispersity index (PDI).

FIG. 15A-FIG. 15B illustrate that the exemplary Formulation #2 (20 mM Tris, 100 mM NaCl, 5% Trehalose, 0.001% Poloxamer-188, pH 7.5) has minimal AAV aggregation as measured by SEC-FLD following 1 to 5 freeze/thaw cycles. FIG. 15A shows very few high molecular weight species (HMWS) and FIG. 15B shows the measured samples were nearly all monomers.

FIG. 16A-FIG. 16B illustrate that the exemplary Formulation #2 (20 mM Tris, 100 mM NaCl, 5% Trehalose, 0.001% Poloxamer-188, pH 7.5) has minimal AAV aggregation as measured by SEC-FLD following 1 to 3 months at −80° C. FIG. 16A shows very few high molecular weight species (HMWS) and FIG. 16B shows the measured samples were nearly all monomers.

### 5. DETAILED DESCRIPTION

The present disclosure is based, in part, on the discovery that purified viral particles (e.g., AAV particles) that are substantially free of impurities (e.g., product-related impurities and process-related impurities) can be combined with one or more of a buffering agent, a cryoprotectant, a non-ionic surfactant, and optionally a pharmaceutically acceptable salt, to produce pharmaceutical compositions that enhance the stability and reduce or prevent aggregation of the purified AAV particles. In certain aspects, the present disclosure is also based, in part, on the discovery that the pharmaceutical compositions described herein are also suitable for enhancing the stability and limiting or preventing aggregation of high titer purified viral particles (e.g., concentrations greater than 1×10¹³ vg/mL).

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification. All patents, published patent applications and publications cited herein are incorporated by reference as if set forth fully herein.

It must be noted that as used herein and in the appended claims, the singular forms “a,” “an,” and “the” include plural reference unless the context clearly dictates otherwise.

Throughout this specification and the claims that follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein “consisting of” excludes any element, step, or ingredient not specified in the claim element. When used herein, “consisting essentially of” does not exclude materials or steps that do not materially affect the basic and novel characteristics of the claim. Any of the aforementioned terms of “comprising”, “containing”, “including”, and “having”, whenever used herein in the context of an aspect or embodiment of the application can be replaced with the term “consisting of” or “consisting essentially of” to vary scopes of the disclosure.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “adeno-associated virus (AAV)” is intended to mean both naturally occurring, including all the different AAV serotypes, as well as non-naturally occurring forms of AAV (e.g., recombinant rAAV, and pseudotypes), and variants thereof. AAV viruses consist of the Rep gene (translated as Rep78, Rep68, Rep52, Rep40—required for the AAV life cycle), and the Cap gene (translated as VP1, VP2, VP3—capsid proteins).

As used herein, the term “viral particle” or “AAV particle,” is intended to mean genome-containing (also known as “full capsids” or “partially-full capsids”), as well as empty capsids, or any combination of full, partially-full, and empty capsids, unless specified otherwise.

As used here, the term “impurity” or “impurities” is intended mean any component present in or with the purified AAV particles that is not the desired product, or an intended component of the pharmaceutical composition. AAV particle impurities include both process-related impurities and/or product-related impurities. Exemplary process-related impurities include, but are not limited to, residual host-cell components (e.g., proteins, DNA—including extra-viral, chromatin-associated DNA—and/or RNA), residual viral production components (e.g., plasmid DNA, and/or helper viruses), residual cell culture components (e.g., antibiotics, supplements, inducers, and/or growth factors), and residual purification components (e.g., buffers, inorganic salts, enzymes, media, and/or detergents), as well as other contaminants. Exemplary product-related impurities can include, but are not limited to, empty capsids (where undesirable), aggregated AAV particles, and degraded AAV particles. It is understood that a sample can be “substantially free” of one or more impurities, but continue to have a small amount (e.g., undetectable level, or below an acceptable range) of one or more impurities, and that “substantially free” does not require complete removal of all impurities.

As used herein, the term “titer” is intended to mean the quantity of virus in a given volume. A viral titer can include a “physical titer” or a “functional titer.” The physical titer is a measurement of how much virus is present, and is generally expressed as the number of viral particles per mL (VP/mL), or genome copies per mL (GC/mL). Functional titer, or infectious titer, is the measurement of how much virus actually infects a target cell and is generally expressed in the form of transduction units per mL (TU/mL), or for adenovirus as plaque-forming units per mL (pfu/mL) or infectious units per mL (ifu/mL). It is understood that functional titer will generally be lower than physical titer, usually by a factor of about 10 to about 100-fold.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the pharmaceutically acceptable compositions disclosed herein, include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride and mesylate salts.

As used herein, the term “sufficient amount” is intended to mean a quantity that is able to produce a desired effect or achieve a desired result, such as for example, binding of AAV particles to a solid support, such as an affinity support, or removing impurities (e.g., host-cell proteins, chromatin, and/or nucleic acids) from a sample containing AAV particles.

As used herein, the term “substantially free” when used in reference to a sample of AAV particles is intended to mean that the sample of AAV particles includes less than about 50%, less than about 20%, less than about 10%, or less than about 5% of an impurity, as compared to unpurified AAV particles (e.g., crude harvest).

Unless otherwise stated, any numerical value, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1 mg/mL to 10 mg/mL includes 0.9 mg/mL to 11 mg/mL. As used herein, the use of a numerical range expressly includes all possible subranges, all individual numerical values within that range, including integers within such ranges and fractions of the values unless the context clearly indicates otherwise.

In an attempt to help the reader of the application, the description has been separated in various paragraphs or sections, or is directed to various embodiments of the application. These separations should not be considered as disconnecting the substance of a paragraph or section or embodiments from the substance of another paragraph or section or embodiments. To the contrary, one skilled in the art will understand that the description has broad application and encompasses all the combinations of the various sections, paragraphs and sentences that can be contemplated. The discussion of any embodiment is meant only to be exemplary and is not intended to suggest that the scope of the disclosure, including the claims, is limited to these examples. The application contemplates use of any of the applicable components in any combination, whether or not a particular combination is expressly described.

5.1 Pharmaceutical Compositions

Provided herein are pharmaceutical compositions comprising a purified AAV particle, and one or more of a buffering agent, a cryoprotectant, a non-ionic surfactant, and optionally a pharmaceutically acceptable salt, where the AAV particle is substantially free of impurities. Also provided herein are methods for making the pharmaceutical compositions described herein.

5.1.1 AAV Particles

The present disclosure is based, in part, on the discovery that product purity can impact formulation development. Specifically, unencapsidated nucleic acids (e.g., host-cell DNA/RNA, plasmid DNA, etc.) and protein impurities [e.g., host-cell protein, empty capsids (where undesirable), etc.] can have profound impact on product stability and aggregation in addition to their obvious safety risks. Further, differential product purity can confound formulation results. This can manifest as a result of poor process robustness as well as during the introduction of the final formulation (whether by tangential flow filtration, dialysis, spin filtration, etc.), resulting in a variable impurity profile.

Impurities can include process-related impurities (e.g., impurities that remain after AAV particle purification, such as host-cell DNA or host-cell proteins), as well as product-related impurities (e.g., AAV particles that are not the full, non-aggregated capsid). Thus, the pharmaceutical compositions provided herein relate to purified AAV particles that are substantially free of impurities.

In some embodiments, the AAV particles are substantially free of an impurity, where the impurity includes a process-related impurity, a product-related impurity, or a combination thereof. Process related impurities can include a residual host-cell component, a residual viral production component, a residual cell culture component, a residual purification component, or a combination thereof. For example, following purification of the AAV particles, residual amounts of nucleic acids and/or proteins from host-cell used to generate the AAV particles can remain with the purified AAV particles if appropriate steps are not taken to remove these impurities. Accordingly, in some embodiments, the AAV particles are substantially free of a host-cell protein, a host-cell DNA, a host-cell RNA, or a combination thereof.

Certain aspects of the present disclosure are also based on the discovery that extra-viral, chromatin-associated DNA is also an impurity present in purified AAV particles that can greatly affect the purity of the AAV particles, and that the exemplary purification methods described herein (e.g., in Section 5.4, Section 5.5. and/or in the Examples) significantly improve the purity of the AAV particles by using a chromatin-DNA nuclease. Accordingly, in some embodiments, the AAV particles are substantially free of an impurity, where the impurity includes an extra-viral, chromatin-associated DNA.

Impurities can also arise from residual viral production components used to support AAV particle production. For example, nucleic acids such as plasmid DNA, or helper virus DNA, used to generate the AAV particles in the host-cell system can also remain with the purified AAV particles if appropriate steps are not taken to remove these impurities. Purification methods for removing residual viral production include those described herein (e.g., in Section 5.4, Section 5.5. and/or in the Examples). Accordingly, in some embodiments, the AAV particles are substantially free of residual viral production components. In certain embodiments, the AAV particles are substantially free of a plasmid DNA, a helper virus, or a combination thereof.

The AAV particles of the present disclosure that are substantially free of impurities can also include AAV particles that are substantially free of residual cell culture components. For example, host-cells can be cultured in media that contains antibiotics, growth factors, bovine serum, an inducer or other supplements. The removal of these cell culture components is important to generate purified AAV particles. Thus, in some embodiments, the purified AAV particles are substantially free of an antibiotic, a supplement, an inducer, a growth factor, or a combination thereof.

Another source of impurity that can affect the purified AAV particles include the purification reagents used to purify the AAV particles. For example, an exemplary method for purifying AAV particles includes chromatography, and often involves washing with inorganic salts. Another exemplary method for purifying AAV particles involves centrifugation, and this exemplary method often utilizes density gradient medium for the separation and isolation of the AAV particles. Therefore, it is preferable to remove such residual purification components from the purified AAV particles that are included in the pharmaceutical compositions described here. Accordingly, in some embodiments, the purified AAV particles are substantially free of a residual purification component. In some embodiments, the residual purification component comprises a buffer, an inorganic salt, an enzyme, a detergent, a medium, or a combination thereof.

Product-related impurities represent another source of impurity that can have an effect on the pharmaceutical compositions described herein. Such product-related impurities include, for example, aggregated AAV particles, degraded AAV particles, and/or empty capsids (where undesirable). Accordingly, in some embodiments, the purified AAV particles are substantially free of a product-related impurity. In some embodiments, the product-related impurity comprises an empty capsid (where undesirable), an aggregated AAV particle, a degraded AAV particle, or a combination thereof. In some embodiments, the product-related impurity comprises an aggregated AAV particle, a degraded AAV particle, or a combination thereof. In specific embodiments, the product-related impurity comprises an aggregated AAV particle.

The present disclosure is also based, in part, on the discovery that the pharmaceutical compositions described herein are able to reduce or prevent aggregation of even AAV particles with concentrations greater than 1×10¹³ viral genomes per milliliter (vg/mL). To date, existing formulations for AAV particles have been premised on the understanding that high ionic strength (>200 mM) formulations are required for high titers of virus (i.e., titers greater than 1×10¹³ vg/mL). By way of example, a typical high ionic strength formula is approximately 500 mM. In contrast, as described herein, the pharmaceutical compositions of the present disclosure are suitable for high titers of AAV particles, and do not involve high ionic strength formulations. Although the present disclosure is suitable for high titers of AAV particles, it is understood that the pharmaceutical compositions described herein need not be high concentrations, and that the pharmaceutical compositions are also suitable for AAV particles with titers below 1×10¹³ vg/mL.

In some embodiments, the purified AAV particle titer is about 1×10¹⁰ viral genomes per milliliter (vg/mL) or greater. In some embodiments, the purified AAV particle titer is about 1×10¹¹ viral genomes per milliliter (vg/mL) or greater. In some embodiments, the purified AAV particle titer is about 1×10¹² viral genomes per milliliter (vg/mL) or greater. In some embodiments, the purified AAV particle titer is about 1×10¹³ viral genomes per milliliter (vg/mL) or greater. In some embodiments, the purified AAV particle titer is about 1×10¹⁴ viral genomes per milliliter (vg/mL) or greater. In some embodiments, the purified AAV particle titer is about 1×10¹⁵ viral genomes per milliliter (vg/mL) or greater.

The AAV particles described herein are not limited to a specific serotype, and any AAV serotype, as well as AAV variants are suitable with the pharmaceutical compositions described herein. For example, in some embodiments, the AAV is AAV1, AAV2, AAV3, AAV4, AAV5, AAV6, AAV7, AAV8, AAV9, or AAV10. In some embodiments, the AAV comprises or consists essentially of AAV1. In some embodiments, the AAV comprises or consists essentially of AAV2. In some embodiments, the AAV comprises or consists essentially of AAV3. In some embodiments, the AAV comprises or consists essentially of AAV4. In some embodiments, the AAV comprises or consists essentially of AAV5. In some embodiments, the AAV comprises or consists essentially of AAV6. In some embodiments, the AAV comprises or consists essentially of AAV7. In some embodiments, the AAV comprises or consists essentially of AAV8. In some embodiments, the AAV comprises or consists essentially of AAV9. In some embodiments, the AAV comprises or consists essentially of AAV10. In some embodiments, the AAV comprises or consists essentially of an AAV variant. In certain aspects of the present disclosure the AAV comprises a rAAV.

The AAV particles described herein are intended for use in any downstream application that is compatible with AAV particles. For example, in some embodiments, the AAV particles are suitable for use in gene therapy and the purified AAV particles have an AAV vector with a therapeutic gene. However, it is also understood that the AAV particles can also be useful as a vaccine. Further, the downstream applicants extend beyond therapeutic use. For example, the AAV particles describe herein can also be useful as an agonist, for imaging applications, or other non-therapeutic uses. In some embodiments, it may be desirable to have AAV particles that include empty capsids; AAV particles that include a mixture of full capsids and empty capsids; AAV particles that include a mixture of full capsids, partially-full capsids, and empty capsids; or AAV particles that include a mixture of partially-full capsids and empty capsids. In some embodiments, the AAV particles comprise empty capsids. In some embodiments, the AAV particles comprise full capsids. In some embodiments, the AAV particles comprise partially full capsids. In some embodiments, the AAV particles comprise a mixture of full capsids and empty capsids. In some embodiments, the AAV particles comprise a mixture of full capsids, partially-full capsids, and empty capsids. In some embodiments, the AAV particles comprise a mixture of partially-full capsids and empty capsids. Thus, the downstream applications are not limited by the pharmaceutical compositions described herein.

5.1.2 Buffering Agents

In some embodiments, the pharmaceutical composition disclosed herein comprises a buffering agent. In some embodiments, the buffering agent is a pharmaceutically acceptable buffering agent well known in the art. In some embodiments, the buffering agent is HEPES, Tris, Bicine, acetate, glutamate, lactate, maleate, tartrate, phosphate, citrate, carbonate, glycinate, histidine, glycine, lysine, arginine, succinate, HEPES (4-(2-hydroxyethyl)-1-piperazineethanesulfonic acid), MOPS (3-(N-morpholino) propanesulfonic acid), MES (2-(N-morpholino)ethanesulfonic acid) and triethanolamine buffer, and mixtures thereof. In some embodiments, the buffering agent is HEPES. In some embodiments, the buffering agent is Tris. In some embodiments, the buffering agent is Bicine. In some embodiments, the buffering agent is acetate. In some embodiments, the buffering agent is glutamate. In some embodiments, the buffering agent is lactate. In some embodiments, the buffering agent is maleate. In some embodiments, the buffering agent is tartrate. In some embodiments, the buffering agent is phosphate. In some embodiments, the buffering agent is citrate. In some embodiments, the buffering agent is carbonate. In some embodiments, the buffering agent is glycinate. In some embodiments, the buffering agent is histidine. In some embodiments, the buffering agent is glycine. In some embodiments, the buffering agent is lysine. In some embodiments, the buffering agent is arginine. In some embodiments, the buffering agent is succinate. In some embodiments, the buffering agent is HEPES (4-(2-hydroxyethyl)-1-piperazineethanesulfonic acid). In some embodiments, the buffering agent is MOPS (3-(N-morpholino) propanesulfonic acid). In some embodiments, the buffering agent is IVIES (2-(N-morpholino)ethanesulfonic acid). In some embodiments, the buffering agent is triethanolamine buffer.

In some embodiments, the buffering agent is Tris or Histidine, and mixtures thereof. In some embodiments, the buffering agent is Tris. In some embodiments, the buffering agent is Histidine. In some embodiments, the buffering agent is a mixture of Tris and Histidine.

In some embodiments, the buffering agent includes Tris HCl or L-Histidine HCl, or a mixture thereof. In some embodiments, the buffering agent includes L-Histidine HCl. In some embodiments, the buffering agent includes Tris HCl. In some embodiments, the buffering agent is Tris HCl. In some embodiments, the buffering agent is L-Histidine HCl.

In some embodiments, the buffering agent has a pH in the range of about 4.0 to about 9.0, about 6.0 to about 8.0, or about 7.3 to about 7.7. In some embodiments, the buffering agent has a pH in the range of about 4.0 to about 9.0. In some embodiments, the buffering agent has a pH in the range of about 6.0 to about 8.0. In some embodiments, the buffering agent has a pH in the range of about 7.3 to about 7.7. In one embodiment, the buffering agent has a pH of about 7.5.

In some embodiments, the pharmaceutical composition disclosed herein comprises about 0 mM to about 50 mM, about 0 mM to about 45 mM, about 0 mM to about 40 mM, about 0 mM to about 35 mM, about 0 mM to about 30 mM, about 0 mM to about 25 mM, about 0 mM to about 20 mM, about 5 to about 30 mM, about 10 to about 25 mM, or about 15 mM to about 20 mM of a buffering agent. In some embodiments, the pharmaceutical composition disclosed herein comprises about 0.5 mM, about 1 mM, about 2 mM, about 3 mM, about 4 mM, about 5 mM, about 6 mM, about 7 mM, about 8 mM, about 9 mM, about 10 mM, about 11 mM, about 12 mM, about 13 mM, about 14 mM, about 15 mM, about 16 mM, about 17 mM, about 18 mM, about 19 mM, or about 20 mM of a buffering agent. In some embodiments, the pharmaceutical composition disclosed herein comprises about 0 mM to about 50 mM of a buffering agent. In some embodiments, the pharmaceutical composition comprises about 0 mM to about 45 mM of a buffering agent. In some embodiments, the pharmaceutical composition comprises about 0 mM to about 40 mM of a buffering agent. In some embodiments, the pharmaceutical composition comprises about 0 mM to about 35 mM of a buffering agent. In some embodiments, the pharmaceutical composition comprises about 0 mM to about 30 mM of a buffering agent. In some embodiments, the pharmaceutical composition comprises about 0 mM to about 25 mM of a buffering agent. In some embodiments, the pharmaceutical composition comprises about 0 mM to about 20 mM of a buffering agent. In some embodiments, the pharmaceutical composition comprises about 5 to about 30 mM of a buffering agent. In some embodiments, the pharmaceutical composition comprises about 10 to about 25 mM of a buffering agent. In some embodiments, the pharmaceutical composition comprises about 15 mM to about 20 mM of a buffering agent. In some embodiments, the pharmaceutical composition disclosed herein comprises about 0.5 mM of a buffering agent. In some embodiments, the pharmaceutical composition comprises about 1 mM of a buffering agent. In some embodiments, the pharmaceutical composition comprises about 2 mM of a buffering agent. In some embodiments, the pharmaceutical composition comprises about 3 mM of a buffering agent. In some embodiments, the pharmaceutical composition comprises about 4 mM of a buffering agent. In some embodiments, the pharmaceutical composition comprises about 5 mM of a buffering agent. In some embodiments, the pharmaceutical composition comprises about 6 mM of a buffering agent. In some embodiments, the pharmaceutical composition comprises about 7 mM of a buffering agent. In some embodiments, the pharmaceutical composition comprises about 8 mM of a buffering agent. In some embodiments, the pharmaceutical composition comprises about 9 mM of a buffering agent. In some embodiments, the pharmaceutical composition comprises about 10 mM of a buffering agent. In some embodiments, the pharmaceutical composition comprises about 11 mM of a buffering agent. In some embodiments, the pharmaceutical composition comprises about 12 mM of a buffering agent. In some embodiments, the pharmaceutical composition comprises about 13 mM of a buffering agent. In some embodiments, the pharmaceutical composition comprises about 14 mM of a buffering agent. In some embodiments, the pharmaceutical composition comprises about 15 mM of a buffering agent. In some embodiments, the pharmaceutical composition comprises about 16 mM of a buffering agent. In some embodiments, the pharmaceutical composition comprises about 17 mM of a buffering agent. In some embodiments, the pharmaceutical composition comprises about 18 mM of a buffering agent. In some embodiments, the pharmaceutical composition comprises about 19 mM of a buffering agent. In one embodiment, the pharmaceutical composition disclosed herein comprises about 20 mM of a buffering agent.

5.1.3 Cryoprotectants

In some embodiments, the pharmaceutical composition disclosed herein further comprises a cryoprotectant. In some embodiments, the cryoprotectant is an organic solvent, a polyol, a polymer, a sugar, or a combination thereof. In some embodiments, the cryoprotectant is DMSO (dimethyl sulfoxide), ethylene glycol, glycerol, propylene glycol, (MPD) 2-methyl-2,4-pentanediol, glycerol-3-phosphate, diethyl glycol, triethylene glycol, a polyvynyl alcohol, PEG, hydroxyethyl starch, sorbitol, mannitol, lactose, sucrose, trehalose, or a combination thereof. In some embodiments, the cryoprotectant is an organic solvent. In some embodiments, the cryoprotectant is a polyol. In some embodiments, the cryoprotectant is a polymer. In some embodiments, the cryoprotectant is a sugar. In some embodiments, the cryoprotectant is DMSO (dimethyl sulfoxide). In some embodiments, the cryoprotectant is ethylene glycol. In some embodiments, the cryoprotectant is glycerol. In some embodiments, the cryoprotectant is propylene glycol. In some embodiments, the cryoprotectant is (MPD) 2-methyl-2,4-pentanediol. In some embodiments, the cryoprotectant is glycerol-3-phosphate. In some embodiments, the cryoprotectant is diethyl glycol. In some embodiments, the cryoprotectant is triethylene glycol. In some embodiments, the cryoprotectant is a polyvynyl alcohol. In some embodiments, the cryoprotectant is PEG. In some embodiments, the cryoprotectant is hydroxyethyl starch. In some embodiments, the cryoprotectant is sorbitol. In some embodiments, the cryoprotectant is mannitol. In some embodiments, the cryoprotectant is lactose. In some embodiments, the cryoprotectant is sucrose. In some embodiments, the cryoprotectant is trehalose. In one embodiment, the cryoprotectant includes a sugar. In some embodiments, the sugar is a monosaccharide. In some embodiments, the sugar is a disaccharide. In some embodiments, the sugar is a polysaccharide. In one such embodiment, the sugar is sucrose, trehalose, or a combination thereof. In one embodiment, the cryoprotectant is trehalose.

In some embodiments, the pharmaceutical composition disclosed herein comprises about 1% (w/v) to about 10% (w/v), about 3% (w/v) to about 8% (w/v), or about 4% (w/v) to about 6% (w/v) cryoprotectant. In some embodiments, the pharmaceutical composition disclosed herein comprises about 1% (w/v) to about 10% (w/v) cryoprotectant. In some embodiments, the pharmaceutical composition disclosed herein comprises about 3% (w/v) to about 8% (w/v) cryoprotectant. In some embodiments, the pharmaceutical composition disclosed herein comprises about 4% (w/v) to about 6% (w/v) cryoprotectant. In some embodiments, the pharmaceutical composition disclosed herein comprises about 1% (w/v), about 2% (w/v), about 3% (w/v), about 4% (w/v), about 5% (w/v), about 6% (w/v), about 7% (w/v), about 8% (w/v), about 9% (w/v), or about 10% (w/v) cryoprotectant. In one embodiment, the pharmaceutical composition disclosed herein comprises about 1% (w/v) cryoprotectant. In one embodiment, the pharmaceutical composition disclosed herein comprises about 2% (w/v) cryoprotectant. In one embodiment, the pharmaceutical composition disclosed herein comprises about 3% (w/v) cryoprotectant. In one embodiment, the pharmaceutical composition disclosed herein comprises about 4% (w/v) cryoprotectant. In one embodiment, the pharmaceutical composition disclosed herein comprises about 5% (w/v) cryoprotectant. In one embodiment, the pharmaceutical composition disclosed herein comprises about 6% (w/v) cryoprotectant. In one embodiment, the pharmaceutical composition disclosed herein comprises about 7% (w/v) cryoprotectant. In one embodiment, the pharmaceutical composition disclosed herein comprises about 8% (w/v) cryoprotectant. In one embodiment, the pharmaceutical composition disclosed herein comprises about 9% (w/v) cryoprotectant. In one embodiment, the pharmaceutical composition disclosed herein comprises about 10% (w/v) cryoprotectant.

5.1.4 Non-Ionic Surfactant

In some embodiments, the pharmaceutical composition disclosed herein further comprises a non-ionic surfactant. In some embodiments, the non-ionic surfactant is a copolymer. In some embodiments, the non-ionic surfactant is a poloxamer. A poloxamer is a nonionic triblock copolymer composed of a central hydrophobic chain of polyoxypropylene (poly(propylene oxide)) flanked by two hydrophilic chains of polyoxyethylene (poly(ethylene oxide)). Poloxamers are also known by the tradename Pluronic®. Because the lengths of the polymer blocks can be customized, many different poloxamers exist that have slightly different properties. For the generic term “poloxamer,” these copolymers are commonly named with the letter “P” (for poloxamer) followed by three digits, the first two digits×100 give the approximate molecular mass of the polyoxypropylene core, and the last digit×10 gives the percentage polyoxyethylene content (e.g., P407=Poloxamer with a polyoxypropylene molecular mass of 4,000 g/mol and a 70% polyoxyethylene content). In some embodiments, the poloxamer is P188, P237, P338, or P407. In one embodiment, the poloxamer is P188. In one embodiment, the poloxamer is P237. In one embodiment, the poloxamer is P338. In one embodiment, the poloxamer is P407. In some embodiments the non-ionic surfactant is a polyoxyethylene sorbitan esters surfactant (commonly referred to as the Tweens), such as PS-20 and PS-80; a copolymer of ethylene oxide (EO), a phospholipid such as phosphatidyl choline (lecithin); a polyoxyethylene fatty ether derived from lauryl, cetyl, stearyl and oleyl alcohols (known as Brij surfactants), such as triethyleneglycol monolauryl ether (Brij 30) or polyoxyethylene (23) lauryl ether (Brij 35); and a sorbitan ester (commonly known as the SPANs), such as sorbitan trioleate (Span 85) and sorbitan monolaurate. In one embodiment, the non-ionic surfactant is polysorbate 20 (PS-20), polysorbate 80 (PS-80), or Brij surfactant, or a combination thereof. In one embodiments, the non-ionic surfactant is a polysorbate. In one embodiment, the polysorbate is PS-20. In one embodiment, the polysorbate is PS-40. In one embodiment, the polysorbate is PS-60. In one embodiment, the polysorbate is PS-80. In one embodiment, the non-ionic surfactant is a Brii surfactant. In one embodiment, the non-ionic surfactant is a copolymer of EO. In one embodiment, the non-ionic surfactant is a phospholipid. In one embodiment, the non-ionic surfactant is a phosphatidyl choline (lecithin). In one embodiment, the non-ionic surfactant is a polyoxyethylene fatty ether derived from a lauryl alcohol. In one embodiment, the non-ionic surfactant is a polyoxyethylene fatty ether derived from a cetyl alcohol. In one embodiment, the non-ionic surfactant is a polyoxyethylene fatty ether derived from a stearyl alcohol. In one embodiment, the non-ionic surfactant is a polyoxyethylene fatty ether derived from an oleyl alcohol. In one embodiment, the non-ionic surfactant is a triethyleneglycol monolauryl ether (Brij 30). In one embodiment, the non-ionic surfactant is a polyoxyethylene (23) lauryl ether (Brij 35). In one embodiment, the non-ionic surfactant is a sorbitan ester. In one embodiment, the non-ionic surfactant is a sorbitan trioleate (Span 85). In one embodiment, the non-ionic surfactant is a sorbitan monolaurate.

In some embodiments, the pharmaceutical composition disclosed herein comprises about 0.0001% (w/v) to about 0.1% (w/v), about 0.0005% (w/v) to about 0.005% (w/v), about 0.00075% (w/v) to about 0.0025% (w/v) non-ionic surfactant. In some embodiments, the pharmaceutical composition disclosed herein comprises about 0.0001% (w/v) to about 0.1% (w/v) non-ionic surfactant. In some embodiments, the pharmaceutical composition disclosed herein comprises about 0.0005% (w/v) to about 0.005% (w/v) non-ionic surfactant. In some embodiments, the pharmaceutical composition disclosed herein comprises about 0.00075% (w/v) to about 0.0025% (w/v) non-ionic surfactant.

In some embodiments, the pharmaceutical composition comprises about 0.001% (w/v), about 0.0015% (w/v), about 0.002% (w/v), about 0.0025% (w/v), about 0.003% (w/v), about 0.0035% (w/v), about 0.004% (w/v), about 0.0045% (w/v), about 0.005% (w/v), about 0.0055% (w/v), about 0.006% (w/v), about 0.0065% (w/v), about 0.007% (w/v), about 0.0075% (w/v), about 0.008% (w/v), about 0.0085% (w/v), about 0.009% (w/v), about 0.0095% (w/v) non-ionic surfactant. In some embodiments, the pharmaceutical composition comprises about 0.001% (w/v) non-ionic surfactant. In some embodiments, the pharmaceutical compositions comprises about 0.0015% (w/v) non-ionic surfactant. In some embodiments, the pharmaceutical compositions comprises about 0.002% (w/v) non-ionic surfactant. In some embodiments, the pharmaceutical compositions comprises about 0.0025% (w/v) non-ionic surfactant. In some embodiments, the pharmaceutical compositions comprises about 0.003% (w/v) non-ionic surfactant. In some embodiments, the pharmaceutical compositions comprises about 0.0035% (w/v) non-ionic surfactant. In some embodiments, the pharmaceutical compositions comprises about 0.004% (w/v) non-ionic surfactant. In some embodiments, the pharmaceutical compositions comprises about 0.0045% (w/v) non-ionic surfactant. In some embodiments, the pharmaceutical compositions comprises about 0.005% (w/v) non-ionic surfactant. In some embodiments, the pharmaceutical compositions comprises about 0.0055% (w/v) non-ionic surfactant. In some embodiments, the pharmaceutical compositions comprises about 0.006% (w/v) non-ionic surfactant. In some embodiments, the pharmaceutical compositions comprises about 0.0065% (w/v) non-ionic surfactant. In some embodiments, the pharmaceutical compositions comprises about 0.007% (w/v) non-ionic surfactant. In some embodiments, the pharmaceutical compositions comprises about 0.0075% (w/v) non-ionic surfactant. In some embodiments, the pharmaceutical compositions comprises about 0.008% (w/v) non-ionic surfactant. In some embodiments, the pharmaceutical compositions comprises about 0.0085% (w/v) non-ionic surfactant. In some embodiments, the pharmaceutical compositions comprises about 0.009% (w/v) non-ionic surfactant. In some embodiments, the pharmaceutical compositions comprises about 0.0095% (w/v) non-ionic surfactant.

5.1.5 Pharmaceutically Acceptable Salts

As provided herein, the pharmaceutical composition of the present disclosure can also include a pharmaceutically acceptable salt. Accordingly, in some aspects of the present disclosure, the pharmaceutical composition includes a purified AAV particle, a pharmaceutically acceptable salt, a buffering agent, a cryoprotectant, and a non-ionic surfactant.

In some embodiments, the pharmaceutically acceptable salt is a metal salt and salt of ammonia or organic amines that are safe for administration to a subject (e.g., a human) in a drug formulation. Suitable pharmaceutically acceptable salts include, but are not limited to, sodium, potassium, magnesium, calcium, cesium, ammonium, triethylamine, guanidine and N-substituted guanidine salts, acetamidine and N-substituted acetamidine, pyridine, picoline, ethanolamine, triethanolamine, dicyclohexylamine, and N,N′-dib enzylethylenediamine salts. Pharmaceutically acceptable salts (of basic nitrogen centers) include, but are not limited to inorganic acid salts such as the hydrochloride, hydrobromide, sulfate, phosphate; organic acid salts such as trifluoroacetate and maleate salts; sultanates such as methanesulfonate, ethanesulfonate, benzenesulfonate, p-toluenesulfonate, camphor sultanate and naphthalenesulfonate; amino acid salts, such as arginate, alaninate, asparginate and glutamate; and carbohydrate salts such as gluconate and galacturonate. Other salts that may be used herein are well known in the art, see for example, Remington's Pharmaceutical Sciences, 18^{th} eds., Mack Publishing, Easton Pa. (1990) or Remington: The Science and Practice of Pharmacy, 19^{th} eds., Mack Publishing, Easton Pa. (1995), which are incorporated herein by reference in their entirety for all intent and purposes. In one embodiment, the pharmaceutically acceptable salt is a sodium salt, a magnesium salt, a calcium salt, a potassium salt, a phosphate salt, or a sulfate salt. In one embodiment, the pharmaceutically acceptable salt is a metal salt. In one embodiment, the pharmaceutically acceptable salt is a sodium salt. In some embodiments, the pharmaceutically acceptable salt is a magnesium salt. In some embodiments, the pharmaceutically acceptable salt is a calcium salt. In some embodiments, the pharmaceutically acceptable salt is a potassium salt. In some embodiments, the pharmaceutically acceptable salt is a phosphate salt. In some embodiments, the pharmaceutically acceptable salt is a sulfate salt. In one embodiment, the pharmaceutically acceptable salt is sodium chloride.

In some embodiments, the pharmaceutically acceptable salt concentration is about 1 mM to about 200 mM. In some embodiments, the pharmaceutically acceptable salt concentration is about 10 mM to about 150 mM. In some embodiments, the pharmaceutically acceptable salt concentration is about 1 mM to about 49 mM. In some embodiments, the pharmaceutically acceptable salt concentration is about 5 mM to about 45 mM. In some embodiments, the pharmaceutically acceptable salt concentration is about 7.5 mM to about 40 mM. In some embodiments, the pharmaceutically acceptable salt concentration is about 10 mM to about 30 mM. In some embodiments, the pharmaceutically acceptable salt concentration is less than about 200 mM, less than about 150 mM, less than about 100 mM, less than about 90 mM, less than about 80 mM, less than about 70 mM, less than about 60 mM, less than about 50 mM, less than about 40 mM, less than about 30 mM, less than about 20 mM, or less than about 10 mM. In some embodiments, the pharmaceutically acceptable salt concentration is less than about 200 mM. In some embodiments, the pharmaceutically acceptable salt concentration is less than about 150 mM. In some embodiments, the pharmaceutically acceptable salt concentration is less than about 100 mM. In some embodiments, the pharmaceutically acceptable salt concentration is less than about 90 mM. In some embodiments, the pharmaceutically acceptable salt concentration is less than about 80 mM. In some embodiments, the pharmaceutically acceptable salt concentration is less than about 70 mM. In some embodiments, the pharmaceutically acceptable salt concentration is less than about 60 mM. In some embodiments, the pharmaceutically acceptable salt concentration is less than about 50 mM. In some embodiments, the pharmaceutically acceptable salt concentration is less than about 40 mM. In some embodiments, the pharmaceutically acceptable salt concentration is less than about 30 mM. In some embodiments, the pharmaceutically acceptable salt concentration is less than about 20 mM. In some embodiments, the pharmaceutically acceptable salt concentration is less than about 10 mM. In some embodiments, the pharmaceutically acceptable salt concentration is about 200 mM, about 150 mM, about 100 mM, or about 50 mM. In some embodiments, the pharmaceutically acceptable salt concentration is about 150 mM. In some embodiments, the pharmaceutically acceptable salt concentration is about 100 mM. In some embodiments, the pharmaceutically acceptable salt concentration is about 10 mM.

5.1.6 pH Conditions

As provided herein, the pharmaceutical composition of the present disclosure can have a pH that is about 4.0 to about 9.0. In some embodiments, the pH of the pharmaceutical composition is about 7.0 to about 8.0. In some embodiments, the pH of the pharmaceutical composition is about 7.3 to about 7.7. In certain embodiments, the pH of the pharmaceutical composition is about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0. In some embodiments, the pH of the pharmaceutical composition is about 7.3 to about 7.7. In certain embodiments, the pH of the pharmaceutical composition is about 4.0. In certain embodiments, the pH of the pharmaceutical composition is about 4.1. In certain embodiments, the pH of the pharmaceutical composition is about 4.2. In certain embodiments, the pH of the pharmaceutical composition is about 4.3. In certain embodiments, the pH of the pharmaceutical composition is about 4.4. In certain embodiments, the pH of the pharmaceutical composition is about 4.5. In certain embodiments, the pH of the pharmaceutical composition is about 4.6. In certain embodiments, the pH of the pharmaceutical composition is about 4.7. In certain embodiments, the pH of the pharmaceutical composition is about 4.8. In certain embodiments, the pH of the pharmaceutical composition is about 4.9. In certain embodiments, the pH of the pharmaceutical composition is about 5.0. In certain embodiments, the pH of the pharmaceutical composition is about 5.1. In certain embodiments, the pH of the pharmaceutical composition is about 5.2. In certain embodiments, the pH of the pharmaceutical composition is about 5.3. In certain embodiments, the pH of the pharmaceutical composition is about 5.4. In certain embodiments, the pH of the pharmaceutical composition is about 5.5. In certain embodiments, the pH of the pharmaceutical composition is about 5.6. In certain embodiments, the pH of the pharmaceutical composition is about 5.7. In certain embodiments, the pH of the pharmaceutical composition is about 5.8. In certain embodiments, the pH of the pharmaceutical composition is about 5.9. In certain embodiments, the pH of the pharmaceutical composition is about 6.0. In certain embodiments, the pH of the pharmaceutical composition is about 6.1. In certain embodiments, the pH of the pharmaceutical composition is about 6.2. In certain embodiments, the pH of the pharmaceutical composition is about 6.3. In certain embodiments, the pH of the pharmaceutical composition is about 6.4. In certain embodiments, the pH of the pharmaceutical composition is about 6.5. In certain embodiments, the pH of the pharmaceutical composition is about 6.6. In certain embodiments, the pH of the pharmaceutical composition is about 6.7. In certain embodiments, the pH of the pharmaceutical composition is about 6.8. In certain embodiments, the pH of the pharmaceutical composition is about 6.9. In certain embodiments, the pH of the pharmaceutical composition is about 7.0. In certain embodiments, the pH of the pharmaceutical composition is about 7.1. In certain embodiments, the pH of the pharmaceutical composition is about 7.2. In certain embodiments, the pH of the pharmaceutical composition is about 7.3. In certain embodiments, the pH of the pharmaceutical composition is about 7.4. In certain embodiments, the pH of the pharmaceutical composition is about 7.5. In certain embodiments, the pH of the pharmaceutical composition is about 7.6. In certain embodiments, the pH of the pharmaceutical composition is about 7.7. In certain embodiments, the pH of the pharmaceutical composition is about 7.8. In certain embodiments, the pH of the pharmaceutical composition is about 7.9. In certain embodiments, the pH of the pharmaceutical composition is about 8.0. In certain embodiments, the pH of the pharmaceutical composition is about 8.1. In certain embodiments, the pH of the pharmaceutical composition is about 8.2. In certain embodiments, the pH of the pharmaceutical composition is about 8.3. In certain embodiments, the pH of the pharmaceutical composition is about 8.4. In certain embodiments, the pH of the pharmaceutical composition is about 8.5. In certain embodiments, the pH of the pharmaceutical composition is about 8.6. In certain embodiments, the pH of the pharmaceutical composition is about 8.7. In certain embodiments, the pH of the pharmaceutical composition is about 8.8. In certain embodiments, the pH of the pharmaceutical composition is about 8.9. In certain embodiments, the pH of the pharmaceutical composition is about 9.0. Intervening ranges of the above-referenced pH values are also contemplated.

5.1.7 State of Pharmaceutical Composition

The pharmaceutical composition provided herein can be either a liquid composition or a frozen composition. Accordingly, in some embodiments, the pharmaceutical composition is in a liquid state. In other embodiments, the pharmaceutical composition is in a solid or semi-solid state.

5.2 Pharmaceutical Compositions and Routes of Administration

In certain embodiments, the compositions disclosed herein are administered to a subject by one or more methods known to a person skilled in the art, such as parenterally, transmucosally, transdermally, intramuscularly, intravenously, intra-dermally, intra-nasally, subcutaneously, intra-peritonealy, and formulated accordingly. In one embodiment, compositions described herein are administered via epidermal injection, intramuscular injection, intravenous, intra-arterial, subcutaneous injection, or intra-respiratory mucosal injection of a liquid preparation. Liquid formulations for injection include solutions and the like.

5.3 Storage Conditions

In certain embodiments, the compositions disclosed herein are stored at ambient temperature, such as about 25° C. In some embodiments, the compositions disclosed herein are stored at less than 25° C. In some embodiments, the compositions disclosed herein are stored between about 0° C. to about 25° C. In some embodiments, the compositions are stored between 0° C. to 10° C. In some embodiments, the compositions are stored between about 2° C. to about 8° C. In some embodiments, the compositions are stored at about 4° C. In some embodiments, the compositions are stored below 0° C. In some embodiments, the compositions are stored between about −20° C. to about −80° C. In some embodiments, the compositions are stored at about −20° C. In some embodiments, the compositions are stored at about −70° C. In some embodiments, the compositions are stored at about −80° C.

As provided herein, the composition disclosed herein can maintain or enhance the stability of the purified AAV particles and/or decrease or prevent the aggregation after one of more free/thaw cycles. In some embodiments, the composition disclosed herein maintains the stability of the purified AAV particles. In some embodiments, the composition disclosed herein decreases the aggregation after one free/thaw cycle. In some embodiments, the composition disclosed herein decreases the aggregation after more than one free/thaw cycle. In some embodiments, the composition disclosed herein prevents the aggregation after one free/thaw cycle. In some embodiments, the composition disclosed herein prevents the aggregation after more than one free/thaw cycle.

In certain embodiments, the compositions are stored below 0° C., and the stability is maintained or enhanced after one or more freeze/thaw cycles. In certain embodiments, the compositions are stored at about −20° C., and the stability is maintained or enhanced after one or more freeze/thaw cycles. In certain embodiments, the compositions are stored at about −20° C., and the stability is maintained or enhanced after one or more freeze/thaw cycles. In certain embodiments, the compositions are stored at about −70° C., and the stability is maintained or enhanced after one or more freeze/thaw cycles. In certain embodiments, the compositions are stored at about −80° C., and the stability is maintained or enhanced after one or more freeze/thaw cycles. In some embodiments, the stability is maintained. In some embodiments, the stability is maintained after one freeze/thaw cycle. In some embodiments, the stability is maintained after more than one freeze/thaw cycle. In some embodiments, the stability is enhanced. In some embodiments, the stability is enhanced after one freeze/thaw cycle. In some embodiments, the stability is enhanced after more than one freeze/thaw cycle.

In certain embodiments, the compositions are stored below 0° C., and the stability is maintained or enhanced after three or more freeze/thaw cycles. In certain embodiments, the compositions are stored at about −20° C., and the stability is maintained or enhanced after three or more freeze/thaw cycles. In certain embodiments, the compositions are stored at about −20° C., and the stability is maintained or enhanced after three or more freeze/thaw cycles. In certain embodiments, the compositions are stored at about −70° C., and the stability is maintained or enhanced after three or more freeze/thaw cycles. In certain embodiments, the compositions are stored at about −80° C., and the stability is maintained or enhanced after three or more freeze/thaw cycles. In some embodiments, the stability is maintained. In some embodiments, the stability is maintained after three freeze/thaw cycles. In some embodiments, the stability is maintained after more than three freeze/thaw cycles. In some embodiments, the stability is enhanced. In some embodiments, the stability is enhanced after three freeze/thaw cycles. In some embodiments, the stability is enhanced after more than three freeze/thaw cycle.

In some embodiments, the compositions are stored below 0° C., and aggregation is decreased or prevented after one or more freeze/thaw cycles. In some embodiments, the compositions are stored at about −20° C., and aggregation is decreased or prevented after one or more freeze/thaw cycles. In some embodiments, the compositions are stored at about −70° C., and aggregation is decreased or prevented after one or more freeze/thaw cycles. In some embodiments, the compositions are stored at about −80° C., and aggregation is decreased or prevented after one or more freeze/thaw cycles. In some embodiments, the aggregation of the AAV particle is less than 5% after one or more freeze/thaw cycles. In some embodiments, the aggregation of the AAV particle is less than 2% after one or more freeze/thaw cycles. In some embodiments, the aggregation of the AAV particle is less than 1% after one or more freeze/thaw cycles. In some embodiments, the aggregation is decreased. In some embodiments, aggregation is decreased after one freeze/thaw cycle. In some embodiments, the aggregation is decreased after more than one freeze/thaw cycle. In some embodiments, the aggregation is prevented. In some embodiments, the aggregation is prevented after one freeze/thaw cycle. In some embodiments, the aggregation is prevented after more than one freeze/thaw cycle.

In some embodiments, the compositions are stored below 0° C., and aggregation is decreased or prevented after three or more freeze/thaw cycles. In some embodiments, the compositions are stored at about −20° C., and aggregation is decreased or prevented after three or more freeze/thaw cycles. In some embodiments, the compositions are stored at about −70° C., and aggregation is decreased or prevented after three or more freeze/thaw cycles. In some embodiments, the compositions are stored at about −80° C., and aggregation is decreased or prevented after three or more freeze/thaw cycles. In some embodiments, the aggregation of the AAV particle is less than 5% after three or more freeze/thaw cycles. In some embodiments, the aggregation of the AAV particle is less than 2% after three or more freeze/thaw cycles. In some embodiments, the aggregation of the AAV particle is less than 1% after three or more freeze/thaw cycles. In some embodiments, the aggregation is decreased. In some embodiments, aggregation is decreased after three freeze/thaw cycles. In some embodiments, the aggregation is decreased after more than three freeze/thaw cycles. In some embodiments, the aggregation is prevented. In some embodiments, the aggregation is prevented after three freeze/thaw cycles. In some embodiments, the aggregation is prevented after more than three freeze/thaw cycles.

5.4 Exemplary Formulations

Exemplary formulations disclosed herein include, but are not limited to, any one of the formulations described below or in the Examples. It should be understood, however, that the application is not limited to the precise embodiments described below, and that are provided by way of example.

In one embodiment, the pharmaceutical composition includes a pharmaceutical composition that includes a purified AAV particle, a buffering agent, a cryoprotectant, and a non-ionic surfactant, where (a) the purified AAV particle is substantially free of an impurity, (b) the buffering agent concentration is about 20 mM, (c) the cryoprotectant is about 5% (w/v) trehalose, and (d) the non-ionic surfactant is about 0.001% (w/v) poloxamer 188. In some embodiments, the buffering agent comprises Tris HCl. In some embodiments, the buffering agent comprises L-Histidine HCl.

In one embodiment, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 10 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

In one embodiment, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 25 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

In one embodiment, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 50 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

In another aspect, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 100 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

In another aspect, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 125 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

In another aspect, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 150 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

In another aspect, the pharmaceutical composition includes a pharmaceutical composition that includes a purified AAV particle, a pharmaceutically acceptable salt, a buffering agent, a cryoprotectant, and a non-ionic surfactant, where (a) the purified AAV particle is substantially free of an impurity, (b) the pharmaceutically acceptable salt concentration is about 200 mM sodium chloride, (c) the buffering agent concentration is about 20 mM, (d) the cryoprotectant is about 5% (w/v) trehalose, and (e) the non-ionic surfactant is about 0.001% (w/v) poloxamer 188. In some embodiments, the buffering agent comprises Tris HCl. In some embodiments, the buffering agent comprises L-Histidine HCl.

5.5 Purification of AAV Particles

The AAV particles can be purified using various methods known in the art. Generally, purification involves centrifugation, chromatography, or filtration, or possibly a combination thereof. In some embodiments, purification can include a two-step purification protocol, including, for example, two chromatographic steps or a combination of chromatography with ultracentrifugation/filtration. By way of example, a two-step purification protocol can include purification by affinity chromatography (e.g., using heparin affinity resin) followed by polishing on an ion-exchange column. Another illustrative two-step purification protocol can involve ultracentrifugation (e.g., iodixanol density ultracentrifugation) with subsequent chromatography (e.g., affinity chromatography, such as heparin affinity purification).

Filtration and/or centrifugation of the starting material used in the purification process can help to remove some of the bulk impurities, such as for example cell debris and/or cell fragments. Accordingly, in some embodiments, the supernatant is a clarified supernatant. In certain embodiments, filtration and/or centrifugation are performed prior to one or more additional steps of purification, such as for example, chromatographic purification. Various methods for clarifying the supernatant are known in the art. For example, a 0.2 μm filter can be used to clarify the supernatant. In specific embodiments, the clarified supernatant can be treated with Benzonase® prior to one or more additional steps of purification.

Centrifugation techniques for purification of AAV particles can include, for example, density gradient centrifugation, ultracentrifugation, or a combination thereof. An exemplary type of density gradient centrifugation can involve CsCl, which forms a density gradient when subjected to a strong centrifugal field. For example, when the viruses are centrifuged to equilibrium in a CsCl salt, they are separated from contaminants and collected in bands on the basis of their buoyant densities. In some embodiments, purification of AAV particles includes multiple CsCl gradient centrifugation steps. Another exemplary density medium for purification of AAV particles can include iodixanol. In some embodiments, purification of AAV particles can include a density gradient centrifugation (e.g., a discontinuous iodixanol gradient centrifugation) as a pre-purification step, followed by an affinity chromatography virus purification step, such as by a heparinized support matrix chromatography or ion-exchange chromatography.

In some embodiments, purification of AAV particles involves chromatography. For example, the purification of large-scale quantities of AAV particles generally involves some form of chromatography whereby molecules in solution (mobile phase) are separated based on differences in chemical or physical interaction with a stationary material (solid phase or solid support). An exemplary type of chromatography includes, for example, gel filtration (also called size-exclusion chromatography or SEC), which uses a porous resin material to separate molecules based on size (i.e., physical exclusion). Another illustrative type of chromatography is affinity chromatography (also called affinity purification).

In certain embodiments, the chromatography involves a chromatographic column. As disclosed herein, various types of chromatographic columns can be used to purify AAV particles. In certain embodiments, the chromatographic column is a monolith. A monolith is a chromatographic column having a single block of a homogenous stationary phase with many interconnected channels. The stationary phase of the monolith can be of various chemistries, allowing the purification of different kinds of biomolecules with different characteristics. However, it is understood that the column need not be a monolith, and that beads, porous particle-based columns and membrane adsorbers can also be used.

Affinity chromatography makes use of specific binding interactions between molecules, such as ligand binding to a target molecule or a specific ionic interaction with a target molecule. An illustrative type of affinity chromatography involves separating viral particles from protein and nucleic acid contaminants based on a reversible interaction between the viral capsid and a specific biological ligand or receptor coupled to a chromatographic matrix. In some embodiments, purification by affinity chromatography can include a negatively charged cellulose affinity medium cellulofine sulfate. An alternative affinity purification approach is based on the recognition of AAV particles (e.g., AAV2 particles) by a monoclonal antibody (e.g., A20), allowing separation of unassembled capsid proteins. Additional illustrative examples for affinity chromatography include heparin affinity.

In some embodiments, affinity chromatography can be specific to the AAV capsid serotype or pseudotype of the AAV particle that is being purified. For example, some AAV serotypes, such as for example AAV1, 4 and 5, bind heparin columns less efficiently. Accordingly, in some embodiments, the affinity matrix for capture of, for example, AAV5 particles can include a sialic acid-rich protein called mucin covalently coupled to CNBr-activated Sepharose. Alternatively, PDGFR-alpha and PDGFR-beta can be used as specific molecules for the capture of, for example, AAV5 particles.

In some embodiments, the chromatography is ion exchange chromatography. Ion exchange chromatography involves the separation of molecules according to the strength of their overall ionic interaction with a solid phase material. Purification by ion-exchange chromatography is based on the net charge of proteins on the exterior of the viral capsid. The net charge of the surface proteins depends on the pH of the exposed amino-acid groups.

One exemplary type of ion exchange chromatography is anion exchange chromatography, which is used to separate molecules based on their net surface charge. Anion exchange chromatography uses a positively charged ion exchange resin with an affinity for molecules having net negative surface charges. It is understood that the examples provided above are intended to be exemplary and are not intended to be exhaustive of the types of chromatography that could be used with the present disclosure.

As provided herein, one exemplary type of purification that can be employed in the process of purifying the AAV particles is affinity chromatography. In some embodiments, the affinity chromatography can involve a particular ligand that is chemically immobilized or “coupled” to a solid support (e.g., affinity support) so that when a complex mixture is passed over the column, those molecules having specific binding affinity to the ligand become bound. In other embodiments, the affinity chromatography can involve ionic interaction based on a specific net surface charge so that the molecules having a specific binding affinity to the solid support based on their net surface charge become bound.

In some embodiments, the affinity chromatography is an ionic exchange chromatography. As described previously, ion exchange chromatography separates molecules according to the strength of their overall ionic interaction with a solid phase material, such as an affinity support. In some embodiments, the ionic exchange chromatography is anion exchange chromatography. Anion exchange chromatography can be used to separate molecules based on their net surface charge. For example, anion exchange chromatography uses a positively charged ion exchange resin with an affinity for molecules having net negative surface charges.

As described above, chromatography generally involves molecules in solution (mobile phase) that are separated based on differences in chemical or physical interaction with a stationary material (solid phase or solid support). The various forms of chromatography can optionally also involve washes to remove the unwanted components from the solid support. Thus, in some embodiments, the methods provided herein involve washing the solid support. After the other sample components are washed away, the bound molecule is stripped from the support (i.e., eluted), resulting in its purification from the original sample.

Elution of the AAV particles can be eluted either by a linear gradient elution or by using a step isocratic elution. Often, a gradient elution may be used to optimize elution conditions. Once the elution profile of the protein of interest has been established and it is known at what ionic strength or pH a protein elutes, a step elution can be used to speed the purification process. Depending on the type of chromatography that is used to purify the AAV particles, the elution conditions involve a competitive ligand, or involve changing pH, ionic strength, or polarity. The target protein can be eluted in a purified and concentrated form. For example, for ion exchange chromatography, the end-product can be eluted in an order depending on their net surface charge. Samples with pI values closer to 7.5 will elute at a lower ionic strength, and samples with very low pI values will elute at a high salt concentration.

In some embodiments, the AAV particles can be eluted using a low pH buffer. In certain embodiments, a high pH buffer is used immediately prior to the use of the low pH buffer, termed “high/low pH buffer.” In specific embodiments, the low pH is about pH 2.5. In some embodiments, the low pH buffer is a citrate buffer, a glycine buffer, or a phosphoric acid buffer. In certain embodiments, the low pH buffer comprises a weak acid.

As provided herein, the addition of ethanol to the elution step can improve the recovery of virus from the ion exchange column. Accordingly, in some embodiments, the elution buffer can include ethanol. In some embodiments, the ethanol can be about 5% to about 40% ethanol. In some embodiments, the ethanol can be about 10% to about 30% ethanol. In some embodiments, the ethanol can be about 15% to about 25% ethanol. In specific embodiments, the ethanol can be about 20% ethanol.

Elution performed using a low pH buffer often requires the elution buffer to be immediately neutralized. Thus, in some embodiments, the elution further includes neutralizing the pH of the buffer. In specific embodiments, neutralizing comprises adding Bis-Tris-Propane (BTP). In certain embodiments, neutralizing comprises adding Tris. Because many chromatographic elution buffers used for Ad or AAV purification procedures are not suitable for in vivo manipulations, additional purification steps such as dialysis or concentration may be necessary. Therefore, in some embodiments, the purification also includes dialysis and/or concentration of the AAV viral particles.

Thus, in some embodiments, provided herein is a method for purifying AAV particles that includes (a) incubating a supernatant comprising AAV particles with a solid support for a sufficient amount of time to bind the AAV particles; (b) contacting the supernatant comprising AAV particles with a composition comprising a chromatin-DNA nuclease; and (c) eluting the purified AAV particles. It is understood that contacting with the chromatin-DNA nuclease can also be performed before the binding of the AAV particles. Accordingly, in some embodiments, provided herein is a method for purifying adeno-associated viral (AAV) particles that includes (a) contacting the supernatant comprising AAV particles with a composition comprising a chromatin-DNA nuclease; (b) incubating a supernatant comprising AAV particles with a solid support for a sufficient amount of time to bind the AAV particles; and (c) eluting the purified AAV particles. Similarly, it is also understood that the contacting with the chromatin-DNA nuclease need not be combined in the setting of a solid support and can be combined with any AAV particle purification technique known in the art. In certain embodiments, the purified AAV particles are subjected to one or more additional purifications to polish the AAV particles. For example, the particles can be purified by affinity chromatography and then polished by a different type of chromatography, such as anion exchange chromatography.

In some embodiments, the method further includes washing the solid support before eluting the sample. As described herein, the washing away of non-bound sample components from the support can be performed using appropriate buffers that maintain the binding interaction between target and ligand. The washing can remove some unbound contaminants. In some embodiments, nonspecific binding interactions can be minimized by adding low levels of detergent or by moderate adjustments to salt concentration in the binding and/or wash buffer.

As provided herein, in some embodiments, the purity of the AAV particles can be increased by washing with a high pH. For example, the bulk harvest can be purified by affinity chromatography and then washed with a high pH buffer to remove impurities. In some embodiments, the high pH wash is followed by on-column enzyme treatment with Benzonase® and/or a chromatin-DNA nuclease. In certain embodiments, the high pH wash buffer is greater than pH 9. In some embodiments, the high pH wash buffer is between pH 9.5 and pH 10.9. In other embodiments, the high pH wash buffer is pH 9.5. In some embodiments, the high pH wash buffer is pH 10.2. In some embodiments, the high pH wash buffer is pH 10.3. In some embodiments, the high pH wash buffer is pH 10.4.

In some embodiments, the chromatin-DNA nuclease is micrococcal nuclease (MNase) (EC 3.1.31.1). MNase isolated from Staphylococcus aureus is a phosphodiesterase with non-specific endo-exonuclease activity capable of digesting nucleic acids (DNA and/or RNA). MNase digests exposed nucleic acids within the linker region connecting two nucleosomes until it reaches an obstruction (nucleosome or other nucleic acid binding protein). MNase can be suitable for removing nucleic acids from cell lysates, releasing chromatin-bound proteins, whereas DNase preferentially cleaves nucleosome-depleted or “free” DNA. MNase digests double-stranded, single-stranded, circular and linear nucleic acids. In some embodiments, the concentration of the MNase in the supernatant is greater than 2.5 units/mL (U/mL). In certain embodiments, the concentration of the MNase in the supernatant is greater than 10 units/mL. In specific embodiments, the concentration of the MNase in the supernatant is about 30 units/mL to about 100 units/mL. In more specific embodiments, the concentration of the MNase in the supernatant is about 60 units/mL. In some embodiments, the MNase is a polypeptide having the activity of a MNase. In one embodiment, the MNase is present in a sufficient amount to digest chromatin associated with an AAV particle.

In one embodiment, the MNase is present in a sufficient amount to reduce AAV particle impurities. In one embodiment, the AAV particle impurities comprise one or more of a host-cell DNA, a host-cell protein, a chromatin-associated DNA, and a DNA binding protein. In one embodiment, the AAV particle impurities comprise macroscopic and microscopic impurities. In one embodiment, the DNA binding protein comprises a histone.

In one embodiment, the MNase is present in an amount sufficient to decrease an AAV particle post-product fraction, as measured by absorbance at 260 nm. In one embodiment, the MNase is present in an amount sufficient to decrease an AAV particle post-product fraction, as measured by absorbance at 280 nm.

In one embodiment, the MNase is present in an amount sufficient to produce purified AAV particles comprising a melting temperature (Tm) within less than about 10° C. of an aggregation temperature (Tagg), as measured by dynamic light scatter (DLS). In one embodiment, the MNase is present in an amount sufficient to produce purified AAV particles comprising a melting temperature (Tm) within less than about 5° C. of an aggregation temperature (Tagg), as measured by dynamic light scatter (DLS). In one embodiment, the MNase is present in an amount sufficient to produce purified AAV particles comprising a melting temperature (Tm) within less than about 2° C. of an aggregation temperature (Tagg), as measured by dynamic light scatter (DLS).

Determination of the length of time necessary for the chromatin-DNA nuclease to release DNA binding proteins extra-virally complexed to AAV particles and free nucleic acids are within the skillset of a person skilled in the art. In certain embodiments, the incubation is for about 10 minutes to about 1 hour. In some embodiments, the incubation is for about 20 minutes to about 40 minutes. In specific embodiments, the incubation is for about 30 minutes.

In some embodiments of the methods provided herein, the composition that includes a chromatin-DNA nuclease can also include a Benzonase® nuclease (an endonuclease from Serratia marcescens; Enzyme Commission (EC) Number 3.1.30.2). Benzonase® is a promiscuous endonuclease that can degrade accessible DNA and RNA (e.g., non-chromatin DNA). It attacks and degrades all forms of DNA and RNA (e.g., single stranded, double stranded, linear and circular) and is effective over a wide range of operating conditions. For example, it can digest native or heat-denatured DNA and RNA.

The addition of Benzonase® can help to remove nuclease-sensitive nucleic acids present in the crude sample, such as residual nucleic acids from the host production cell. Although the addition of Benzonase® can be included to digest free nucleic acid, such as to reduce viscosity in protein samples, by itself it is insufficient to release DNA binding proteins extra-virally complexed to AAV particles. In some embodiments, the Benzonase® and the chromatin-DNA nuclease are incubated together. In specific embodiments, the Benzonase® and the chromatin-DNA nuclease are incubated together after affinity exchange chromatography. However, it is understood that the Benzonase® treatment need not be performed simultaneously with a chromatin-DNA nuclease. For example, in some embodiments, the Benzonase® is added to the bulk harvest before purification. It is further understood that any Benzonase® product is suitable with the present disclosure.

Although the present disclosure describes the use of Benzonase® as an exemplary endonuclease in certain embodiments, it is understood that any nuclease capable of reducing residual host cell DNA or a polypeptide having the activity of a nuclease capable of reducing residual host cell DNA can be used. Such alternative nucleases can include, for example, a cryonase (a recombinant endonuclease originating from a psychrophile, Shewanella sp.), a salt active nuclease (SAN), or DNase I™.

As provided herein, contacting sample containing AAV particles with MNase can remove chromatin-associated DNA, host cell proteins, and improve the overall yield of the AAV particles, relative to non-MNase treated samples. Therefore, in some embodiments, the purified AAV particles prepared using the methods provided herein are substantially free of chromatin-associated DNA, when compared to non-MNase contacted purified AAV particles. In certain embodiments, the purified AAV particles are substantially free of host cell proteins, when compared to non-MNase contacted purified AAV particles. In some embodiments, the AAV particles have an increased yield, when compared to non-MNase contacted purified AAV particles.

5.6 Production of AAV

Various production platforms are currently in use for the production of AAV particles and are known in the art, each of which is suitable for use in the purification methods and compositions described herein. Exemplary methods for the generation of AAV particles at large scale can involve, for example, plasmid DNA transfection in mammalian cells, Ad infection of stable mammalian cell lines, infection of mammalian cells with recombinant herpes simplex viruses (rHSVs), and infection of insect cells with recombinant baculoviruses (see, e.g., Penaud-Budloo M. et al., Mol Ther Methods Clin Dev. 2018 Jan. 8; 8:166-180).

An exemplary method for the production of AAV particles is, for example, the plasmid transfection of human embryonic HEK293 cells. For example, HEK293 cells can be simultaneously transfected with a plasmid containing the gene of interest and one or two helper plasmids, using either inorganic compounds (e.g. calcium phosphate) or organic compounds (e.g. polyethyleneimine (PEI)), or non-chemical (e.g. electroporation). The helper plasmid(s) allow the expression of the four Rep proteins (Rep78, Rep68, Rep52, Rep40), the three AAV structural proteins (VP1, VP2, and VP3), the AAP, and the adenoviral auxiliary functions E2A, E4, and VA RNA. The additional adenoviral E1A/E1B co-factors necessary for AAV replication can be expressed in HEK293 producer cells. The plasmids can be produced by conventional techniques in E. coli using bacterial origin and antibiotic-resistance gene or by minicircle (MC) technology. The producer cells, such as HEK293 producer cells, can be adherent or suspension cultures.

Another illustrative method of production involves infection of mammalian cells with rHSV vectors. Cells, such as the hamster BHK21 cell line or HEK293 and derivatives, can be infected with two rHSVs, one carrying the gene of interest bracketed by AAV ITR (rHSV-AAV) and the second with the AAV rep and cap ORFs of the desired serotype (rHSV-repcap) for the production of AAV particles.

Stable producer cell lines for AAV particle production offer a further illustrative method for the production of AAV particles. For example, stable producer cell lines can be derived from a cell line (e.g., HEK293 cells, HeLa cells, or a derivative) and engineered by introducing either the AAV rep and cap genes (packaging cell lines) and/or the AAV genome (e.g., rAAV genome) to be produced (producer cells). Another illustrative stable cell line for the production of AAV particles includes a stable cell line that incorporates the usually-toxic AAV replication (rep) gene as well as an AAV capsid (cap) gene and a transgene (see, e.g., U.S. Application No. 62/877,508, which is disclosed herein in its entirety).

It is further understood that the producer cell line need not be a mammalian cell line, and that non-mammalian cells, such as insect cells, and yeast can be used for the production. An illustrative example of a non-mammalian platform suitable for production of AAV particles includes the baculovirus-Sf9 insect-cell platform. The non-mammalian cell line suitable for production of AAV particles can be generated using transfection methods or as a stable cell line (see, e.g., Mietzsch M. et al. Hum. Gene Ther. 2014; 25: 212-222; Mietzsch M. et al. Hum. Gene Ther. Methods. 2017; 28: 15-22). The examples of AAV particle production platforms described above are understood to be illustrative, and not intended to be limiting, and that any of the various production platforms can be combined with the purification methods and compositions described herein.

As used herein, a “vector” is a nucleic acid molecule used to carry genetic material into a cell, where it can be replicated and/or expressed. Any vector known to those skilled in the art in view of the present disclosure can be used. Examples of vectors include, but are not limited to, plasmids, viral vectors (bacteriophage, animal viruses, and plant viruses), cosmids, and artificial chromosomes (e.g., YACs). Preferably, a vector is a DNA plasmid. One of ordinary skill in the art can construct a vector of the application through standard recombinant techniques in view of the present disclosure.

A vector of the application can be an expression vector. As used herein, the term “expression vector” refers to any type of genetic construct comprising a nucleic acid coding for an RNA capable of being transcribed. Expression vectors include, but are not limited to, vectors for recombinant protein expression, such as a DNA plasmid or a viral vector, and vectors for delivery of nucleic acid into a subject for expression in a tissue of the subject, such as a DNA plasmid or a viral vector. It will be appreciated by those skilled in the art that the design of the expression vector can depend on such factors as the choice of the host-cell to be transformed, the level of expression of protein desired, etc.

In some embodiments of the application, a vector is a non-viral vector. Examples of non-viral vectors include, but are not limited to, DNA plasmids, bacterial artificial chromosomes, yeast artificial chromosomes, bacteriophages, etc. Preferably, a non-viral vector is a DNA plasmid. A “DNA plasmid”, which is used interchangeably with “DNA plasmid vector,” “plasmid DNA” or “plasmid DNA vector,” refers to a double-stranded and generally circular DNA sequence that is capable of autonomous replication in a suitable host-cell. DNA plasmids used for expression of an encoded polynucleotide typically comprise an origin of replication, a multiple cloning site, and a selectable marker, which for example, can be an antibiotic resistance gene. Examples of DNA plasmids suitable that can be used include, but are not limited to, commercially available expression vectors for use in well-known expression systems (including both prokaryotic and eukaryotic systems), such as pSE420 (Invitrogen, San Diego, Calif.), which can be used for production and/or expression of protein in Escherichia coli; pYES2 (Invitrogen, Thermo Fisher Scientific), which can be used for production and/or expression in Saccharomyces cerevisiae strains of yeast; MAXBAC complete baculovirus expression system (Thermo Fisher Scientific), which can be used for production and/or expression in insect cells; pcDNA™ or pcDNA3™ (Life Technologies, Thermo Fisher Scientific), which can be used for high level constitutive protein expression in mammalian cells; and pVAX or pVAX-1 (Life Technologies, Thermo Fisher Scientific), which can be used for high-level transient expression of a protein of interest in most mammalian cells. The backbone of any commercially available DNA plasmid can be modified to optimize protein expression in the host-cell, such as to reverse the orientation of certain elements (e.g., origin of replication and/or antibiotic resistance cassette), replace a promoter endogenous to the plasmid (e.g., the promoter in the antibiotic resistance cassette), and/or replace the polynucleotide sequence encoding transcribed proteins (e.g., the coding sequence of the antibiotic resistance gene), by using routine techniques and readily available starting materials. (See e.g., Sambrook et al., Molecular Cloning a Laboratory Manual, Second Ed. Cold Spring Harbor Press (1989)).

Preferably, a DNA plasmid is an expression vector suitable for protein expression in mammalian host-cells. Expression vectors suitable for protein expression in mammalian host-cells include, but are not limited to, pUC, pcDNATM, pcDNA3TM, pVAX, pVAX-1, ADVAX, NTC8454, etc. For example, the vector can be based on pUC57, containing a pUC origin of replication and ampicillin resistance gene. It can further comprise a mammalian puromycin resistance gene cassette constructed from the Herpes virus thymidine kinase gene promoter, the puromycin N-acetyl transferase coding region, and a polyadenylation signal from bovine growth hormone gene. The vector can also comprise an Epstein Barr Virus (EBV) OriP replication origin fragment, which represents a composite of the ‘Dyad Symmetry’ region and the ‘Family of Repeats’ region of EBV.

A vector of the application can also be a viral vector. In general, viral vectors are genetically engineered viruses carrying modified viral DNA or RNA that has been rendered non-infectious, but still contains viral promoters and transgenes, thus allowing for translation of the transgene through a viral promoter. Because viral vectors are frequently lacking infectious sequences, they require helper viruses or packaging lines for large-scale transfection. Examples of viral vectors that can be used include, but are not limited to, adenoviral vectors, adeno-associated virus vectors, pox virus vectors, enteric virus vectors, Venezuelan Equine Encephalitis virus vectors, Semliki Forest Virus vectors, Tobacco Mosaic Virus vectors, lentiviral vectors, etc. The vector can also be a non-viral vector.

An illustrative viral vector is an adenovirus vector, e.g., a recombinant adenovirus vector. As used herein, the terms “recombinant adenovirus vector” and “recombinant adenoviral vector” and “recombinant adenoviral particles” are used interchangeably and refer to a genetically-engineered adenovirus that is designed to insert a polynucleotide of interest into a eukaryotic cell, such that the polynucleotide is subsequently expressed. Examples of adenoviruses that can be used as a viral vector of the invention include those having, or derived from, the serotypes Ad2, Ad5, Ad11, Ad12, Ad24, Ad26, Ad34, Ad35, Ad40, Ad48, Ad49, Ad50, Ad52 (e.g., RhAd52), and Pan9 (also known as AdC68); these vectors can be derived from, for example, human, chimpanzee (e.g., ChAd1, ChAd3, ChAd7, ChAd8, ChAd21, ChAd22, ChAd23, ChAd24, ChAd25, ChAd26, ChAd27.1, ChAd28.1, ChAd29, ChAd30, ChAd31.1, ChAd32, ChAd33, ChAd34, ChAd35.1, ChAd36, ChAd37.2, ChAd39, ChAd40.1, ChAd41.1, ChAd42.1, ChAd43, ChAd44, ChAd45, ChAd46, ChAd48, ChAd49, ChAd49, ChAd50, ChAd67, or SA7P), or rhesus adenoviruses (e.g., rhAd51, rhAd52, or rhAd53). A recombinant adenovirus vector can for instance be derived from a human adenovirus (HAdV, or AdHu), or a simian adenovirus such as chimpanzee or gorilla adenovirus (ChAd, AdCh, or SAdV) or rhesus adenovirus (rhAd).

Preferably, an adenovirus vector is a recombinant human adenovirus vector, for instance a recombinant human adenovirus serotype 5, or any one of recombinant human adenovirus serotype 26, 4, 35, 7, 48, etc. A recombinant viral vector useful for the application can be prepared using methods known in the art in view of the present disclosure. For example, in view of the degeneracy of the genetic code, several nucleic acid sequences can be designed that encode the same polypeptide. A polynucleotide encoding a protein of interest can optionally be codon-optimized to ensure proper expression in the host-cell (e.g., bacterial or mammalian cells). Codon-optimization is a technology widely applied in the art, and methods for obtaining codon-optimized polynucleotides will be well known to those skilled in the art in view of the present disclosure.

A non-naturally occurring nucleic acid molecule or a vector can comprise one or more expression cassettes. An “expression cassette” is part of a nucleic acid molecule or vector that directs the cellular machinery to make RNA and protein. An expression cassette can comprise a promoter sequence, an open reading frame, a 3′-untranslated region (UTR) optionally comprising a polyadenylation signal. An open reading frame (ORF) is a reading frame that contains a coding sequence of a protein of interest (e.g., Rep, Cap, recombinase or a recombinant protein of interest) from a start codon to a stop codon. Regulatory elements of the expression cassette can be operably linked to a polynucleotide sequence encoding a protein of interest.

A non-naturally occurring nucleic acid molecule or a vector of the application can contain a variety of regulatory sequences. A s used herein, the term “regulatory sequence” refers to any sequence that allows, contributes or modulates the functional regulation of the nucleic acid molecule, including replication, duplication, transcription, splicing, translation, stability and/or transport of the nucleic acid or one of its derivative (i.e., mRNA) into the host-cell or organism. Regulatory elements include, but are not limited to, a promoter, an enhancer, a polyadenylation signal, translation stop codon, a ribosome binding element, a transcription terminator, selection markers, origin of replication, etc.

A non-naturally occurring nucleic acid molecule or a vector can comprise a promoter sequence, preferably within an expression cassette, to control expression of a protein of interest. The term “promoter” is used in its conventional sense and refers to a nucleotide sequence that initiates the transcription of an operably linked nucleotide sequence. A promoter is located on the same strand near the nucleotide sequence it transcribes. Promoters can be a constitutive, inducible, or repressible. Promoters can be naturally occurring or synthetic. A promoter can be derived from sources including viral, bacterial, fungal, plants, insects, and animals. A promoter can be a homologous promoter (i.e., derived from the same genetic source as the vector) or a heterologous promoter (i.e., derived from a different vector or genetic source). For example, if the vector to be employed is a DNA plasmid, the promoter can be endogenous to the plasmid (homologous) or derived from other sources (heterologous). Preferably, the promoter is located upstream of the polynucleotide encoding a protein of interest within an expression cassette.

Examples promoters that can be used include, but are not limited to, a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter (CMV-IE), Epstein Barr virus (EBV) promoter, or a Rous sarcoma virus (RSV) promoter. A promoter can also be a promoter from a human gene such as human actin, human myosin, human hemoglobin, human muscle creatine, or human metalothionein. A promoter can also be a tissue specific promoter, such as a muscle or skin specific promoter, natural or synthetic. Preferably, a promoter is a strong eukaryotic promoter, such as a cytomegalovirus (CMV) promoter (nt −672 to +15), EF1-alpha promoter, herpes virus thymidine kinase gene promoter, etc.

A non-naturally occurring nucleic acid molecule or a vector can comprise additional polynucleotide sequences that stabilize the expressed transcript, enhance nuclear export of the RNA transcript, and/or improve transcriptional-translational coupling. Examples of such sequences include polyadenylation signals and enhancer sequences. A polyadenylation signal is typically located downstream of the coding sequence for a protein of interest (e.g., Rep, Cap, recombinase) within an expression cassette of the vector. Enhancer sequences are regulatory DNA sequences that, when bound by transcription factors, enhance the transcription of an associated gene. An enhancer sequence is preferably downstream of a promoter sequence and can be downstream or upstream of a coding sequence within an expression cassette of the vector.

Any polyadenylation signal known to those skilled in the art in view of the present disclosure can be used. For example, the polyadenylation signal can be a SV40 polyadenylation signal, AAV2 polyadenylation signal (bp 4411-4466, NC 001401), a polyadenylation signal from the Herpes Simplex Virus Thymidine Kinase Gene, LTR polyadenylation signal, bovine growth hormone (bGH) polyadenylation signal, human growth hormone (hGH) polyadenylation signal, or human β-globin polyadenylation signal. Preferably, a polyadenylation signal is a bovine growth hormone (bGH) polyadenylation signal, the polyadenylation signal of AAV2 having nucleotide numbers 4411 to 4466 of the nucleotide sequence of GenBank accession number NC 001401, or a SV40 polyadenylation signal.

Any enhancer sequence known to those skilled in the art in view of the present disclosure can be used. For example, an enhancer sequence can be human actin, human myosin, human hemoglobin, human muscle creatine, or a viral enhancer, such as one from CMV, HA, RSV, or EBV. Examples of particular enhancers include, but are not limited to, Woodchuck HBV Post-transcriptional regulatory element (WPRE), intron/exon sequence derived from human apolipoprotein A1 precursor (ApoAI), untranslated R-U5 domain of the human T-cell leukemia virus type 1 (HTLV-1) long terminal repeat (LTR), a splicing enhancer, a synthetic rabbit β-globin intron, or any combination thereof.

Preferably, an enhancer sequence comprises a P5 promoter of an AAV. The P5 promoter is part of a cis-acting Rep-dependent element (CARE) inside the coding sequence of the rep gene. CARE was shown to augment the replication and encapsidation when present in cis. CARE is also important for amplification of chromosomally integrated rep genes (if AAV ITRs are not present) as in some AAV particle producer cell lines. While not wishing to be bound by theories, it is believed that a P5 promoter placed downstream of a cap coding sequence potentially act as an enhancer to increase Cap expression, thus AAV particle yields, and that it also provides enhancer activity for amplifying genes integrated into a chromosome.

A non-naturally occurring nucleic acid molecule or a vector, such as a DNA plasmid, can also include a bacterial origin of replication and an antibiotic resistance expression cassette for selection and maintenance of the plasmid in bacterial cells, e.g., E. coli. An origin of replication (ORI) is a sequence at which replication is initiated, enabling a plasmid to reproduce and survive within cells. Examples of ORIs suitable for use in the application include, but are not limited to ColE1, pMB1, pUC, pSC101, R6K, and 15A, preferably pUC.

Vectors for selection and maintenance in bacterial cells typically include a promoter sequence operably linked to an antibiotic resistance gene. Preferably, the promoter sequence operably linked to an antibiotic resistance gene differs from the promoter sequence operably linked to a polynucleotide sequence encoding a protein of interest. The antibiotic resistance gene can be codon optimized, and the sequence composition of the antibiotic resistance gene is normally adjusted to bacterial, e.g., E. coli, codon usage. Any antibiotic resistance gene known to those skilled in the art in view of the present disclosure can be used, including, but not limited to, kanamycin resistance gene (Kan^{r}), ampicillin resistance gene (Amp^{r}), and tetracycline resistance gene (Tet^{r}), as well as genes conferring resistance to chloramphenicol, bleomycin, spectinomycin, carbenicillin, etc.

5.7 Assays for Testing Pharmaceutical Composition Properties

As provided herein, the pharmaceutical compositions of the present disclosure are able to prevent aggregation and/or enhance the stability of the AAV particles. Various techniques are known in the art for measuring the physical properties (e.g., AAV particle size), viral titer, and/or purity of the AAV particles. Exemplary assays are provided below, and in some instances, the assay can measure multiple properties. It should be understood, however, that the application is not limited to the assays described below.

5.7.1 Exemplary Assays to Measure Identity

Various assays are known in the art for evaluating the identity of the AAV particle preparation. For example, assays that analyze viral protein expression can be useful in evaluating the identity of the AAV particles. Such assays included, but are not limed to, SDS-PAGE, mass spectrometry, immunoblotting, and ELISA. The proper number, molecular weight, and stoichiometry of the viral proteins can be used to positively identify the vector, as well as the presence of impurities. In addition, the vector genome can be evaluated using PCR or high throughput NGS (next generation genome sequencing) to ensure positive identity.

5.7.2 Exemplary Assays to Measure Viral Titer

The AAV particles of the present disclosure can also be characterized by their viral titers. Viral titers can include physical titers, as well as functional titers. Physical titer calculates the total number of alive and dead viral particles present and is expressed as the number of viral particles per mL (VP/mL), or for AAV as genome copies per mL (GC/mL). Various methods can be used to determine the physical titer of virus based on quantifying the concentration of viral genomes or viral proteins. Suitable techniques include, but are not limited to, DNA hybridization, real-time PCR—including, but not limited to, quantitative PCR (qPCR), and digital drop PCR (dPCR), optical density (A260/280), NanoSight, and high-performance liquid chromatography (HPLC).

For example, the Optical Density (A260/280) assay measures the concentration of viral DNA and protein. It is a physical assay measuring the concentration of viral particles (VP). HPLC is also a rapid method to quantify total viral particles by separation of intact virus particles from other cellular contaminants or virus particle fragments.

Functional titer measures how much virus gets into a target cell, and can include assessment of the number of colony forming units following antibiotic selection if the vector contains an antibiotic resistance gene, or, if the vector contains a fluorescent protein, flow cytometry or immunofluorescence analysis of the target cells. Alternatively, if the vector does not express a fluorescent protein, determining the number of integrated proviral DNA copies per cell by qPCR provides a fast and easy method for assessing functional titer.

5.7.3 Exemplary Assays to Measure Purity

Assays to measure the purity of AAV particles are known in the art. Traditional methods to determine residual DNA levels include, PicoGreen and DNA Threshold Assays. More recently, quantifying host cell nucleic acids has been done using real-time or quantitative PCR (qPCR). As provided herein, a further exemplary assay to measure residual host-cell DNA is an AlphaLISA Assay.

Detection of host cell-associated proteins can be performed by ELISA, where antibodies react with host proteins. Protein impurities can be also be successfully detected using transmission electron microscopy (TEM).

Additional exemplary methods for measuring purity include Mass Spectrometry (MS) and chromatography methods. In addition to being able to detect and identity protein contaminants, MS and chromatograph can also be used to identify detergents and organic solvents in the AAV particle preparation. The quality of AAV particle preparation can also be analyzed through direct visualization with TEM or analytical ultracentrifugation (AUC).

Analytical ultracentrifugation (AUC) is a powerful tool to distinguish and quantify different AAV species by either mass (sedimentation velocity) or density (sedimentation equilibrium). AUC is a technique that monitors the sedimentation of particles over time under a centrifugal field, providing critical information on particle molecular weight, homogeneity, and interactions with other particles and itself. The empty capsids have a different density and/or mass than the full particles and those partially filled, allowing baseline separation on the basis of hydrodynamics under centrifugal force. In some instruments, multi-wavelength absorbance can be used to quantify both genomic DNA and viral capsid content in a single experiment.

5.7.4 Exemplary Assays to Measure Stability

Exemplary techniques for measuring the stability of AAV particles include, but are not limited to, dynamic light scattering (DLS), analytical ultracentrifugation (AUC), light microscopy, size exclusion chromatography (SEC)—including SEC with multi-angle static light scattering (MALS), transmission electron microscopy (TEM), and field flow fractionation with multi-angle static light scattering (FFF-MALS).

DLS is a well-established analytical technique in the field of AAV development. Its primary use is to test for aggregate formation. Due to its high sensitivity towards large species, even small impurities caused by aggregation can be detected. It is also possible to combine DLS with SLS. In DLS, the hydrodynamic size and size distribution of particles in solution can be obtained. It may be of interest to examine this measurement as a function of time and temperature. For example, although at low temperatures a protein may be stable and show repeatable size (and scattering intensity) measurements, typically at some elevated temperature (Tagg), protein molecules will show a tendency to oligomerize or aggregate. The temperature at which this occurs will depend on the protein itself, plus the buffer composition. DLS or the combination of DLS and SLS therefore allows the instrument user to screen the melting (Tm), aggregation (Tagg) and onset temperatures (Tonset)—the temperature at which molecules have a tendency to aggregate together. This information can help to understand both the temperature dependence of the colloidal and conformational stability.

As provided herein, in some embodiments the pharmaceutical composition maintains or enhances the stability of the purified AAV particle. In some embodiments, the pharmaceutical composition reduces or prevents aggregation of the purified AAV particle. In some embodiments, the pharmaceutical composition (a) maintains or enhances the stability of the purified AAV particle; and (b) reduces or prevents aggregation of the purified AAV particle.

In certain embodiments, the stability is maintained or enhanced and/or aggregation is decreased or prevented after one of more free/thaw cycles. In some embodiments, the stability is maintained or enhanced after one or more freeze/thaw cycles. In some embodiments, the stability is maintained or enhanced after three or more freeze/thaw cycles.

In some embodiments, the aggregation of the AAV particle is less than 5% after one or more freeze/thaw cycles. In some embodiments, the aggregation of the AAV particle is less than 2% after one or more freeze/thaw cycles. In some embodiments, the aggregation of the AAV particle is less than 1% after one or more freeze/thaw cycles.

In some embodiments, the aggregation of the AAV particle is less than 5% after three or more freeze/thaw cycles. In some embodiments, the aggregation of the AAV particle is less than 2% after three or more freeze/thaw cycles. In some embodiments, the aggregation of the AAV particle is less than 1% after three or more freeze/thaw cycles.

Any of the assays described above can be used to measure the aggregation and/or stability. For example, in some embodiments, the aggregation and/or stability is determined by DLS. In some embodiments, the aggregation and/or stability is determined by AUC.

### 6. EMBODIMENTS

This invention provides the following non-limiting embodiments.

In one set of embodiments, provided are:
A1. A pharmaceutical composition comprising a purified adeno-associated virus (AAV) particle, a buffering agent, a cryoprotectant, and a non-ionic surfactant, wherein
   (a) the purified AAV particle is substantially free of an impurity,
   (b) the buffering agent concentration is about 0 mM to about 50 mM,
   (c) the cryoprotectant is about 1% to about 10% (w/v), and
   (d) the non-ionic surfactant is about 0.0001% (w/v) to about 0.1% (w/v).
A2. The pharmaceutical composition of embodiment A1, further comprising a pharmaceutically acceptable salt, wherein the pharmaceutically acceptable salt concentration is about 1 mM to about 200 mM.
A3. The pharmaceutical composition of embodiment A2, wherein the pharmaceutically acceptable salt is about 10 mM to about 150 mM.
A4. The pharmaceutical composition of embodiment A2 or A3, wherein the pharmaceutically acceptable salt concentration is about 10 mM.
A5. The pharmaceutical composition of embodiment A2 or A3, wherein the pharmaceutically acceptable salt concentration is about 100 mM.
A6. The pharmaceutical composition of embodiment A2 or A3, wherein the pharmaceutically acceptable salt concentration is about 150 mM.
A7. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 1 mM to about 49 mM,
   (c) the buffering agent concentration is about 0 mM to about 50 mM,
   (d) the cryoprotectant is about 1% to about 10% (w/v), and
   (e) the non-ionic surfactant is about 0.0001% (w/v) to about 0.1% (w/v).
A8. The pharmaceutical composition of embodiment A7, wherein the pharmaceutically acceptable salt concentration is about 5 mM to about 45 mM.
A9. The pharmaceutical composition of embodiment A7 or A8, wherein the pharmaceutically acceptable salt concentration is about 7.5 mM to about 40 mM.
A10. The pharmaceutical composition of any one of embodiments A7 to A9, wherein the pharmaceutically acceptable salt concentration is about 10 mM to about 30 mM.
A11. The pharmaceutical composition of any one of embodiments A7 to A10, wherein the pharmaceutically acceptable salt concentration is about 10 mM.
A12. The pharmaceutical composition of any one of embodiments A2 to A11, wherein the pharmaceutically acceptable salt is selected from the group consisting of a sodium salt, a magnesium salt, a calcium salt, a potassium salt, a phosphate salt, and a sulfate salt.
A13. The pharmaceutical composition of embodiment A12, wherein the sodium salt comprises sodium chloride.
A14. The pharmaceutical composition of any one of embodiments A1 to A13, wherein the buffering agent comprises Tris HCl.
A15. The pharmaceutical composition of any one of embodiments A1 to A13, wherein the buffering agent comprises L-Histidine HCl.
A16. The pharmaceutical composition of any one of embodiments A1 to A15, wherein the buffering agent concentration comprises about 20 mM.
A17. The pharmaceutical composition of any one of embodiments A1 to A16, wherein the cryoprotectant is about 3% (w/v) to about 8% (w/v).
A18. The pharmaceutical composition of any one of embodiments A1 to A17, wherein the cryoprotectant is about 4% (w/v) to about 6% (w/v).
A19. The pharmaceutical composition of any one of embodiments A1 to A18, wherein the cryoprotectant is about 5% (w/v).
A20. The pharmaceutical composition of any one of embodiments A1 to A19, wherein the cryoprotectant comprises a sugar.
A21. The pharmaceutical composition of embodiment A20, wherein the sugar comprises sucrose, trehalose, or a combination thereof.
A22. The pharmaceutical composition of embodiment A20, wherein the sugar comprises trehalose.
A23. The pharmaceutical composition of any one of embodiments A1 to A22, wherein the non-ionic surfactant is about 0.0005% (w/v) to about 0.005% (w/v).
A24. The pharmaceutical composition of any one of embodiments A1 to A23, wherein the non-ionic surfactant is about 0.00075% (w/v) to about 0.0025% (w/v).
A25. The pharmaceutical composition of any one of embodiments A1 to A24, wherein the non-ionic surfactant is about 0.001% (w/v).
A26. The pharmaceutical composition of any one of embodiments A1 to A25, wherein the non-ionic surfactant is selected from the group consisting of a copolymer, a polyoxyethylene sorbitan ester, a phospholipid, a Brij surfactant, and a sorbitan ester, or a combination thereof
A27. The pharmaceutical composition of embodiment A26, wherein the polyoxyethylene sorbitan ester is selected from the group consisting of (PS-20), and polysorbate 80 (PS-80), or a combination thereof.
A28. The pharmaceutical composition of embodiment A26, wherein the copolymer comprises a poloxamer.
A29. The pharmaceutical composition of embodiment A28, wherein the poloxamer is selected from the group consisting of poloxamer 188 (P188), poloxamer 237 (P237), poloxamer 338 (P338), and poloxamer 407 (P407), or a combination thereof.
A30. The pharmaceutical composition of embodiment A28, wherein the poloxamer comprises poloxamer 188 (P188).
A31. A pharmaceutical composition comprising a purified adeno-associated virus (AAV) particle, a buffering agent, a cryoprotectant, and a non-ionic surfactant, wherein
   (a) the purified AAV particle is substantially free of an impurity,
   (b) the buffering agent concentration is about 20 mM,
   (c) the cryoprotectant is about 5% (w/v) trehalose, and
   (d) the non-ionic surfactant is about 0.001% (w/v) poloxamer 188.
A32. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 10 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A33. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 25 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A34. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 50 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A35. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 100 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A36. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 125 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A37. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 150 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A38. A pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 200 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
A39. The pharmaceutical composition of any one of embodiments A31 to A38 wherein the buffering agent comprises Tris HCl.
A40. The pharmaceutical composition of any one of embodiments A31 to A38, wherein the buffering agent comprises L-Histidine HCl.
A41. The pharmaceutical composition of any one of embodiments A1 to A40, wherein the pharmaceutical composition pH is about 4.0 to about 9.0.
A42. The pharmaceutical composition of any one of embodiments A1 to A41, wherein the pharmaceutical composition pH is about 7.0 to about 8.0.
A43. The pharmaceutical composition of any one of embodiments A1 to A42, wherein the pharmaceutical composition pH is about 7.3 to about 7.7.
A44. The pharmaceutical composition of any one of embodiments A1 to A43, wherein the pharmaceutical composition pH is about 7.5.
A45. The pharmaceutical composition of any one of embodiments A1 to A44, wherein the AAV is selected from the group consisting of AAV1, AAV2, AAV3, AAV4, AAV5, AAV6, AAV7, AAV8, AAV9, and AAV10.
A46. The pharmaceutical composition of any one of embodiments A1 to A45, wherein the AAV comprises a recombinant AAV (rAAV).
A47. The pharmaceutical composition of any one of embodiments A1 to A46, wherein the purified AAV particle titer is about 1×10¹⁰ viral genomes per milliliter (vg/mL) or greater.
A48. The pharmaceutical composition of any one of embodiments A1 to A46, wherein the purified AAV particle titer is about 1×10¹¹ viral genomes per milliliter (vg/mL) or greater.
A49. The pharmaceutical composition of any one of embodiments A1 to A46, wherein the purified AAV particle titer is about 1×10¹² viral genomes per milliliter (vg/mL) or greater.
A50. The pharmaceutical composition of any one of embodiments A1 to A46, wherein the purified AAV particle titer is about 1×10¹³ viral genomes per milliliter (vg/mL) or greater.
A51. The pharmaceutical composition of any one of embodiments A1 to A46, wherein the purified AAV particle titer is about 1×10¹⁴ viral genomes per milliliter (vg/mL) or greater.
A52. The pharmaceutical composition of any one of embodiments A1 to A46, wherein the purified AAV particle titer is about 1×10¹⁵ viral genomes per milliliter (vg/mL) or greater.
A53. The pharmaceutical composition of any one of embodiments A1 to A52, wherein the impurity comprises a process-related impurity, a product-related impurity, or a combination thereof.
A54. The pharmaceutical composition of embodiment A53, wherein the process-related impurity is selected from the group consisting of a residual host-cell component, a residual viral production component, a residual cell culture component, a residual purification component, or a combination thereof.
A55. The pharmaceutical composition of embodiment A54, wherein the residual host-cell component comprises a host-cell protein, a host-cell DNA, a host-cell RNA, or a combination thereof
A56. The pharmaceutical composition of embodiment A55, wherein the host-cell DNA comprises an extra-viral, chromatin-associated DNA.
A57. The pharmaceutical composition of embodiment A56, wherein the residual viral production component comprises a plasmid DNA, a helper virus, or a combination thereof
A58. The pharmaceutical composition of embodiment A54, wherein the residual cell culture component comprises an antibiotic, a supplement, an inducer, a growth factor, or a combination thereof.
A59. The pharmaceutical composition of embodiment A54, wherein the residual purification component comprises a buffer, an inorganic salt, an enzyme, a detergent, a medium, or a combination thereof.
A60. The pharmaceutical composition of embodiment A53, wherein the product-related impurity comprises an empty capsid, an aggregated AAV particle, a degraded AAV particle, or a combination thereof.
A61. The pharmaceutical composition of embodiment A60, wherein the purified AAV particle comprises a full or a partially-full capsid, and the product-related impurity comprises an empty capsid.
A62. The pharmaceutical composition of embodiment A60, wherein the purified AAV particle comprises a full capsid, and the product-related impurity comprises an empty capsid.
A63. The pharmaceutical composition of embodiment A60, wherein the purified AAV particle consists essentially of a full capsid, and the product-related impurity comprises an empty capsid.
A64. The pharmaceutical composition of embodiment A53, wherein the product-related impurity comprises an aggregated AAV particle, a degraded AAV particle, or a combination thereof.
A65. The pharmaceutical composition of embodiment A64, wherein the purified AAV particle comprises an empty capsid.
A66. The pharmaceutical composition of embodiment A64, wherein the purified AAV particle consists essentially of an empty capsid.
A67. The pharmaceutical composition of embodiment A53, wherein the product-related impurity comprises an aggregated AAV particle or a combination thereof.
A68. The pharmaceutical composition of any one of embodiments A1 to A67, wherein the pharmaceutical composition is in a liquid state.
A69. The pharmaceutical composition of any one of embodiments A1 to A67, wherein the pharmaceutical composition is in a solid or a semi-solid state.
A70. The pharmaceutical composition of any one of embodiments A1 to A69, wherein the pharmaceutical composition maintains or enhances the stability of the purified AAV particle.
A71. The pharmaceutical composition of any one of embodiments A1 to A69, wherein the pharmaceutical composition reduces or prevents aggregation of the purified AAV particle.
A72. The pharmaceutical composition of any one of embodiments A1 to A69, wherein the pharmaceutical composition
   (a) maintains or enhances the stability of the purified AAV particle; and
   (b) reduces or prevents aggregation of the purified AAV particle.
A73. The pharmaceutical composition of embodiment A70 or A72, wherein the stability of the purified AAV particle is maintained or enhanced after one or more freeze/thaw cycles.
A74. The pharmaceutical composition of embodiment A70 or A72, wherein the stability of the purified AAV particle is maintained or enhanced after three or more freeze/thaw cycles.
A75. The pharmaceutical composition of embodiment A71 or A72, wherein the aggregation of the AAV particle is less than 5% after one or more freeze/thaw cycles.
A76. The pharmaceutical composition of embodiment A71 or A72, wherein the aggregation of the AAV particle is less than 2% after one or more freeze/thaw cycles.
A77. The pharmaceutical composition of embodiment A71 or A72, wherein the aggregation of the AAV particle is less than 1% after one or more freeze/thaw cycles.
A78. The pharmaceutical composition of embodiment A71 or A72, wherein the aggregation of the AAV particle is less than 5% after three or more freeze/thaw cycles.
A79. The pharmaceutical composition of embodiment A71 or A72, wherein the aggregation of the AAV particle is less than 2% after three or more freeze/thaw cycles.
A80. The pharmaceutical composition of embodiment A71 or A72, wherein the aggregation of the AAV particle is less than 1% after three or more freeze/thaw cycles.
A81. The pharmaceutical composition of any one of embodiments A73 to A80, wherein the stability and/or aggregation of the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).
A82. The pharmaceutical composition of any one of embodiments A1 to A81, wherein the purified AAV particle is obtained by a method comprising:
   (a) contacting a supernatant comprising AAV particles with a composition comprising a nuclease; and
   (b) purifying the particles.
A83. The pharmaceutical composition of embodiment A82, wherein the nuclease comprises Benzonase, or Benzonase® and a chromatin-DNA nuclease.
A84. The pharmaceutical composition of embodiment A83, wherein the chromatin-DNA nuclease comprises a MNase.
A85. A method for making a pharmaceutical composition comprising a purified AAV particle, the method comprising:
   (a) contacting a supernatant comprising an AAV particle with a composition comprising a nuclease;
   (b) purifying the AAV particle, such that the AAV particle is substantially free of an impurity;
   (c) combining the purified AAV particle with a buffering agent, a cryoprotectant, and a non-ionic surfactant, wherein
      i. the buffering agent concentration is about 0 mM to about 50 mM,
      ii. the cryoprotectant is about 1% to about 10% (w/v), and
      iii. the non-ionic surfactant is about 0.0001% (w/v) to about 0.1% (w/v).
A86. The method of embodiment A85, wherein step (c) further comprises a pharmaceutically acceptable salt, wherein the pharmaceutically acceptable salt concentration is about 1 mM to about 200 mM.
A87. The method of embodiment A86, wherein the pharmaceutically acceptable salt is about 10 mM to about 150 mM.
A88. The method of embodiment A86, wherein the pharmaceutically acceptable salt is about 1 mM to about 49 mM.
A89. The method of embodiment A86, wherein the pharmaceutically acceptable salt is about 5 mM to about 45 mM.
A90. The method of embodiment A86, wherein the pharmaceutically acceptable salt is about 7.5 mM to about 40 mM.
A91. The method of embodiment A86, wherein the pharmaceutically acceptable salt is about 10 mM to about 30 mM.
A92. The method of embodiment A86, wherein the pharmaceutically acceptable salt concentration is about 10 mM.
A93. The method of embodiment A86, wherein the pharmaceutically acceptable salt concentration is about 100 mM.
A94. The method of embodiment A86, wherein the pharmaceutically acceptable salt concentration is about 150 mM.
A95. The method of any one of embodiments A86 to A94, wherein the pharmaceutically acceptable salt is selected from the group consisting of a sodium salt, a magnesium salt, a calcium salt, a potassium salt, a phosphate salt, and a sulfate salt.
A96. The method of embodiment A95, wherein the sodium salt comprises sodium chloride.
A97. The method of any one of embodiments A85 to A96, wherein the buffering agent comprises Tris HCl.
A98. The method of any one of embodiments A85 to A96, wherein the buffering agent comprises L-Histidine HCl.
A99. The method of any one of embodiments A85 to A98, wherein the buffering agent concentration comprises about 20 mM.
A100. The method of any one of embodiments A85 to A99, wherein the cryoprotectant is about 3% (w/v) to about 8% (w/v).
A101. The method of any one of embodiments A85 to A100, wherein the cryoprotectant is about 4% (w/v) to about 6% (w/v).
A102. The method of any one of embodiments A85 to A101, wherein the cryoprotectant is about 5% (w/v).
A103. The method of any one of embodiments A85 to A102, wherein the cryoprotectant comprises a sugar.
A104. The method of embodiment A103, wherein the sugar comprises sucrose, trehalose, or a combination thereof.
A105. The method of embodiment A103, wherein the sugar comprises trehalose.
A106. The method of any one of embodiments A85 to A105, wherein the non-ionic surfactant is about 0.0005% (w/v) to about 0.005% (w/v).
A107. The method of any one of embodiments A85 to A106, wherein the non-ionic surfactant is about 0.00075% (w/v) to about 0.0025% (w/v).
A108. The method of any one of embodiments A85 to A107, wherein the non-ionic surfactant is about 0.001% (w/v).
A109. The method of any one of embodiments A85 to A108, wherein the non-ionic surfactant is selected from the group consisting of a copolymer, a polyoxyethylene sorbitan ester, a phospholipid, a Brij surfactant, and a sorbitan ester, or a combination thereof.
A110. The method of embodiment A109, wherein the polyoxyethylene sorbitan ester is selected from the group consisting of (PS-20), and polysorbate 80 (PS-80), or a combination thereof.
A111. The method of embodiment A109, wherein the copolymer comprises a poloxamer.
A112. The method of embodiment A111, wherein the poloxamer is selected from the group consisting of poloxamer 188 (P188), poloxamer 237 (P237), poloxamer 338 (P338), and poloxamer 407 (P407), or a combination thereof.
A113. The method of embodiment A111, wherein the poloxamer comprises poloxamer 188 (P188).
A114. The method of any one of embodiments A85 to A113, wherein the pharmaceutical composition pH is about 4.0 to about 9.0.
A115. The method of any one of embodiments A85 to A114, wherein the pharmaceutical composition pH is about 7.0 to about 8.0.
A116. The method of any one of embodiments A85 to A115, wherein the pharmaceutical composition pH is about 7.3 to about 7.7.
A117. The method of any one of embodiments A85 to A116, wherein the pharmaceutical composition pH is about 7.5.
A118. The method of any one of embodiments A85 to A117, wherein the AAV is selected from the group consisting of AAV1, AAV2, AAV3, AAV4, AAV5, AAV6, AAV7, AAV8, AAV9, and AAV10.
A119. The method of any one of embodiments A85 to A118, wherein the AAV comprises a recombinant AAV (rAAV).
A120. The method of any one of embodiments A85 to A119, wherein the nuclease comprises Benzonase, or Benzonase® and a chromatin-DNA nuclease.
A121. The method of embodiment A120, wherein the chromatin-DNA nuclease comprises a MNase.
A122. The method of any one of embodiments A85 to A121, wherein the purified AAV particle titer is about 1×10¹⁰ viral genomes per milliliter (vg/mL) or greater.
A123. The method of any one of embodiments A85 to A121, wherein the purified AAV particle titer is about 1×10¹¹ viral genomes per milliliter (vg/mL) or greater.
A124. The method of any one of embodiments A85 to A121, wherein the purified AAV particle titer is about 1×10¹² viral genomes per milliliter (vg/mL) or greater.
A125. The method of any one of embodiments A85 to A121, wherein the purified AAV particle titer is about 1×10¹³ viral genomes per milliliter (vg/mL) or greater.
A126. The method of any one of embodiments A85 to A121, wherein the purified AAV particle titer is about 1×10¹⁴ viral genomes per milliliter (vg/mL) or greater.
A127. The method of any one of embodiments A85 to A121, wherein the purified AAV particle titer is about 1×10¹⁵ viral genomes per milliliter (vg/mL) or greater.
A128. The method of any one of embodiments A85 to A127, wherein the impurity comprises a process-related impurity, a product-related impurity, or a combination thereof.
A129. The method of embodiment A128, wherein the process-related impurity is selected from the group consisting of a residual host-cell component, a residual viral production component, a residual cell culture component, a residual purification component, or a combination thereof.
A130. The method of embodiment A129, wherein the residual host-cell component comprises a host-cell protein, a host-cell DNA, a host-cell RNA, or a combination thereof.
A131. The method of embodiment A130, wherein the host-cell DNA comprises an extra-viral, chromatin-associated DNA.
A132. The method of embodiment A129, wherein the residual viral production component comprises a plasmid DNA, a helper virus, or a combination thereof.
A133. The method of embodiment A129, wherein the residual cell culture component comprises an antibiotic, a supplement, an inducer, a growth factor, or a combination thereof.
A134. The method of embodiment A129, wherein the residual purification component comprises a buffer, an inorganic salt, an enzyme, a detergent, a medium, or a combination thereof
A135. The method of embodiment A128, wherein the product-related impurity comprises an empty capsid, an aggregated AAV particle, a degraded AAV particle, or a combination thereof.
A136. The method of embodiment A135, wherein the purified AAV particle comprises a full or a partially-full capsid, and the product-related impurity comprises an empty capsid.
A137. The method of embodiment A135, wherein the purified AAV particle comprises a full capsid, and the product-related impurity comprises an empty capsid.
A138. The method of embodiment A135, wherein the purified AAV particle consists essentially of a full capsid, and the product-related impurity comprises an empty capsid.
A139. The method of embodiment A128, wherein the product-related impurity comprises an aggregated AAV particle, a degraded AAV particle, or a combination thereof.
A140. The method of embodiment A139, wherein the purified AAV particle comprises an empty capsid.
A141. The method of embodiment A139, wherein the purified AAV particle consists essentially of an empty capsid.
A142. The method of embodiment A128, wherein the product-related impurity comprises an aggregated AAV particle.

In a second set of embodiments, provided are:
B1. A pharmaceutical composition comprising a means for maintaining or enhancing the stability of a purified AAV particle.
B2. The pharmaceutical composition of embodiment B1, wherein the stability of the purified AAV particle is maintained or enhanced after one or more freeze/thaw cycles.
B3. The pharmaceutical composition of embodiment B1, wherein the stability of the purified AAV particle is maintained or enhanced after three or more freeze/thaw cycles.
B4. The pharmaceutical composition of any one of embodiments B1 to B3, wherein the stability of the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).
B5. A pharmaceutical composition comprising a means for decreasing or preventing aggregation of a purified AAV particle.
B6. The pharmaceutical composition of embodiment B5, wherein the aggregation of the AAV particle is less than 5% after one or more freeze/thaw cycles.
B7. The pharmaceutical composition of embodiment B5, wherein the aggregation of the AAV particle is less than 2% after one or more freeze/thaw cycles.
B8. The pharmaceutical composition of embodiment B5, wherein the aggregation of the AAV particle is less than 1% after one or more freeze/thaw cycles.
B9. The pharmaceutical composition of embodiment B5, wherein the aggregation of the AAV particle is less than 5% after three or more freeze/thaw cycles.
B10. The pharmaceutical composition of embodiment B5, wherein the aggregation of the AAV particle is less than 2% after three or more freeze/thaw cycles.
B11. The pharmaceutical composition of embodiment B5, wherein the aggregation of the AAV particle is less than 1% after three or more freeze/thaw cycles.
B12. The pharmaceutical composition of any one of embodiments B5 to B11, wherein the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).
B13. A pharmaceutical composition comprising a means for (a) for maintaining or enhancing the stability of a purified AAV particle, and (b) decreasing or preventing aggregation of a purified AAV particle.
B14. The pharmaceutical composition of embodiment B13, wherein the stability of the purified AAV particle is maintained or enhanced after one or more freeze/thaw cycles.
B15. The pharmaceutical composition of embodiment B13, wherein the stability of the purified AAV particle is maintained or enhanced after three or more freeze/thaw cycles.
B16. The pharmaceutical composition of embodiment B13, wherein the aggregation of the AAV particle is less than 5% after one or more freeze/thaw cycles.
B17. The pharmaceutical composition of embodiment B13, wherein the aggregation of the AAV particle is less than 2% after one or more freeze/thaw cycles.
B18. The pharmaceutical composition of embodiment B13, wherein the aggregation of the AAV particle is less than 1% after one or more freeze/thaw cycles.
B19. The pharmaceutical composition of embodiment B13, wherein the aggregation of the AAV particle is less than 5% after three or more freeze/thaw cycles.
B20. The pharmaceutical composition of embodiment B13, wherein the aggregation of the AAV particle is less than 2% after three or more freeze/thaw cycles.
B21. The pharmaceutical composition of embodiment B13, wherein the aggregation of the AAV particle is less than 1% after three or more freeze/thaw cycles.
B22. The pharmaceutical composition of any one of embodiments B13 to B21, wherein the stability and aggregation of the AAV particle are measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).
B23. A system comprising a means for making and obtaining a pharmaceutical composition comprising a purified adeno-associated virus (AAV) particle, a buffering agent, a cryoprotectant, and a non-ionic surfactant, wherein
   (a) the purified AAV particle is substantially free of an impurity,
   (b) the buffering agent concentration is about 0 mM to about 50 mM,
   (c) the cryoprotectant is about 1% to about 10% (w/v), and
   (d) the non-ionic surfactant is about 0.0001% (w/v) to about 0.1% (w/v).
B24. The system of embodiment B23, wherein the pharmaceutical composition further comprises a pharmaceutically acceptable salt, wherein the pharmaceutically acceptable salt concentration is about 1 mM to about 200 mM.
B25. The system of embodiment B24, wherein the pharmaceutically acceptable salt is about 10 mM to about 150 mM.
B26. The system of embodiment B24 or 25, wherein the pharmaceutically acceptable salt concentration is about 10 mM.
B27. The system of embodiment B24 or 25, wherein the pharmaceutically acceptable salt concentration is about 100 mM.
B28. The system of embodiment B24 or 25, wherein the pharmaceutically acceptable salt concentration is about 150 mM.
B29. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   a. the purified AAV particle is substantially free of an impurity,
   b. the pharmaceutically acceptable salt concentration is about 1 mM to about 49 mM,
   c. the buffering agent concentration is about 0 mM to about 50 mM,
   d. the cryoprotectant is about 1% to about 10% (w/v), and
   e. the non-ionic surfactant is about 0.0001% (w/v) to about 0.1% (w/v).
B30. The system of embodiment B29, wherein the pharmaceutically acceptable salt concentration is about 5 mM to about 45 mM.
B31. The system of embodiment B29 or B30, wherein the pharmaceutically acceptable salt concentration is about 7.5 mM to about 40 mM.
B32. The system of any one of embodiments B29 to B31, wherein the pharmaceutically acceptable salt concentration is about 10 mM to about 30 mM.
B33. The system of any one of embodiments B29 to B32, wherein the pharmaceutically acceptable salt concentration is about 10 mM.
B34. The system of any one of embodiments B24 to B33, wherein the pharmaceutically acceptable salt is selected from the group consisting of a sodium salt, a magnesium salt, a calcium salt, a potassium salt, a phosphate salt, and a sulfate salt.
B35. The system of embodiment B34, wherein the sodium salt comprises sodium chloride.
B36. The system of any one of embodiments B23 to B35, wherein the buffering agent comprises Tris HCl.
B37. The system of any one of embodiments B23 to B35, wherein the buffering agent comprises L-Histidine HCl.
B38. The system of any one of embodiments B23 to B37, wherein the buffering agent concentration comprises about 20 mM.
B39. The system of any one of embodiments B23 to B38, wherein the cryoprotectant is about 3% (w/v) to about 8% (w/v).
B40. The system of any one of embodiments B23 to B39, wherein the cryoprotectant is about 4% (w/v) to about 6% (w/v).
B41. The system of any one of embodiments B23 to B40, wherein the cryoprotectant is about 5% (w/v).
B42. The system of any one of embodiments B23 to B41, wherein the cryoprotectant comprises a sugar.
B43. The system of embodiment B42, wherein the sugar comprises sucrose, trehalose, or a combination thereof
B44. The system of embodiment B42, wherein the sugar comprises trehalose.
B45. The system of any one of embodiments B23 to B44, wherein the non-ionic surfactant is about 0.0005% (w/v) to about 0.005% (w/v).
B46. The system of any one of embodiments B23 to B45, wherein the non-ionic surfactant is about 0.00075% (w/v) to about 0.0025% (w/v).
B47. The system of any one of embodiments B23 to B46, wherein the non-ionic surfactant is about 0.001% (w/v).
B48. The system of any one of embodiments B23 to B47, wherein the non-ionic surfactant is selected from the group consisting of a copolymer, a polyoxyethylene sorbitan ester, a phospholipid, a Brij surfactant, and a sorbitan ester, or a combination thereof
B49. The system of embodiment B48, wherein the polyoxyethylene sorbitan ester is selected from the group consisting of (PS-20), and polysorbate 80 (PS-80), or a combination thereof.
B50. The system of embodiment B48, wherein the copolymer comprises a poloxamer.
B51. The system of embodiment B50, wherein the poloxamer is selected from the group consisting of poloxamer 188 (P188), poloxamer 237 (P237), poloxamer 338 (P338), and poloxamer 407 (P407), or a combination thereof.
B52. The system of embodiment B50, wherein the poloxamer comprises poloxamer 188 (P188).
B53. A system comprising a means for making and obtaining a pharmaceutical composition comprising a purified adeno-associated virus (AAV) particle, a buffering agent, a cryoprotectant, and a non-ionic surfactant, wherein
   (a) the purified AAV particle is substantially free of an impurity,
   (b) the buffering agent concentration is about 20 mM,
   (c) the cryoprotectant is about 5% (w/v) trehalose, and
   (d) the non-ionic surfactant is about 0.001% (w/v) poloxamer 188.
B54. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 10 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B55. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 25 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B56. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 50 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B57. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 100 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B58. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 125 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B59. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 150 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B60. A system comprising a means for making and obtaining a pharmaceutical composition comprising a purified adeno-associated virus (AAV) particle, a pharmaceutically acceptable salt, a buffering agent, a cryoprotectant, and a non-ionic surfactant, wherein
   (a) the purified AAV particle is substantially free of an impurity,
   (b) the pharmaceutically acceptable salt concentration is about 200 mM sodium chloride,
   (c) the buffering agent concentration is about 20 mM,
   (d) the cryoprotectant is about 5% (w/v) trehalose, and
   (e) the non-ionic surfactant is about 0.001% (w/v) poloxamer 188.
B61. The system of any one of embodiments B53 to B60 wherein the buffering agent comprises Tris HCl.
B62. The system of any one of embodiments B53 to B60, wherein the buffering agent comprises L-Histidine HCl.
B63. The system of any one of embodiments B23 to B62, wherein the pharmaceutical composition pH is about 4.0 to about 9.0.
B64. The system of any one of embodiments B23 to B63, wherein the pharmaceutical composition pH is about 7.0 to about 8.0.
B65. The system of any one of embodiments B23 to B64, wherein the pharmaceutical composition pH is about 7.3 to about 7.7.
B66. The system of any one of embodiments B23 to B65, wherein the pharmaceutical composition pH is about 7.5.
B67. The system of any one of embodiments B23 to B66, wherein the AAV is selected from the group consisting of AAV1, AAV2, AAV3, AAV4, AAV5, AAV6, AAV7, AAV8, AAV9, and AAV10.
B68. The system of any one of embodiments B23 to B67, wherein the AAV comprises a recombinant AAV (rAAV).
B69. The system of any one of embodiments B23 to B68, wherein the purified AAV particle titer is about 1×10¹⁰ viral genomes per milliliter (vg/mL) or greater.
B70. The system of any one of embodiments B23 to B68, wherein the purified AAV particle titer is about 1×10¹¹ viral genomes per milliliter (vg/mL) or greater.
B71. The system of any one of embodiments B23 to B68, wherein the purified AAV particle titer is about 1×10¹² viral genomes per milliliter (vg/mL) or greater.
B72. The system of any one of embodiments B23 to B68, wherein the purified AAV particle titer is about 1×10¹³ viral genomes per milliliter (vg/mL) or greater.
B73. The system of any one of embodiments B23 to B68, wherein the purified AAV particle titer is about 1×10¹⁴ viral genomes per milliliter (vg/mL) or greater.
B74. The system of any one of embodiments B23 to B68, wherein the purified AAV particle titer is about 1×10¹⁵ viral genomes per milliliter (vg/mL) or greater.
B75. The system of any one of embodiments B23 to B74, wherein the impurity comprises a process-related impurity, a product-related impurity, or a combination thereof.
B76. The system of embodiment B75, wherein the process-related impurity is selected from the group consisting of a residual host-cell component, a residual viral production component, a residual cell culture component, a residual purification component, or a combination thereof.
B77. The system of embodiment B76, wherein the residual host-cell component comprises a host-cell protein, a host-cell DNA, a host-cell RNA, or a combination thereof.
B78. The system of embodiment B77, wherein the host-cell DNA comprises an extra-viral, chromatin-associated DNA.
B79. The system of embodiment B78, wherein the residual viral production component comprises a plasmid DNA, a helper virus, or a combination thereof.
B80. The system of embodiment B76, wherein the residual cell culture component comprises an antibiotic, a supplement, an inducer, a growth factor, or a combination thereof.
B81. The system of embodiment B76, wherein the residual purification component comprises a buffer, an inorganic salt, an enzyme, a detergent, a medium, or a combination thereof.
B82. The system of embodiment B75, wherein the product-related impurity comprises an empty capsid, an aggregated AAV particle, a degraded AAV particle, or a combination thereof
B83. The system of embodiment B82, wherein the purified AAV particle comprises a full or a partially-full capsid, and the product-related impurity comprises an empty capsid.
B84. The system of embodiment B82, wherein the purified AAV particle comprises a full capsid, and the product-related impurity comprises an empty capsid.
B85. The system of embodiment B82, wherein the purified AAV particle consists essentially of a full capsid, and the product-related impurity comprises an empty capsid.
B86. The system of embodiment B75, wherein the product-related impurity comprises an aggregated AAV particle, a degraded AAV particle, or a combination thereof.
B87. The system of embodiment B86, wherein the purified AAV particle comprises an empty capsid.
B88. The system of embodiment B86, wherein the purified AAV particle consists essentially of an empty capsid.
B89. The system of embodiment B75, wherein the product-related impurity comprises an aggregated AAV particle or a combination thereof.
B90. The system of any one of embodiments B23 to B89, wherein the pharmaceutical composition is in a liquid state.
B91. The system of any one of embodiments B23 to B89, wherein the pharmaceutical composition is in a solid or a semi-solid state.
B92. The system of any one of embodiments B23 to B91, wherein the pharmaceutical composition maintains or enhances the stability of the purified AAV particle.
B93. The system of any one of embodiments B23 to B91, wherein the pharmaceutical composition reduces or prevents aggregation of the purified AAV particle.
B94. The system of any one of embodiments B23 to B91, wherein the pharmaceutical composition
   (a) maintains or enhances the stability of the purified AAV particle; and
   (b) reduces or prevents aggregation of the purified AAV particle.
B95. The system of embodiment B92 or B94, wherein the stability of the purified AAV particle is maintained or enhanced after one or more freeze/thaw cycles.
B96. The system of embodiment B92 or B94, wherein the stability of the purified AAV particle is maintained or enhanced after three or more freeze/thaw cycles.
B97. The system of embodiment B93 or B94, wherein the aggregation of the AAV particle is less than 5% after one or more freeze/thaw cycles.
B98. The system of embodiment B93 or B94, wherein the aggregation of the AAV particle is less than 2% after one or more freeze/thaw cycles.
B99. The system of embodiment B93 or B94, wherein the aggregation of the AAV particle is less than 1% after one or more freeze/thaw cycles.
B100. The system of embodiment B93 or B94, wherein the aggregation of the AAV particle is less than 5% after three or more freeze/thaw cycles.
B101. The system of embodiment B93 or B94, wherein the aggregation of the AAV particle is less than 2% after three or more freeze/thaw cycles.
B102. The system of embodiment B93 or B94, wherein the aggregation of the AAV particle is less than 1% after three or more freeze/thaw cycles.
B103. The system of any one of embodiments B93 to B102, wherein the stability and/or aggregation of the AAV particle is measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).
B104. The system of any one of embodiments B23 to B103, wherein the purified AAV particle is obtained by a method comprising:
   (a) contacting a supernatant comprising AAV particles with a composition comprising a nuclease; and
   (b) purifying the particles.
B105. The system of embodiment B104, wherein the nuclease comprises Benzonase, or Benzonase® and a chromatin-DNA nuclease.
B106. The system of embodiment B105, wherein the chromatin-DNA nuclease comprises a MNase.

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

### 7. EXAMPLES

The following examples of the application are to further illustrate the nature of the application. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description. AAV2/8 particles were used in the following examples as a representative AAV species, however, it is understood that the formulations are suitable for any AAV particle.

### Example I: Purified AAV Particles in Low Ionic Strength Formulations Exhibited Physical Stability after Freeze/Thaws

This example establishes that purified AAV particles in formulations with salt concentrations of 200 mM or less exhibited physical stability after free/thaws.

To determine whether formulations with a low salt concentration could maintain the physical stability of purified AAV particles after one or more freeze/thaws, purified AAV2/8 particles were used a representative AAV particle species and formulated in one of the formulations described in Table 1.

TABLE 1
Formulation compositions

| | Formulation | Formulation | Formulation | Formulation |
|---|---|---|---|---|
| | #1 | #2 | #3 | #4 |
| Concentra- | >1E13 | >1E13 | >1E13 | >1E13 |
| tion | | | | |
| (vg/mL) | | | | |
| Buffer | 20 mM | 20 mM | 20 mM | 20 mM |
| (mM), | Tris-HCl | Tris-HCl | Tris-HCl | Tris-HCl |
| pH | pH 7.5 | pH 7.5 | pH 7.5 | pH 7.5 |
| Salt (mM) | 10 mM | 100 mM | 150 mM | 200 mM |
| | NaCl | NaCl | NaCl | NaCl |
| Cryo- | 5% | 5% | 5% | 5% |
| protectant | Trehalose | Trehalose | Trehalose | Trehalose |
| (% w/v) | | | | |
| Surfactant | 0.001% | 0.001% | 0.001% | 0.001% |
| (% w/v) | P188 | P188 | P188 | P188 |

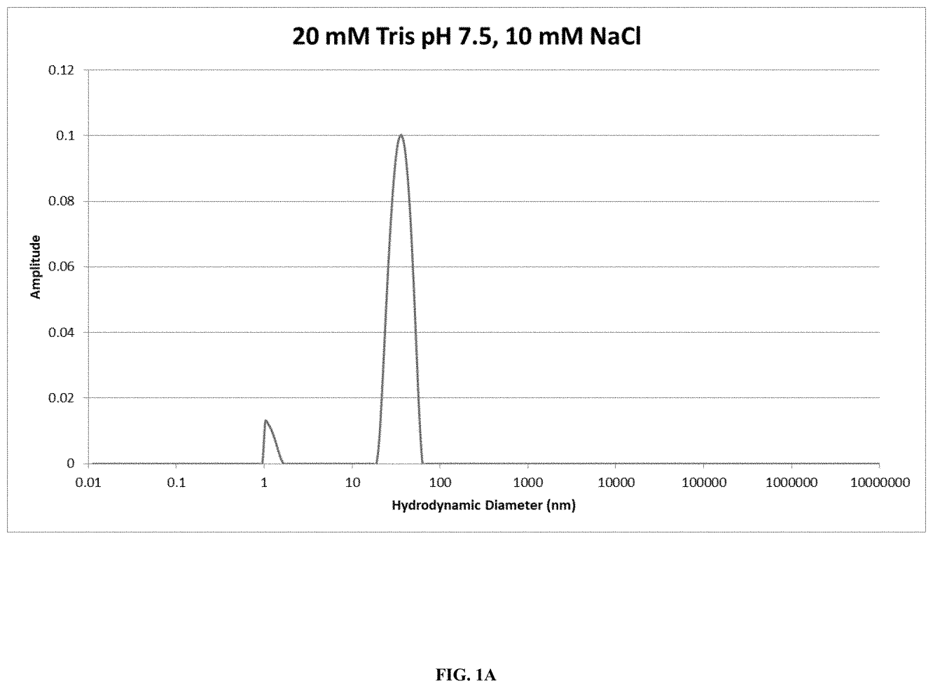

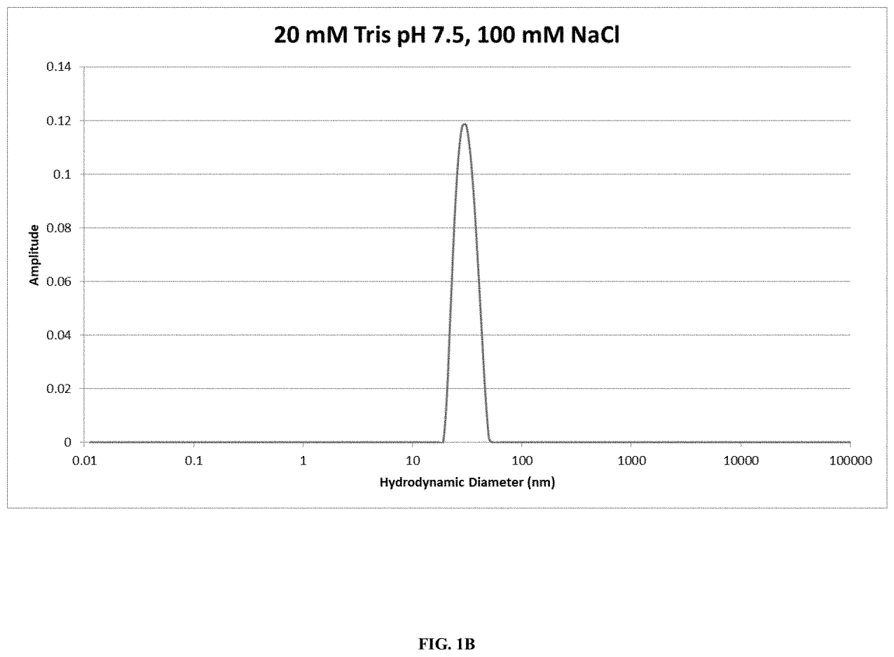

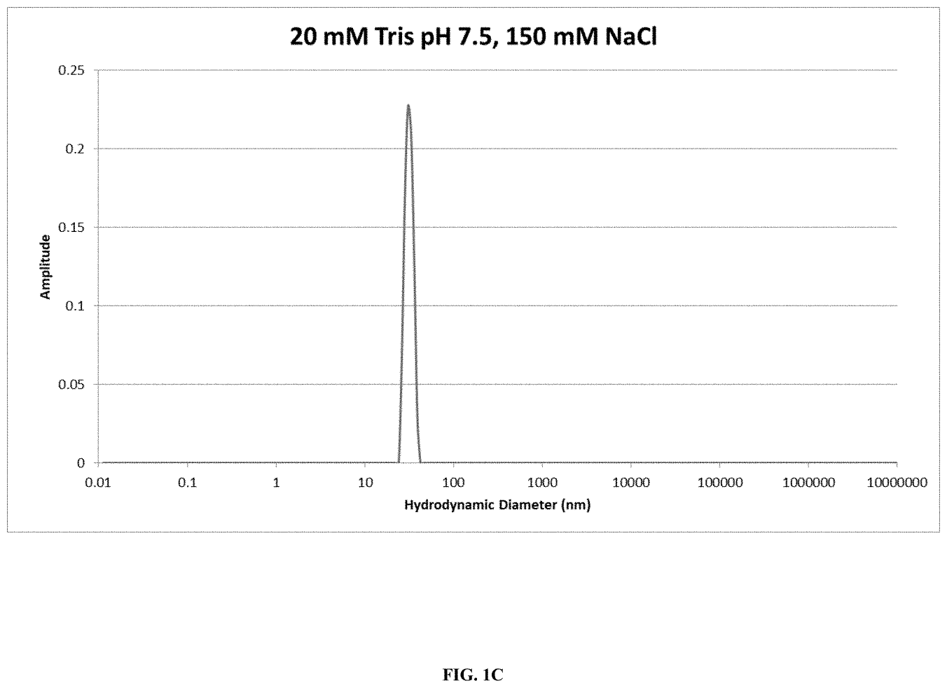

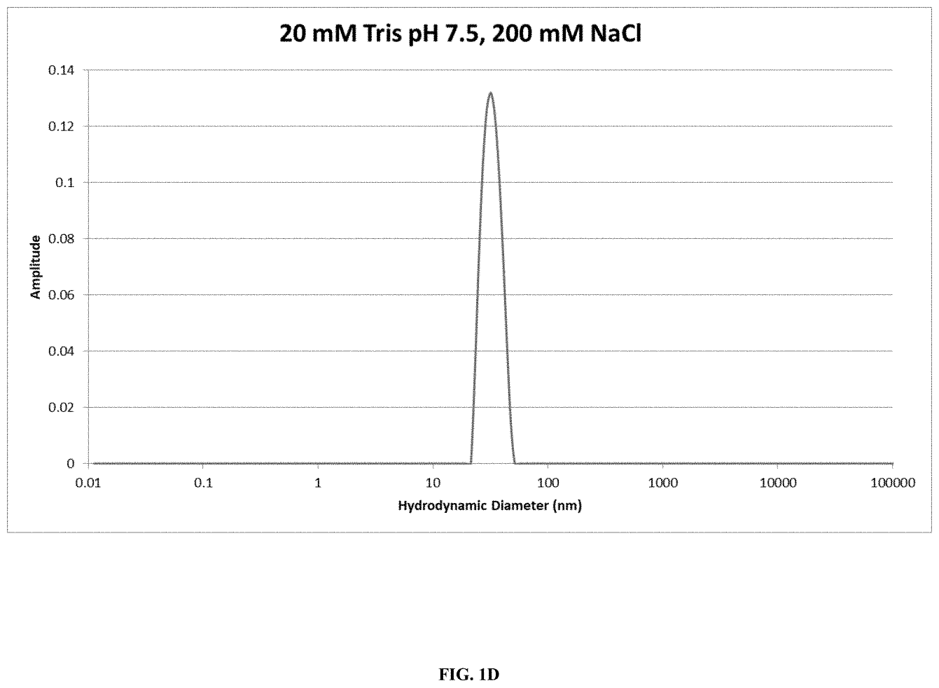

Briefly, following production and purification, AAV particles were formulated in the Formulation #1, Formulation #2, Formulation #3, or Formulation #4. The physical properties of the AAV particles were then characterized following formulation using dynamic light scatter (DLS). Representative DLS data for the four formulations is shown in FIG. 1A-FIG. 1D. Measurement of the hydrodynamic diameter of the AAV particles for each of the four formulations revealed that all four formulations resulted in a homogeneous particle size following the initial formulation, with little to no aggregation.

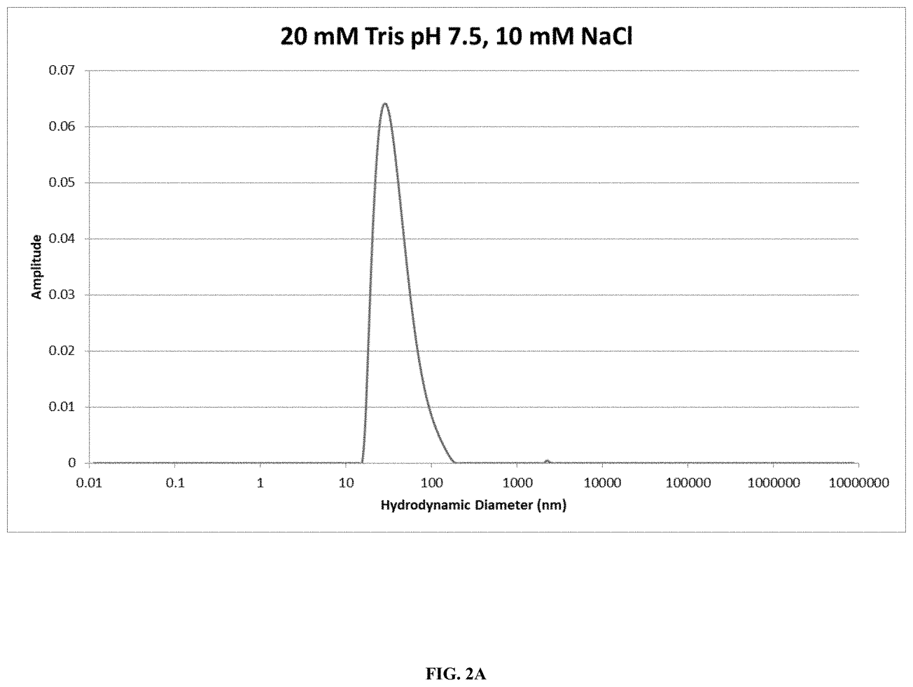

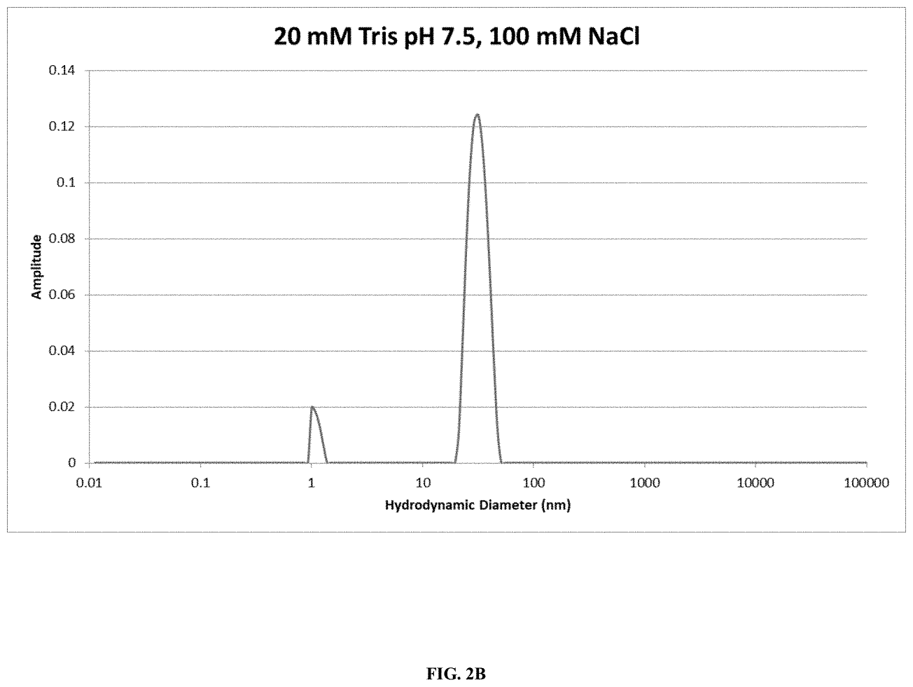

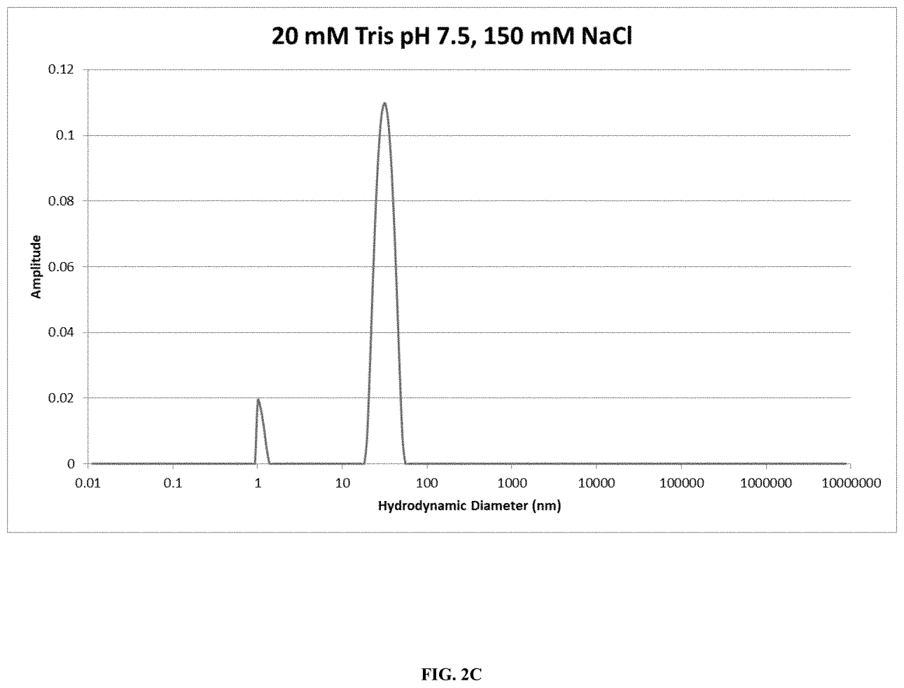

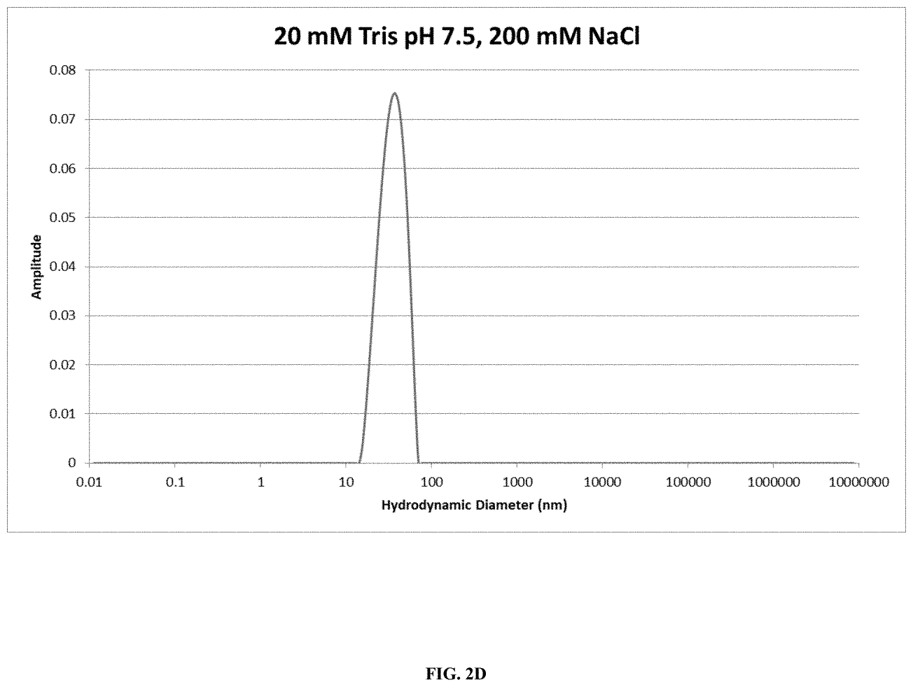

Next, the formulations were frozen and subjected to a single freeze/thaw cycling for evaluation of physical stability (FIG. 2A-FIG. 2D). Particle size data was measured by DLS for the four representative formulations after a single freeze/thaw cycle and robust physical stability was observed across all formulations, relative to pre-freeze DLS. Specifically, AAV particles formulated in the Formulation #1, Formulation #2, Formulation #3, or Formulation #4 had a monodispersed peaks, indicating little to no aggregation. The peak at 1 nm is a common excipient peak, but is not associated with aggregation of AAVs.

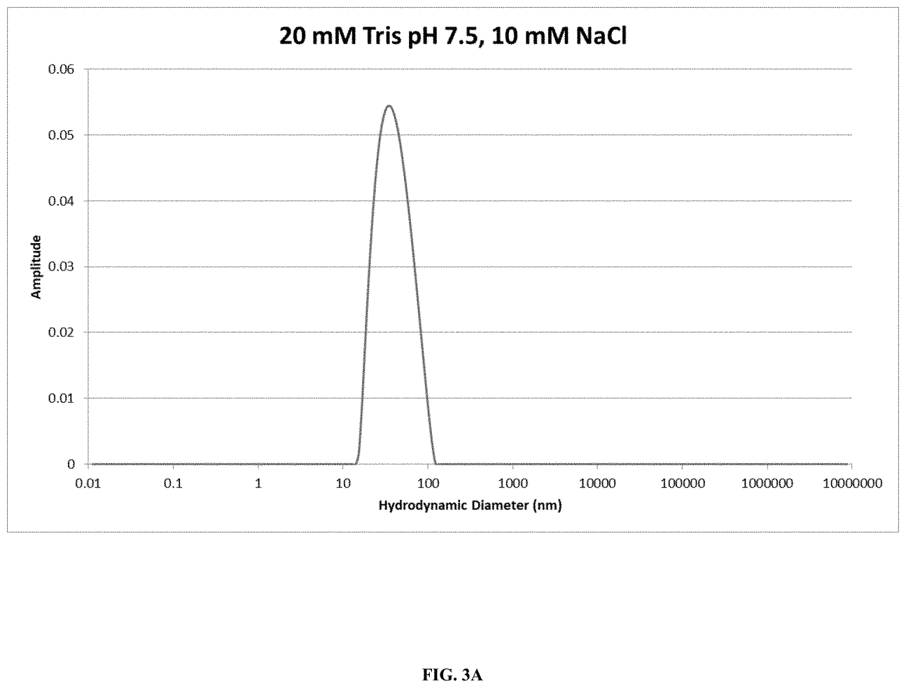

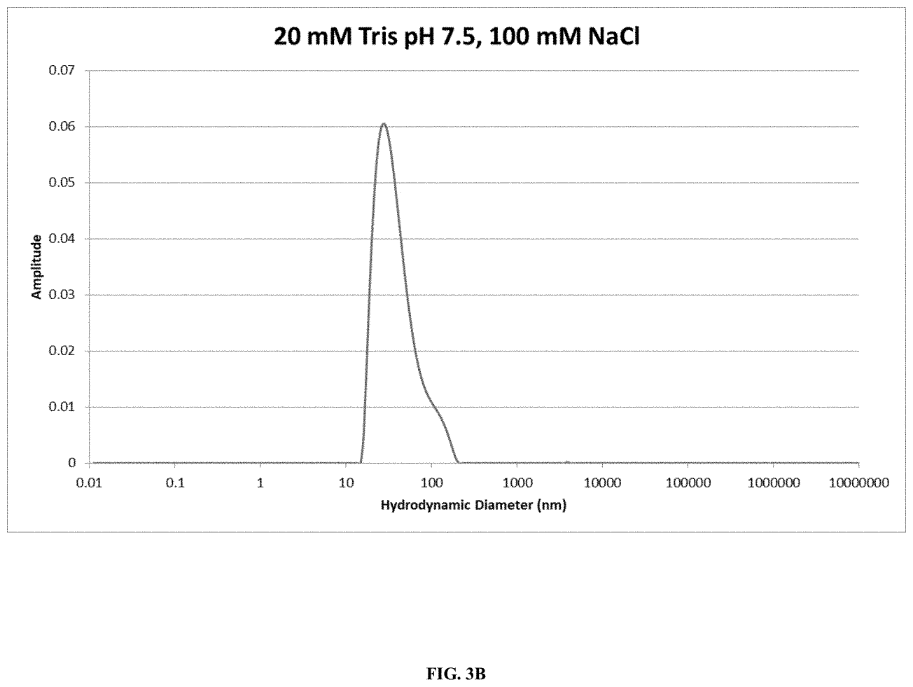

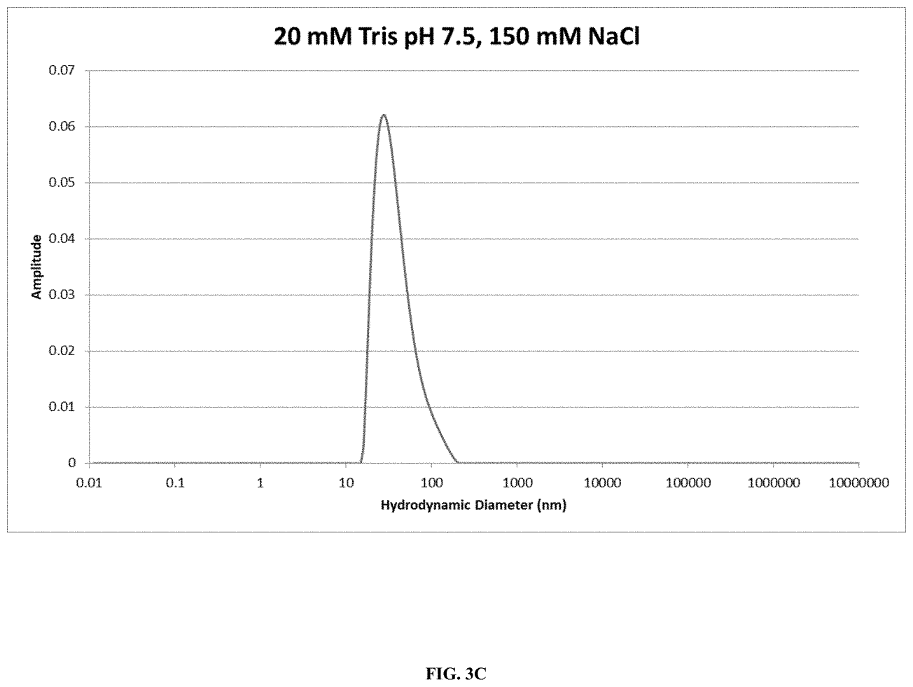

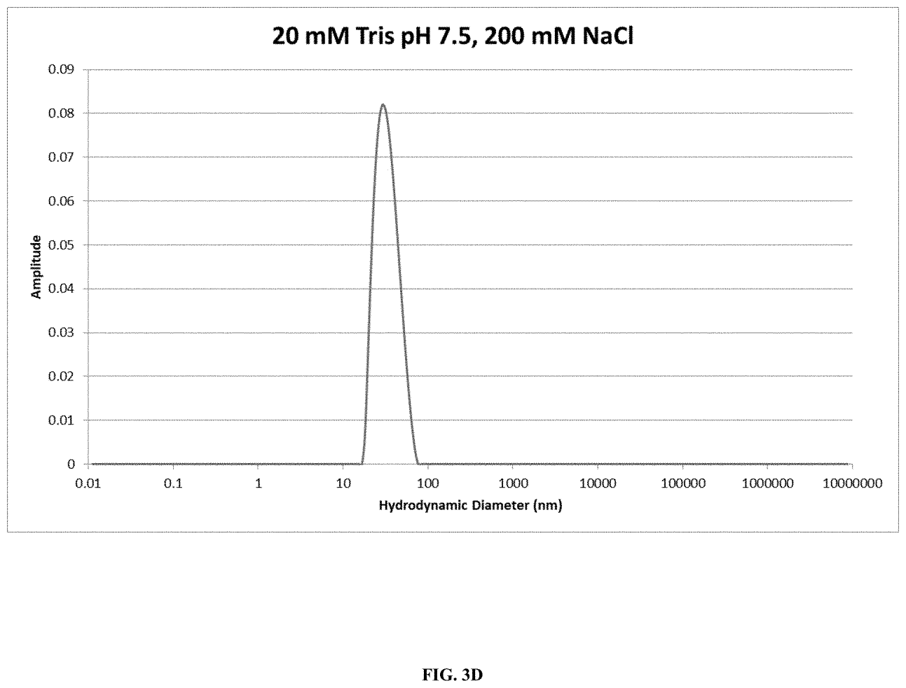

Similar results were observed following three freeze/thaw cycles (FIG. 3A-FIG. 3D). Specifically, the DLS results following three freeze/thaw cycles depicted monodisperse peaks with very minor tailing in Formulation #2 and Formulation #3.

Taken together, this example demonstrates that AAV particles in formulations with low ionic strength (e.g., salt≤200 mM) exhibited robust physical stability, even after multiple freeze/thaw cycles.

### Example II: Detection of Impurities and Empty Capsids in AAV Particles

This example establishes that chromatography and UV absorbance spectra at 280 nm (protein) and 260 nm (DNA/RNA) in the product and post-product fractions can be used to detect impurities in purified AAV particles, as well as distinguish between full and empty capsids. In addition, this example illustrates using an exemplary method involving MNase treatment that the amount of impurities and empty capsids (where undesirable) can be substantially reduced from the purified AAV particles.

Impurities in AAV particles, such as extra-viral, chromatin-associated AAV particles, are an undesirable product. These impurities can cause visible precipitation of purified product, which can be problematic for any formulation studies. Moreover, increased chromatin/DNA binding protein are undesirable contaminants, both of which can increase host-cell protein/DNA contamination. Further, in certain settings empty capsids may also be undesirable. The present studies demonstrated that the chromatin-DNA nuclease, MNase, can be added during purification and results in an improved purification of AAV particles.

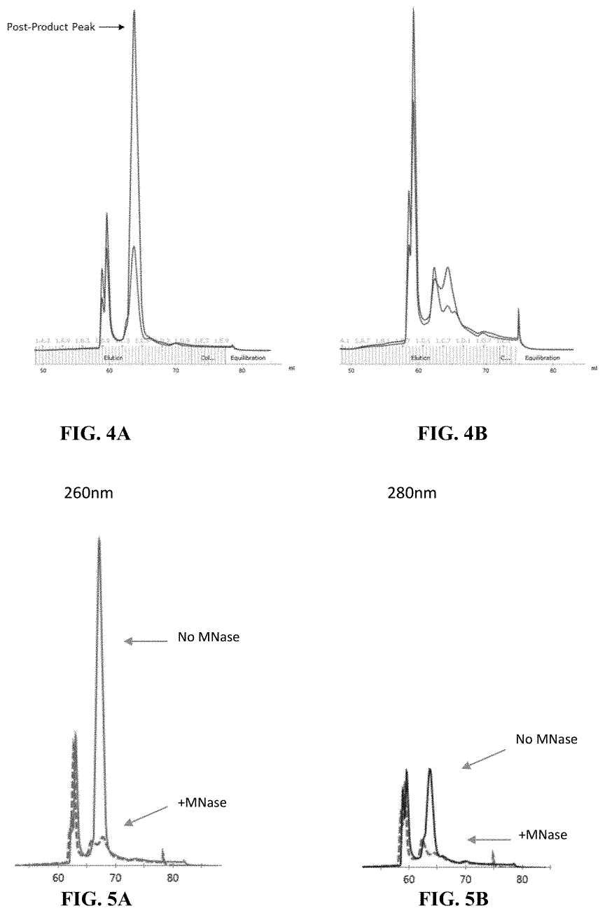

Recombinant AAV2/8 particles were produced as an exemplary AAV particle species. The AAV particles were produced using an exemplary method that involved suspension culture and purification using an anion exchange chromatography over a Bia Separations CIM Disk (0.34 mL column volume, 1.3 um pore size) with or without MNase treatment. DNA (absorbance at 260 nm) and protein (absorbance at 280 nm) was measured in the product and post-product fractions. The results indicated that in the samples purified without MNase treatment, there was large amounts of DNA and protein in the post-product fraction (FIG. 4A), which was substantially reduced by MNase treatment (FIG. 4B). Specifically, 60 U/mL of MNase treatment reduced both the large 260 nm (DNA/RNA) absorbance peak, and the 280 nm (protein) absorbance peak of the post-product peak (FIG. 4B).

A direct comparison of the post-product peak heights for DNA/RNA (260 nm) (FIG. 5A) and protein (280 nm) (FIG. 5B) by overlaying the affinity exchange chromatogram of rAAV8 particles produced from suspension culture with (dashed lines) or without MNase (solid lines) treatment provided further evidence that the exemplary method for AAV particle purification involving MNase treatment for chromatin digestion enhances purification of AAV particles, and significantly reduced, to undetectable levels, residual host-cell protein contamination (FIG. 5A and FIG. 5B, respectively). In particular, the reduction observed with the nucleic acid spectrum (260 nm), post-product peak height was quite significant, estimated to be over 90% reduction in peak height and area (FIG. 5A). In addition, a reduction in the protein spectrum (280 nm) post-product peak was also observed (FIG. 5B).

Accordingly, the results described above evidenced that impurities in the purified AAV particles can be detected by using chromatography and measuring UV absorbance spectra at 280 nm (protein) and 260 nm (DNA/RNA) in the product and post-product fractions. Moreover, the amount of impurities in the purified AAV particles can be substantially reduced using purification techniques, such as treating the AAV particles with MNase.

### Example III: Reduction of Product-Related Impurities in AAV Particles

This example establishes that product-related impurities (e.g., aggregated AAV particles) can be measured in AAV particles by microscopy, and that the exemplary method involving MNase treatment can reduce the product-related impurities.

To demonstrate that purified AAV particles can be substantially free of product-related impurities, such as aggregated AAV particles and precipitated AAV particles, macroscopic and microscopic images were obtained from samples using different purification techniques. The exemplary techniques involved purification of AAV particles with or without MNase treatment. The results evidenced that AAV particles could be generated to be substantially free of visible participates.

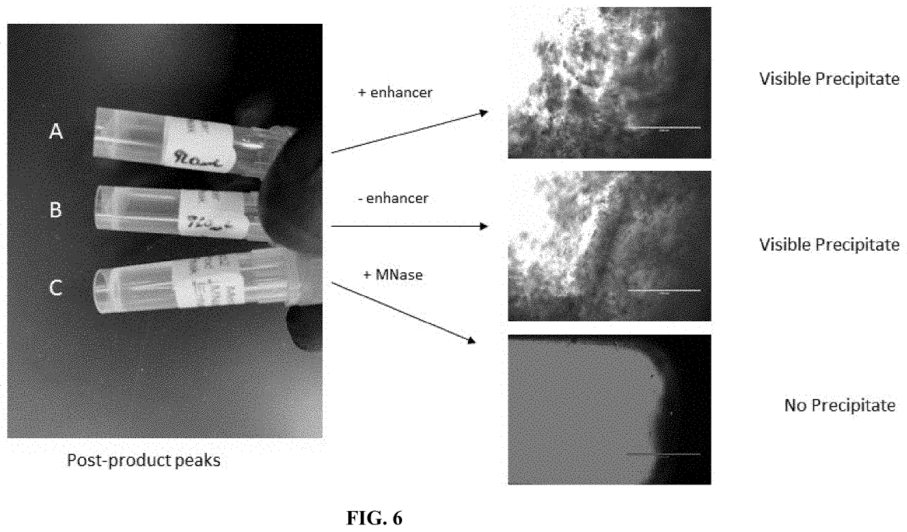

For example, macroscopic examination of the post-product peak fractions showed that non-MNase treated rAAV8 particles produced in suspension Expi293F™ cells (Thermo Fisher Scientific) using the ExpiFectamine™ 293 Transfection Kit Enhancer (Thermo Fisher Scientific), or non-MNase treated rAAV8 particles produced in suspension Expi293F™ cells without Enhancer had a visible precipitate under macroscopic examination (FIG. 6). In contrast, rAAV8 particles produced in suspension Expi293F™ cells without Enhancer and digested with 60 U/mL MNase at 25° C. for 30 minutes had no visible precipitate (FIG. 6).

This data demonstrated that AAV particles purified such that they are substantially free of product-related impurities, aggregated AAV particles and precipitated AAV particles. Further, the results exemplified that AAV particle impurities can be visualized by microscopy.

### Example IV: Generation of High Titers of Purified AAV Particles

This example establishes that high titers of purified AAV particles can be generated and measured.

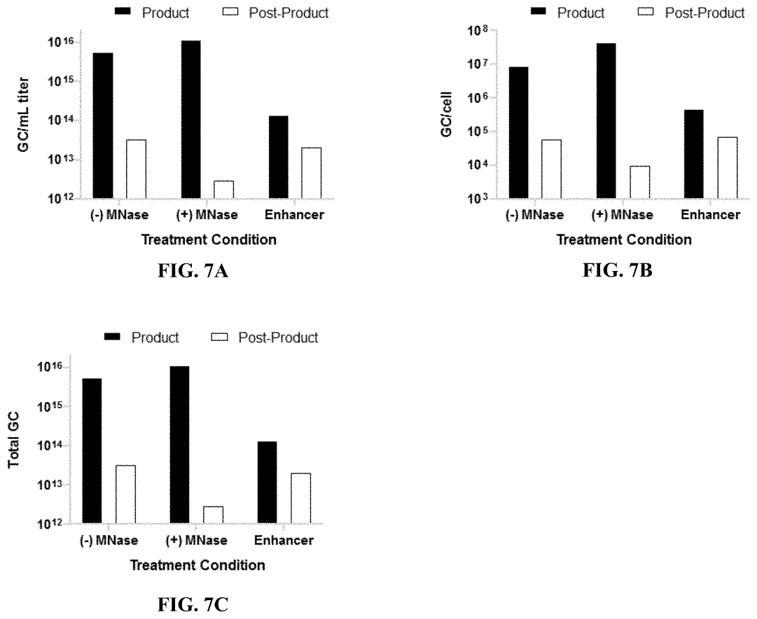

AAV particles were purified using three exemplary purification techniques, and the viral titer was measured. Briefly, AAV2/8 particles were prepared as exemplary AAV particles, and purified using the exemplary purification techniques that included either (1) no MNase; (2) MNase; or (3) an enhancer. Quantification of the genome copies per mL (GC/mL) titers (FIG. 7A), genome copies per cell (GC/cell) (FIG. 7B), and total genome copies (FIG. 7C) revealed that high titers of purified AAV particles could be generated.

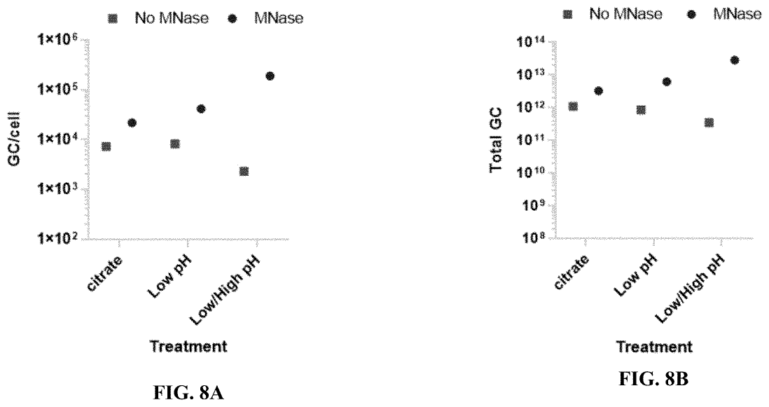

In addition, high titers of purified AAV vectors were also produced from samples treated with or without MNase, and eluted using (1) high/low pH elution; (2) citrate elution, or (3) low pH elution. As shown in FIG. 8B, the total genome copies ranged from 1×10¹¹-1×10¹⁴ (FIG. 8B, Table 2) was consistently observed in MNase treated samples for each of the three different elution buffers (i.e., citrate, low pH, and low/high pH).

TABLE 2
Total Genome Copies

| | No MNase | MNase |
|---|---|---|
| Citrate | 1.09e¹² | 3.27e¹² |
| Low pH | 8.48e¹¹ | 6.22e¹² |
| Low/High pH | 3.45e¹¹ | 2.84e¹³ |

Taken together, these data demonstrate that AAV particles can be purified to generate high viral titers, and the viral titers can be measured.

### Example V: High pH Wash Improved Purity of AAV Particles

This example establishes that a high pH wash during affinity exchange chromatography of AAV particles was able to reduce the presence of impurities.

To determine exemplary conditions for purifying AAV particles that could remove impurities, the AKTA system was used and samples were subjected to various wash and/or enzymatic treatment conditions during purification. The different purification conditions are summarized in Table 3.

TABLE 3
Purification Conditions

| | Affinity | | | | |
|---|---|---|---|---|---|
| Condi- | Wash | Benzo- | | | Anion Exchange |
| tion | pH | nase | MNase | Elution | Elution |
| 1 | n/a | No | No | Citrate, | 20 mM BTP, pH 10.2; |
| | | | | pH 2.5 | 10 mM - 200 mM NaCl |
| 2 | n/a | Yes | No | Citrate, | Linear Gradient |
| | | | | pH 2.5 | |
| 3 | n/a | Yes | Yes | H₃PO₄, | |
| | | | | pH 1.5 | |
| 4 | pH 9.5 | Yes | Yes | Citrate, | |
| | | | | pH 2.5 | |
| 5 | pH 10.2 | Yes | Yes | H₃PO₄, | |
| | | | | pH 1.5 | |
| 6 | pH 10.3 | Yes | Yes | H₃PO₄, | |
| | | | | pH 1.5 | |
| 7 | pH 10.3 | Yes | No | H₃PO₄, | |
| | | | | pH 1.5 | |
| 8 | pH 10.9 | Yes | Yes | H₃PO₄, | |
| | | | | pH 1.5 | |
| 9 | pH 10.4 | Yes | Yes | H₃PO₄, | |
| | | | | pH 1.5 | |
| 10 | pH 10.4 | Yes | Yes | H₃PO₄, | 20 mM Tris pH 9.0; |
| | | | | pH 1.5 | 0-300 mM (C₂H₅)NCl |
| | | | | | Linear Gradient |

BTP: Bis-Tris-Propane;
NaCl: sodium chloride;
H3PO4: phosphoric acid

Briefly, after bulk harvest and benzonase treatment, the crude samples were subjected to affinity chromatography. As indicated in Table 3, under specific conditions (i.e., conditions 4-10), the affinity column was washed with a high pH buffer that ranged from pH 9.5 to pH 10.9. In addition, as indicated in Table 3, the samples were either not treated with benzonase or MNase (condition 1), treated on-column with benzonase only (conditions 2, and 7) or treated on-column with benzonase and MNase (conditions 3-5, and 8-10). Elution from the affinity column was performed using citrate, pH 2.5 (conditions 1, 2, and 4), or phosphoric acid (H₃PO₄), pH 1.5 (conditions 3, and 5-10).

After the affinity chromatography purification, the samples were then polished by anion-exchange chromatography using CIM QA (quaternary amine) monolith columns. Thereafter, the samples were either eluted using a 10 mM-200 mM NaCl linear gradient and neutralized in 20 mM BTP, pH 10.2 (conditions 1-9) or eluted using a 0-300 mM (C₂H₅)NCl linear gradient and neutralized in 20 mM Tris pH 9.0 (condition 10).

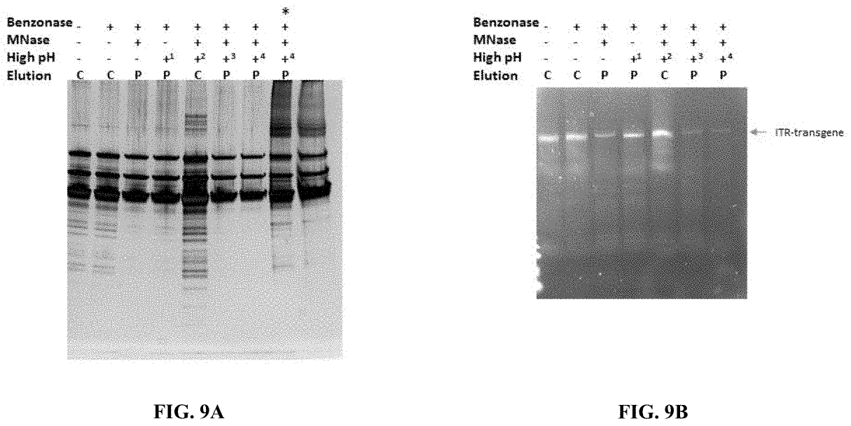

The purified samples were collected and subjected to sodium dodecyl sulfate-polyacrylamide gel electrophoresis (SDS-PAGE) followed by silver staining to detect whether impurities were present in the purified AAV particles. Comparison of the different exemplary purification conditions indicated that the exemplary purification process that involved high pH wash conditions <pH 10.9, when combined with benzonase and MNase treatment, resulted in highly pure AAV samples (FIG. 9A). In addition, DNA agarose gel electrophoresis of the purified AAV samples indicated the presence of the ITR-transgene contained within the AAV (FIG. 9B).

These results indicated that the exemplary purification process involving a high pH wash (˜pH 9.5-pH 10.4) during the affinity chromatography purification could improve the purity of the purified AAV. Further, the results demonstrate the impurities can be measured by the exemplary assay involving gel electrophoresis and silver staining.

### Example VI: Measurement of AAV Particles with Little to No Impurities

This example establishes that AAV particle impurities can be measured using Dynamic Light Scattering (DLS), and that Differential Scanning Fluorimetry (DSF) can be used for determination of the temperature at which protein aggregation Tagg aligns with the melting of the virus.

The thermal stability as a determinant of AAV serotype identity is known in the art, and can also be used to detect impurities in AAV particle samples. For examples, as the impurities as eliminated, the onset of protein aggregation (Tagg) aligns with the melting temperature (Tm) of virus.

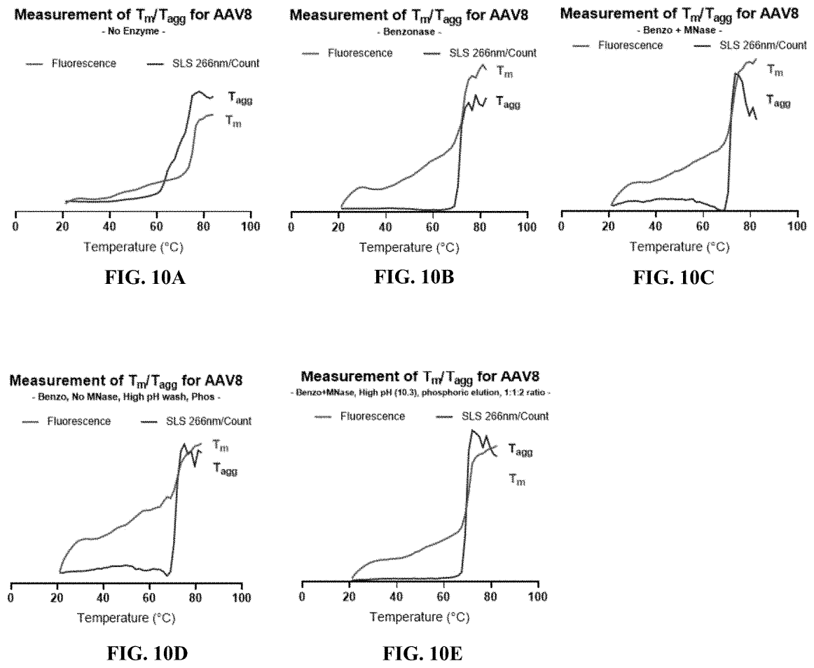

To determine whether impurities were presented in the AAV particles following purification various purification methods were performed, and the samples were measured by DLS over a range of temperatures to determine the Tm and Tagg. Consistent with previous results described above, the exemplary method involving MNase was able to improve the removal of impurities from the AAV particles. Specifically, in samples that were not treated with benzonase or MNase, the Tagg and Tm were found to differ by more than 10° C. (FIG. 10A). While the addition of benzonase improved the removal of impurities, benzonase and MNase together showed an improvement in the Tagg and Tm alignment (FIG. 10B and FIG. 10C).

Similarly, an additional exemplary purification technique involving MNase and high pH elution conditions showed that purified AAV particles could be generated that have Tagg and Tm alignment. Specifically, the Tagg and Tm were aligned in samples that were treated with benzonase and MNase and eluted with phosphoric acid (pH 10.3) (FIG. 10E) was enhanced, compared to samples that were treated with benzonase only, and eluted with phosphoric acid (pH 10.3) (FIG. 10D).

Taken together, these results demonstrated that impurities in the purified AAV particles could be substantially removed, and that DLS and DSF are exemplary assays for measuring AAV particle impurities.

### Example VII: Detection of Process-Related Impurities in Purified AAV Particles

This example establishes that AAV particles can be purified to be substantially free of process related impurities, such as host-cell DNA and host-cell proteins.

To determine whether any residual host-cell DNA was present in purified AAV particle samples following anion-exchanges (AEX) purification, an AlphaLISA assay was performed to detect host-cell DNA as the analyte. The AlphaLISA bead-based technology relies on PerkinElmer's amplified luminescent proximity homogeneous assay and uses a luminescent oxygen-channeling chemistry to detect host-cell DNA (see Beaudet et al., Nat Methods 5, an8-an9 (2008)).

Analysis of residual host-cell DNA following AEX purification was measured by AlphaLISA in samples prepared under six different conditions: (1) purification without benzonase or MNase (“no enzyme”); (2) purification with benzonase and citrate elution (“B, citrate (affinity)”); (3) purification with benzonase and citrate elution (“B, citrate”); (4) purification with benzonase, MNase, and phosphoric acid elution (“B, M, Phos”); (5) purification with benzonase, high pH (pH 10.3) wash, and phosphoric acid elution (“B, pH 10.3, Phos”); and (6) purification with benzonase, MNase, and phosphoric acid elution (“B, M, pH 10.3, Phos”). A standard curve was generated to extrapolate the concentration levels of host-cell DNA present.

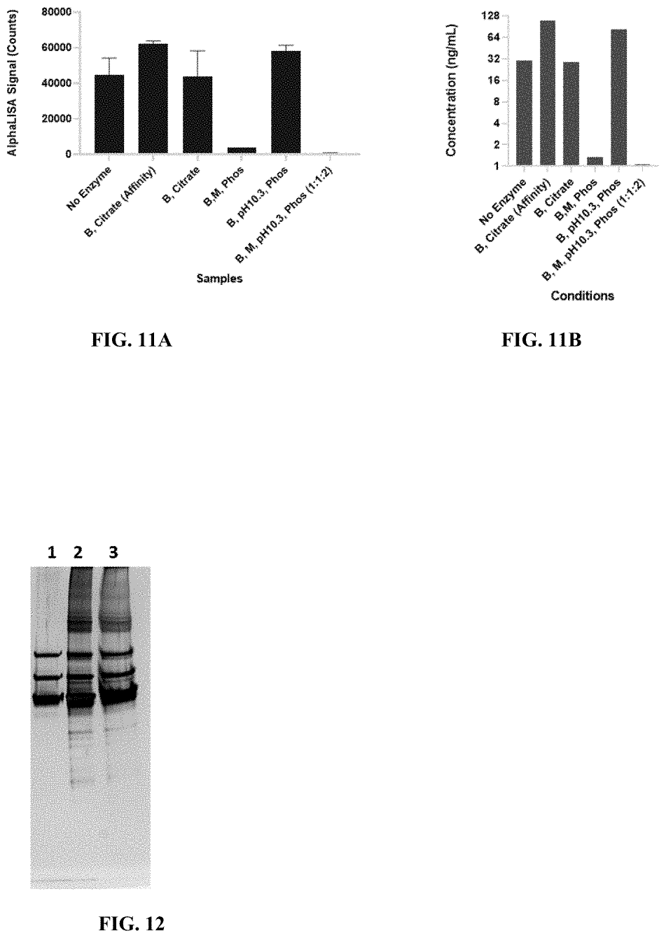

The results indicated that in the exemplary purification techniques that included MNase, AAV particles could be purified to be substantially free of host-cell DNA (FIG. 11A and FIG. 11B). For example, purification with benzonase, MNase, and phosphoric acid elution (“B, M, pH 10.3, Phos”) resulted in nearly undetectable levels of host-cell DNA (FIG. 11A and FIG. 11B).

These results demonstrated that process-related impurities, such as host-cell DNA, could be substantially removed from purified AAV particles.

### Example VIII: MNase Treatment Improves Purification Process

This example illustrates that the exemplary AAV purification method described herein involving MNase represents an improvement over the state of the art, and can generate purified AAV particles that have substantially fewer impurities.

Current methods of purification of AAV particles using anion-exchange and benzonase have been described (see, Wang C, et al. Mol Ther Methods Clin Dev. 2019; 15:257-263). However, MNase is not employed in such methods.

To determine whether the purification method described herein that includes MNase treatment can perform better than the current purification methods, the MNase treatment protocol (Protocol #1) was compared to the fractions obtained using the Wang et al. method (Protocol #2).

Briefly, purification according to Wang et al. was performed by affinity resin. Before loading to the column, the bulk AAV pool was treated with 50 U/mL benzonase at 37° C. for 1 h and clarified by centrifugation at 10,000×g for 15 min, followed by sequential filtrations through 1.2- and 0.45 mm filters. AAV was eluted from column with low-pH buffer. In parallel, a different set of samples were purified according to the methods described herein, which included MNase treatment.

Comparison between Protocol #1 and Protocol #2 revealed that MNase treatment was able to greatly reduce the amount of impurities present in the AAV particle samples, as measured by silver stain (FIG. 12).

Thus, this example demonstrates that the purification methods provided herein using MNase represent an improvement over the methods used for AAV purification.

### Example IX: Improved Stability of Purified AAVs Under Alternative Formulation Conditions

This example establishes that the formulations provided herein have improved stability through freeze/thaw stress and extended storage at −80° C.

Briefly, AAV8(GFP) was used as an exemplary AAV particle, and purified according to the methods described herein, which included MNase treatment, affinity chromatography, and anion-exchange chromatography. Following production and purification, 4.6×10¹³ VG/mL AAV particles were formulated in the exemplary buffer of Formulation #2 (20 mM Tris, 100 mM NaCl, 5% Trehalose, 0.001% Poloxamer-188, pH 7.5). Subsequently, the AAV8(GFP) particle formulation was diluted in the Formulation #2 buffer to a final concentration of 2.0×10¹³ VG/mL AAV particles. Samples were aliquoted into (a) 1.2 mL labeled cryo-vials; (b) 0.1 mL into DLS vials; and (c) 0.15 mL into SEC-FLD vials, and placed into a −80° C. freezer.

The physical properties of the AAV particles in the formulation were then characterized before and after free/thaw and extended storage at −80° C. using dynamic light scatter (DLS) and size-exclusion chromatography linked to fluorescence detection (SEC-FLD). Thawing was performed by removing the samples from the −80° C. freezer and leaving at room temperature for more than 30 minutes to thaw. Pulled SEC samples were refrozen after thawing.

A summary of the time points at which DLS and SEC-FLD were measured is providing in Table 4 and Table 5, respectively.

TABLE 4
DLS Sample Summary

| Sample | Temperature (° C.) | Time Point (Month) |
|---|---|---|
| AAV8(GFP) | Initial | 0 |
| AAV8(GFP) | −80 | 1 |
| AAV8(GFP) | −80 | 3 |
| AAV8(GFP) | −80 | 6 |
| AAV8(GFP) | −80 | F/T 1 |
| AAV8(GFP) | −80 | F/T 2 |
| AAV8(GFP) | −80 | F/T 3 |
| AAV8(GFP) | −80 | F/T 4 |
| AAV8(GFP) | −80 | FIT 5 |

TABLE 5
SEC-FLD Sample Summary

| | | Time Point |
|---|---|---|
| Sample | Temperature (° C.) | (Month) |
| AAV8(GFP) | Initial | 0 |
| AAV8(GFP) | −80 | 1 |
| AAV8(GFP) | −80 | 3 |
| AAV8(GFP) | −80 | 6 |
| AAV8(GFP) | −80 | F/T 1 |
| AAV8(GFP) | −80 | F/T 3 |
| AAV8(GFP) | −80 | F/T 5 |

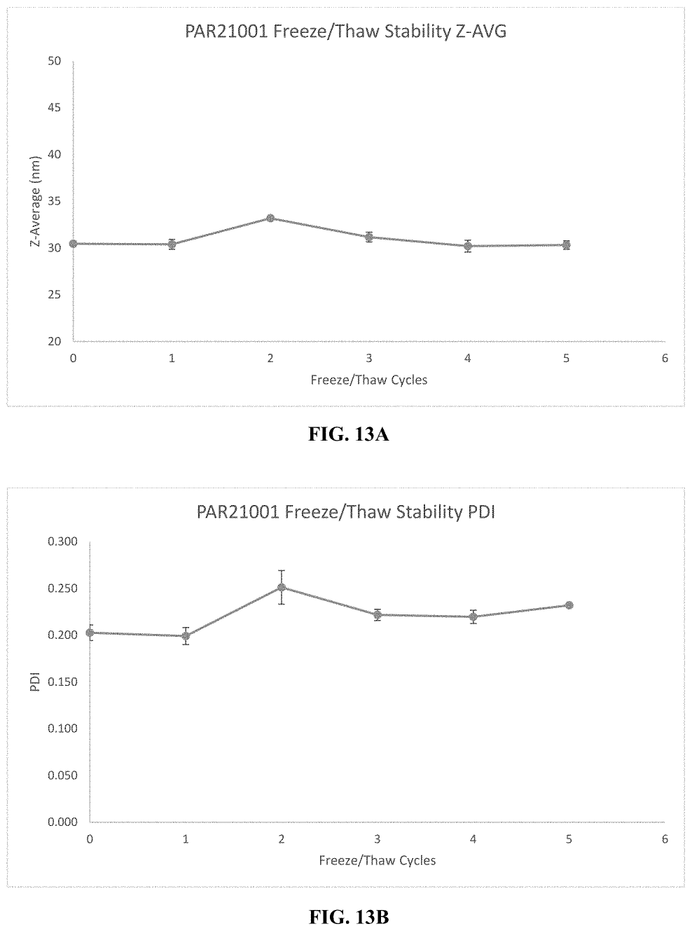

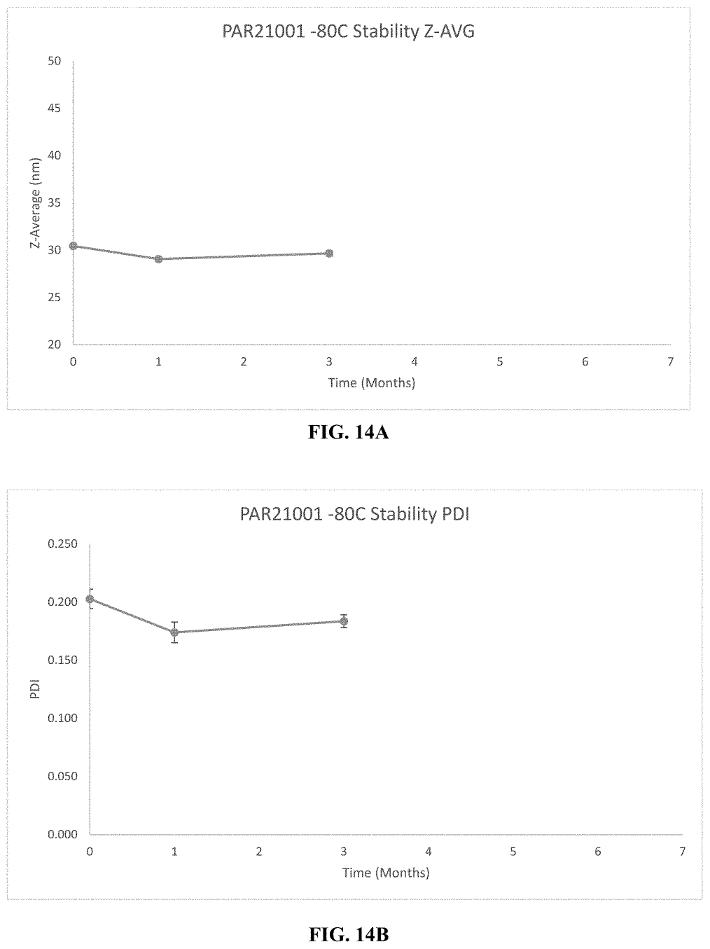

The results demonstrated robust stability of the exemplary Formulation #2 through five freeze/thaw cycles and through at least three months at −80° C. storage. For example, strong physical stability was observed, as measured by DLS particle size relative to pre-freeze DLS (t0) through freeze/thaw and −80° C. C storage, as shown in FIG. 13A-FIG. 13B and FIG. 14A-FIG. 14B, as well as Tables 6 and 7. Specifically, there was little change in particle size or polydispersity throughout.

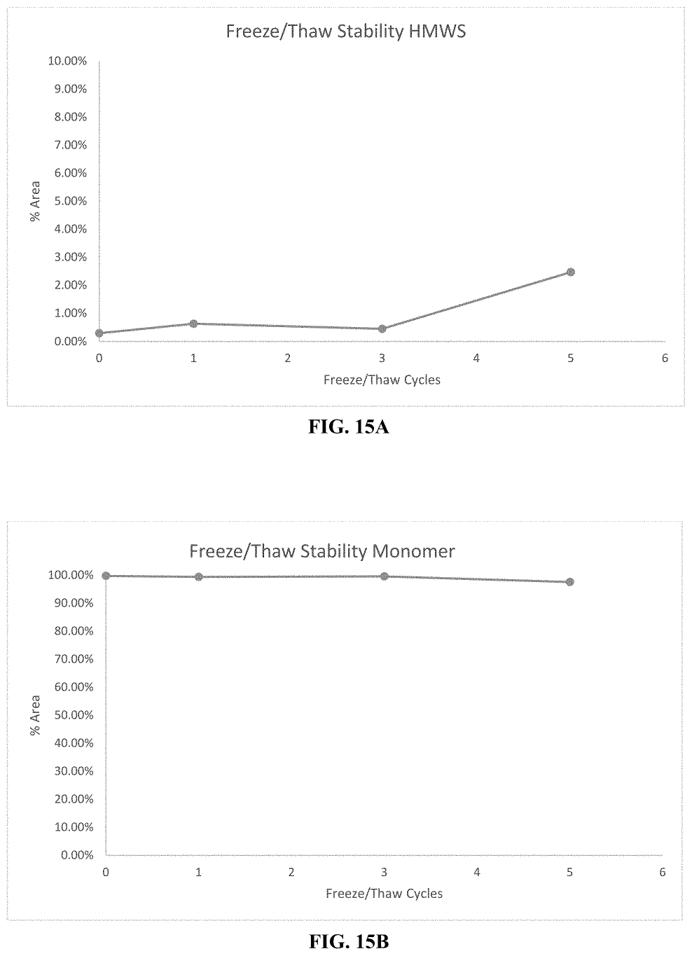

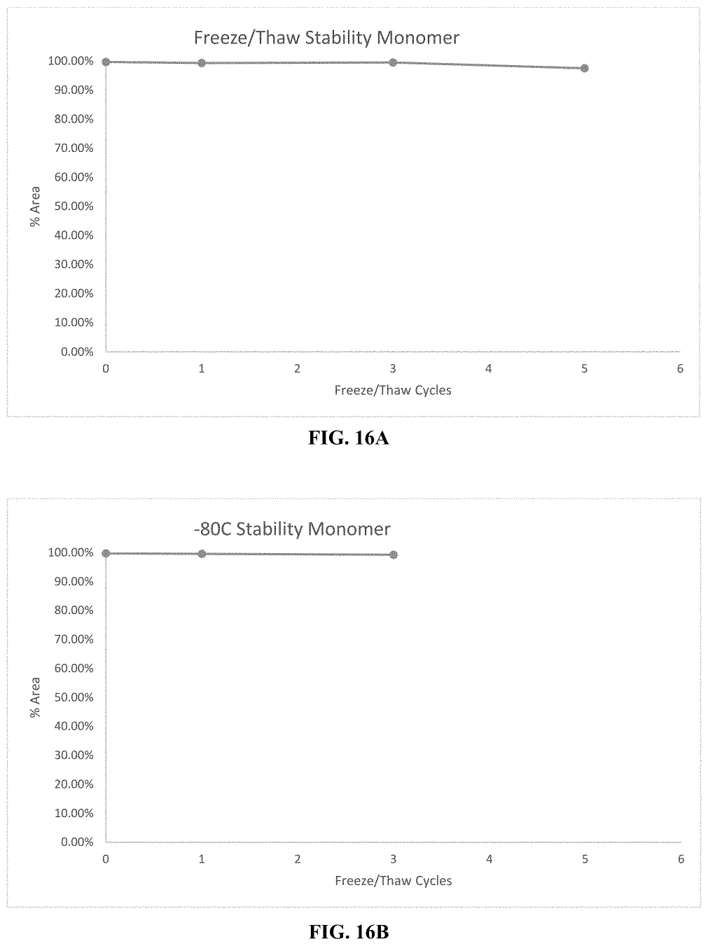

Further, as shown in FIG. 15A-FIG. 15B, and FIG. 16A-FIG. 16B, as well as Tables 8 and 9, very strong % monomer was demonstrated by SEC-FLD and very little high molecular weight species. (HMWS) were identified through freeze/thaw and −80° C. storage, which indicated very little aggregation.

TABLE 6
DLS Results for Freeze/Thaw

| Freeze/Thaws | Z-Average (nm) | Z STDEV | PDI | PDI STDEV |
|---|---|---|---|---|
| 0 | 30 | 0.30 | 0.203 | 0.008 |
| 1 | 30 | 0.53 | 0.199 | 0.009 |
| 2 | 33 | 0.18 | 0.251 | 0.018 |
| 3 | 31 | 0.52 | 0.222 | 0.006 |
| 4 | 30 | 0.63 | 0.220 | 0.007 |
| 5 | 30 | 0.45 | 0.232 | 0.002 |

PDI = Polydispersity Index;
STDEV = standard deviation.

TABLE 7
DLS Results for −80° C. Stability

| Time (Months) | Z-Average (nm) | Z STDEV | PDI | PDI STDEV |
|---|---|---|---|---|
| 0 | 30 | 0.30 | 0.203 | 0.008 |
| 1 | 29 | 0.23 | 0.174 | 0.009 |
| 3 | 30 | 0.28 | 0.184 | 0.006 |

PDI = Polydispersity Index;
STDEV = standard deviation.

TABLE 8
SEC-FLD Results for Freeze/Thaw

| Cycle | HMWS | Monomer |
|---|---|---|
| 0 | 0.29% | 99.71% |
| 1 | 0.63% | 99.37% |
| 3 | 0.45% | 99.55% |
| 5 | 2.47% | 97.53% |

HMWS = high molecular weight species.

TABLE 9
SEC-FLD Results for −80° C. Stability

| Time (Months) | HMWS | Monomer |
|---|---|---|
| 0 | 0.29% | 99.71% |
| 2 | 0.47% | 99.53% |
| 3 | 0.76% | 99.24% |

HMWS = high molecular weight species.

Taken together, these results demonstrate that the exemplary Formulation #2 has strong stability for purified AAV formulations.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

1. A pharmaceutical composition comprising a purified adeno-associated virus (AAV) particle, a buffering agent, a cryoprotectant, and a non-ionic surfactant, wherein
(a) the purified AAV particle is substantially free of an impurity,
(b) the buffering agent concentration is about 0 mM to about 50 mM,
(c) the cryoprotectant is about 1% to about 10% (w/v), and
(d) the non-ionic surfactant is about 0.0001% (w/v) to about 0.1% (w/v),
wherein optionally the pharmaceutical composition further comprises a pharmaceutically acceptable salt,
wherein optionally the pharmaceutically acceptable salt concentration is:
(i) about 1 mM to about 200 mM,
(ii) about 1 mM to about 49 mM,
(iii) about 5 mM to about 45 mM,
(iv) about 7.5 mM to about 40 mM,
(v) about 10 mM to about 30 mM,
(vi) about 10 mM to about 150 mM,
(vii) about 10 mM,
(viii) about 100 mM, or
(ix) about 150 mM,
wherein optionally the pharmaceutically acceptable salt is selected from the group consisting of a sodium salt, a magnesium salt, a calcium salt, a potassium salt, a phosphate salt, and a sulfate salt, and
wherein optionally the sodium salt comprises sodium chloride.

2-5. (canceled)

6. The pharmaceutical composition of claim 1, wherein the buffering agent comprises Tris HCl or L-Histidine HCl,
wherein optionally the buffering agent concentration is about 20 mM.

7. (canceled)

8. The pharmaceutical composition of claim 1, wherein the cryoprotectant is:
(a) about 3% (w/v) to about 8% (w/v);
(b) about 4% (w/v) to about 6% (w/v); or
(c) about 5% (w/v),
wherein optionally the cryoprotectant comprises a sugar, and
wherein optionally the sugar comprises sucrose, trehalose, or a combination thereof.

9-10. (canceled)

11. The pharmaceutical composition of claim 1, wherein the non-ionic surfactant is:
(a) about 0.0005% (w/v) to about 0.005% (w/v);
(b) about 0.00075% (w/v) to about 0.0025% (w/v); or
(c) about 0.001% (w/v),
wherein optionally the non-ionic surfactant is
(i) a copolymer,
wherein optionally the copolymer comprises a poloxamer, and
wherein optionally the poloxamer comprises poloxamer 188 (P188), poloxamer 237 (P237), poloxamer 338 (P338), poloxamer 407 (P407), or a combination thereof.
(ii) a polyoxyethylene sorbitan ester,
wherein optionally the polyoxyethylene sorbitan ester is polysorbate-20 (PS-20), polysorbate 80 (PS-80), or a combination thereof,
(iii) a phospholipid, a Brij surfactant,
(iv) a sorbitan ester, or
(v) a combination of (i)-(iv).

12. (canceled)

13. The pharmaceutical composition of claim 1, wherein:
(a) (i) the buffering agent concentration is about 20 mM,
wherein optionally the buffering agent comprises Tris hydrochloride (HCl) or L-Histidine HCl,
(ii) the cryoprotectant is about 5% (w/v) trehalose, and
(iii) the non-ionic surfactant is about 0.001% (w/v) poloxamer 188; or
(b) (i) the pharmaceutical composition comprises a pharmaceutically acceptable salt,
wherein the pharmaceutically acceptable salt is sodium chloride,
wherein optionally the concentration of the sodium chloride is about 10 mM, about 25 mM, about 50 mM, about 100 mM, about 125 mM, about 150 mM, or about 200 mM
(ii) the buffering agent concentration is about 20 mM,
wherein optionally the buffering agent comprises Tris hydrochloride (HCl) or L-Histidine HCl,
(iii) the cryoprotectant is about 5% (w/v) trehalose, and
(iv) the non-ionic surfactant is about 0.001% (w/v) poloxamer 188.

14-15. (canceled)

16. The pharmaceutical composition of claim 1, wherein
(A) the pharmaceutical composition pH is:
(a) about 4.0 to about 9.0;
(b) about 7.0 to about 8.0;
(c) about 7.3 to about 7.7; or
(d) about 7.5;
(B) the AAV
(a) is selected from the group consisting of AAV1, AAV2, AAV3, AAV4, AAV5, AAV6, AAV7, AAV8, AAV9, and AAV10, and/or
(b) the AAV comprises a recombinant AAV (rAAV);
(C) the purified AAV particle titer is:
(a) about 1×10¹⁰ viral genomes per milliliter (vg/mL) or greater;
(b) about 1×10¹¹ viral genomes per milliliter (vg/mL) or greater;
(c) about 1×10¹² viral genomes per milliliter (vg/mL) or greater;
(d) about 1×10¹³ viral genomes per milliliter (vg/mL) or greater;
(e) about 1×10¹⁴ viral genomes per milliliter (vg/mL) or greater; or
(f) about 1×10¹⁵ viral genomes per milliliter (vg/mL) or greater; and/or
(D) the impurity comprises
(a) a process-related impurity,
wherein the process-related impurity is optionally selected from the group consisting of a residual host-cell component, a residual viral production component, a residual cell culture component, a residual purification component, or a combination thereof,
wherein the residual host-cell component optionally comprises a host-cell protein, a host-cell DNA, a host-cell RNA, or a combination thereof,
wherein the host-cell DNA optionally comprises an extra-viral, chromatin-associated DNA,
wherein the residual viral production component optionally comprises a plasmid DNA, a helper virus, or a combination thereof,
wherein the residual cell culture component optionally comprises an antibiotic, a supplement, an inducer, a growth factor, or a combination thereof, and/or
wherein the residual purification component optionally comprises a buffer, an inorganic salt, an enzyme, a detergent, a medium, or a combination thereof:
(b) a product-related impurity, wherein the product-related impurity comprises an empty capsid, an aggregated AAV particle, a degraded AAV particle, or a combination thereof, or
(c) a combination of (a) and (b),
wherein optionally the purified AAV particle:
(i) comprises a full or a partially-full capsid, and the product-related impurity comprises an empty capsid,
(ii) comprises a full capsid, and the product-related impurity comprises an empty capsid, or
(iii) essentially of a full capsid, and the product-related impurity comprises an empty capsid.

17-23. (canceled)

24. The pharmaceutical composition of claim 1, wherein the pharmaceutical composition is in:
(a) a liquid state; or
(b) a solid or a semi-solid state.

25. The pharmaceutical composition of claim 1, wherein the pharmaceutical composition
(a) maintains or enhances the stability of the purified AAV particle; and/or
(b) reduces or prevents aggregation of the purified AAV particle,
optionally, wherein
(a) the stability of the purified AAV particle is maintained or enhanced after
(i) one or more freeze/thaw cycles; or
(ii) after three or more freeze/thaw cycles; and/or
(b) the aggregation of the AAV particle is
(i) less than 5% after one or more freeze/thaw cycles;
(ii) less than 2% after one or more freeze/thaw cycles;
(iii) less than 1% after one or more freeze/thaw cycles;
(iv) less than 5% after three or more freeze/thaw cycles;
(v) less than 2% after three or more freeze/thaw cycles; or
(vi) less than 1% after three or more freeze/thaw cycles, and
wherein the stability and/or aggregation of the AAV particle is optionally measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

26. (canceled)

27. The pharmaceutical composition of claim 1, wherein the purified AAV particle is obtained by a method comprising:
(a) contacting a supernatant comprising AAV particles with a composition comprising a nuclease; and
(b) purifying the particles,
wherein the nuclease optionally comprises Benzonase, or Benzonase® and a chromatin-DNA nuclease, and
wherein the chromatin-DNA nuclease optionally comprises a MNase.

28. A method for making a pharmaceutical composition comprising a purified AAV particle, the method comprising:
(A) contacting a supernatant comprising an AAV particle with a composition comprising a nuclease,
wherein the nuclease optionally comprises Benzonase®, or Benzonase® and a chromatin-DNA nuclease, and
wherein the chromatin-DNA nuclease optionally comprises a MNase;
(B) purifying the AAV particle, such that the AAV particle is substantially free of an impurity;
(C) combining the purified AAV particle with a buffering agent, a cryoprotectant, and a non-ionic surfactant, wherein
(a) the buffering agent concentration is about 0 mM to about 50 mM,
(b) the cryoprotectant is about 1% to about 10% (w/v), and
(c) the non-ionic surfactant is about 0.0001% (w/v) to about 0.1% (w/v),
optionally wherein step (C) further comprises combining a pharmaceutically acceptable salt, wherein the pharmaceutically acceptable salt concentration is:
(i) about 1 mM to about 200 mM,
(ii) about 10 mM to about 150 mM,
(iii) about 1 mM to about 49 mM,
(iv) about 5 mM to about 45 mM,
(v) about 7.5 mM to about 40 mM,
(vi) about 10 mM to about 30 mM,
(vii) about 10 mM,
(viii) about 100 mM, or
(ix) about 150 mM,
wherein optionally the pharmaceutically acceptable salt comprises a sodium salt, a magnesium salt, a calcium salt, a potassium salt, a phosphate salt, or a sulfate salt.

29-30. (canceled)

31. The method of claim 28, wherein the buffering agent comprises Tris HCl or L-Histidine HCl;
wherein optionally, the buffering agent is at a concentration of about 20 mM.

32. (canceled)

33. The method of claim 28, wherein the cryoprotectant is:
(a) about 3% (w/v) to about 8% (w/v);
(b) about 4% (w/v) to about 6% (w/v); or
(c) about 5% (w/v),
wherein optionally the cryoprotectant comprises a sugar,
wherein optionally the sugar comprises sucrose, trehalose, or a combination thereof.

34-35. (canceled)

36. The method of claim 28, wherein the non-ionic surfactant is
(a) about 0.0005% (w/v) to about 0.005% (w/v);
(b) about 0.00075% (w/v) to about 0.0025% (w/v); or
(c) about 0.001% (w/v),
wherein optionally the non-ionic surfactant is
(i) a copolymer,
wherein optionally the copolymer comprises a poloxamer, and
wherein optionally the poloxamer comprises poloxamer 188 (P188), poloxamer 237 (P237), poloxamer 338 (P338), poloxamer 407 (P407), or a combination thereof.
(ii) a polyoxyethylene sorbitan ester,
wherein optionally the polyoxyethylene sorbitan ester is polysorbate-20 (PS-20), polysorbate 80 (PS-80), or a combination thereof,
(iii) a phospholipid, a Brij surfactant,
(iv) a sorbitan ester, or
(v) a combination of (i)-(iv).

37. (canceled)

38. The method of claim 28, wherein the pharmaceutical composition pH is
(a) about 4.0 to about 9.0;
(b) about 7.0 to about 8.0;
(c) about 7.3 to about 7.7; or
(d) about 7.5.

39. The method of claim 28, wherein the AAV is selected from the group consisting of AAV1, AAV2, AAV3, AAV4, AAV5, AAV6, AAV7, AAV8, AAV9, and AAV10; and/or
wherein the AAV comprises a rAAV.

40. The method of claim 28, wherein the purified AAV particle titer is:
(a) about 1×10¹⁰ viral genomes per milliliter (vg/mL) or greater;
(b) about 1×10¹¹ viral genomes per milliliter (vg/mL) or greater;
(c) about 1×10¹² viral genomes per milliliter (vg/mL) or greater;
(d) about 1×10¹³ viral genomes per milliliter (vg/mL) or greater;
(e) about 1×10¹⁴ viral genomes per milliliter (vg/mL) or greater; or
about 1×10¹⁵ viral genomes per milliliter (vg/mL) or greater.

41. The method of claim 28, wherein the impurity comprises
(a) a process-related impurity,
wherein the process-related impurity is optionally selected from the group consisting of a residual host-cell component, a residual viral production component, a residual cell culture component, a residual purification component, or a combination thereof,
wherein the residual host-cell component optionally comprises a host-cell protein, a host-cell DNA, a host-cell RNA, or a combination thereof,
wherein the host-cell DNA optionally comprises an extra-viral, chromatin-associated DNA,
wherein the residual viral production component optionally comprises a plasmid DNA, a helper virus, or a combination thereof,
wherein the residual cell culture component optionally comprises an antibiotic, a supplement, an inducer, a growth factor, or a combination thereof, and/or
wherein the residual purification component optionally comprises a buffer, an inorganic salt, an enzyme, a detergent, a medium, or a combination thereof;
(b) a product-related impurity, wherein the product-related impurity comprises an empty capsid, an aggregated AAV particle, a degraded AAV particle, or a combination thereof; or
(c) a combination of (a) and (b),
wherein optionally the purified AAV particle:
(a) comprises:
(i) a full or a partially-full capsid, and the product-related impurity comprises an empty capsid; or
(ii) a full capsid, and the product-related impurity comprises an empty capsid; or
(b) consists essentially of a full capsid, and the product-related impurity comprises an empty capsid;
wherein optionally the product-related impurity comprises
(a) an aggregated AAV particle, a degraded AAV particle, or a combination thereof; or
(b) an aggregated AAV particle;
wherein optionally the purified AAV particle comprises or consists essentially of an empty capsid.

42-45. (canceled)

46. The pharmaceutical composition of claim 13, wherein
(A) the pharmaceutical composition pH is:
(a) about 4.0 to about 9.0;
(b) about 7.0 to about 8.0;
(c) about 7.3 to about 7.7; or
(d) about 7.5;
(B) the AAV
(a) is selected from the group consisting of AAV1, AAV2, AAV3, AAV4, AAV5, AAV6, AAV7, AAV8, AAV9, and AAV10, and/or
(b) the AAV comprises a recombinant AAV (rAAV);
(C) the purified AAV particle titer is:
(a) about 1×10¹⁰ viral genomes per milliliter (vg/mL) or greater;
(b) about 1×10¹¹ viral genomes per milliliter (vg/mL) or greater;
(c) about 1×10¹² viral genomes per milliliter (vg/mL) or greater;
(d) about 1×10¹³ viral genomes per milliliter (vg/mL) or greater;
(e) about 1×10¹⁴ viral genomes per milliliter (vg/mL) or greater; or
(f) about 1×10¹⁵ viral genomes per milliliter (vg/mL) or greater; and/or
(D) the impurity comprises
(a) a process-related impurity,
wherein the process-related impurity is optionally selected from the group consisting of a residual host-cell component, a residual viral production component, a residual cell culture component, a residual purification component, or a combination thereof,
wherein the residual host-cell component optionally comprises a host-cell protein, a host-cell DNA, a host-cell RNA, or a combination thereof,
wherein the host-cell DNA optionally comprises an extra-viral, chromatin-associated DNA,
wherein the residual viral production component optionally comprises a plasmid DNA, a helper virus, or a combination thereof,
wherein the residual cell culture component optionally comprises an antibiotic, a supplement, an inducer, a growth factor, or a combination thereof, and/or
wherein the residual purification component optionally comprises a buffer, an inorganic salt, an enzyme, a detergent, a medium, or a combination thereof;
(b) a product-related impurity, wherein the product-related impurity comprises an empty capsid, an aggregated AAV particle, a degraded AAV particle, or a combination thereof; or
(c) a combination of (a) and (b),
wherein optionally the purified AAV particle:
(i) comprises a full or a partially-full capsid, and the product-related impurity comprises an empty capsid;
(ii) comprises a full capsid, and the product-related impurity comprises an empty capsid; or
(iii) essentially of a full capsid, and the product-related impurity comprises an empty capsid.

47. The pharmaceutical composition of claim 13, wherein the pharmaceutical composition is in:
(a) a liquid state; or
(b) a solid or a semi-solid state.

48. The pharmaceutical composition of claim 13, wherein the pharmaceutical composition
(a) maintains or enhances the stability of the purified AAV particle; and/or
(b) reduces or prevents aggregation of the purified AAV particle,
wherein optionally
(a) the stability of the purified AAV particle is maintained or enhanced after
(i) one or more freeze/thaw cycles; or
(ii) after three or more freeze/thaw cycles; and/or
(b) the aggregation of the AAV particle is
(i) less than 5% after one or more freeze/thaw cycles;
(ii) less than 2% after one or more freeze/thaw cycles;
(iii) less than 1% after one or more freeze/thaw cycles;
(iv) less than 5% after three or more freeze/thaw cycles;
(v) less than 2% after three or more freeze/thaw cycles; or
(vi) less than 1% after three or more freeze/thaw cycles, and
wherein the stability and/or aggregation of the AAV particle is optionally measured by an assay selected from the group consisting of dynamic light scattering (DLS), analytical ultracentrifugation (AUC), light microscopy, size exclusion chromatography (SEC), transmission electron microscopy, and field flow fractionation with multi-angle static light scattering (FFF-MALS).

49. The pharmaceutical composition of claim 1, wherein the purified AAV particle is obtained by a method comprising:
(a) contacting a supernatant comprising AAV particles with a composition comprising a nuclease; and
(b) purifying the particles,
wherein the nuclease optionally comprises Benzonase, or Benzonase® and a chromatin-DNA nuclease, and
wherein the chromatin-DNA nuclease optionally comprises a MNase.
